(19) United States
(12) Patent Application Publication
(10) Pub. No.: US 2023/0187019 A1
(43) Pub. Date: Jun. 15, 2023

(54) METHOD FOR DETERMINING A DRUG COMBINATION VIA A BLOCKING MODEL OF A MULTI-SITE-TARGETED PROTEIN AND APPLICATIONS THEREOF

(71) Applicant: National Tsing Hua University, Hsinchu City (TW)

(72) Inventors: Lee-Wei YANG, Garland, TX (US); Kun-Lin TSAI, Hsinchu City (TW)

(21) Appl. No.: 18/066,103

(22) Filed: Dec. 14, 2022

Related U.S. Application Data
(60) Provisional application No. 63/289,176, filed on Dec. 14, 2021.

(51) Int. Cl. G16B 15/30; A61K 31/4178; A61K 31/4745; A61P 35/00; A61K 31/5025; A61K 31/365

(52) U.S. Cl. CPC G16B 15/30; A61K 31/4178; A61K 31/4745; A61P 35/00; A61K 31/5025; A61K 31/365

(57) ABSTRACT

Provided is a method for determining a drug combination targeting different sites of a protein or a protein complex, including: identifying a first binding site and a second binding site of the protein or the protein complex based on a three-dimensional structure thereof, wherein the first binding site and the second binding site are different sites in the three-dimensional structure of the protein or the protein complex; identifying a first drug interacting with the first binding site; identifying a second drug interacting with the second binding site; and combining the first drug and the second drug to provide at least one of a synergistic effect and an additive effect in suppressing an activity of the protein or the protein complex. Also provided is a method for treating a ATG4B-related disease or a 3C-like protease-related disease by the drug combination.

### TECHNICAL FIELD

The present disclosure relates generally to methods of determining a drug combination targeting a protein or a protein complex, and more particularly, to methods for suppressing the activity of a protein or a protein complex by the drug combination via constituent drugs targeting multiple sites in a protein or a protein complex (multi-site-targeting strategy).

### Sequence Listing

The present application is being filed along with a Sequence Listing in electronic format. The Sequence Listing is provided as a file entitled 210761US-Sequence Listing-yym-wll-chl-bjp-20221227.xml, created on Dec. 23, 2022, which is 20.5 kb in size. The information in the electronic format of Sequence Listing is incorporated herein by reference in its entirety.

### BACKGROUND

Autophagy guards the homeostasis of cells to adapt to various internal and external stresses, such as starvation, oxidation stress, aging organelles, dysfunctional proteins, and invaded pathogens. Abnormally regulated autophagy has been found to be associated with both the initiation and progression of cancers (Singh et al., 2017, Oncogene 37, 1142-1158). Recently, one of the autophagins, autophagy related 4B cysteine peptidase (ATG4B), has received more and more attention due to its potential as a drug target for tumor suppression in view of the increasing evidence of its upregulated expression level in several tumor tissues (Liu et al., 2014, Autophagy 10, 1454-1465; Bortnik et al., 2016, Oncotarget 7, 66970-66988; Rothe et al., 2014, Blood 123, 3622-3634; Han et al., 2010, Oncology Letters 1, 821-826; Peng et al., 2017, Oncogenesis 6, e292) and the slight side effects in ATG4B-defected mice (Read et al., 2010, Veterinary Pathology 48, 486-494).

ATG4B is also known to be one of the proteins involved in formation of autophagosome, a mature vehicle that can fuse with the lysosome for degradation of its enclosed substances (Yang et al., 2010, Nature Cell Biology 12, 814-822). The autophagosome originates from the phagophore, an isolated membrane fragment secreted from surrounding organelles, such as endoplasmic reticulum (ER) and mitochondria (Lamb et al., 2013, Nature Reviews Molecular Cell Biology 14, 759-774). The extension of the phagophore requires the phosphatidylethanolamine (PE)-bound microtubule-associated-protein 1 light chain 3B (LC3B-II) on the membrane, while the maturation of autophagosome demands the cleavage of LC3B-II to release free-form LC3B (LC3B-I) (Bento et al., 2016, Annual Review of Biochemistry 85, 685-713). ATG4B participates in the formation of autophagosome by, as a cysteine protease, cleaving the last four residues of the full-length LC3B (pro-LC3B, 1-124) to generate LC3B-I (1-120), leaving a C-terminus glycine that is then activated by the El-like enzyme ATG7 (autophagy related 7) and covalently bonded with phosphatidylethanolamine (PE) (LC3B-II) mediated by E2-like enzyme ATG3 (autophagy related 3) and the ATG12-ATG5-ATG16 conjugation system (Maruyama et al., 2017, The Journal of Antibiotics 71, 72-78; Bento et al., 2016, Annual Review of Biochemistry 85, 685-713).

On the other hand, ATG4B is also responsible for the cleavage of LC3B-II for the mature autophagosome and regeneration of LC3B-I for the next cycle of the autophagosome formation (Yu et al., 2012, Autophagy 8, 883-892). It is also suggested that the LC3B-II cleavage capability of ATG4B also serves as a correction mechanism to reverse the mis-conjugation of LC3B on other organelle membranes and maintain the abundance of LC3B-I for autophagosome formation (Nakatogawa et al., 2012, Autophagy 8, 177-186). The ATG4B depletion cells result in accumulated LC3B puncta, which indicates the abnormal accumulation of autophagosome (Liu et al., 2018, Theranostics 8, 830-845; Bortnik et al., 2016, Oncotarget 7, 66970-66988).

A couple of ATG4B inhibitors identified by in silico drug screening have been reported. Akin et al. performed structure-based virtual screening on closed-form ATG4B to dock 139,735 compounds from the National Cancer Institute (NCI) database and identified compound, NSC185058, which docked at the exit of the active site nearby the ATG4B N-terminus with IC₅₀ of 51 μM (Akin et al., 2014, Autophagy 10, 2021-2035). Bosc et al. used both an opened form and closed form of the ATG4B structure to dock 230,000 compounds from the NCI database and 500,000 compounds from ChemBridge database. They identify a hit compound that also docked at a similar pocket on the closed-form ATG4B and was optimized as a resulting compound, LV-320, with IC₅₀ of 24.5 μM and K_{d} of 16 μM (Bosc et al., 2018, Scientific Reports 8, 11653). Another compound, 5130, computationally screened from a customized library of 7,249 non-commercial compounds and predicted to bind the similar groove formed by the folded N-terminal tail was reported to specifically target on ATG4B with IC₅₀ of 3.24 μM (Fu et al., 2018, Autophagy 15, 295-311). Liu et al. combined molecular docking and molecular dynamics (MD) simulations to screen 1,312 FDA-approved drugs on the open-form ATG4B structure and repurposed an antifungal drug, tioconazole, as an ATG4B inhibitor by blocking the active site entry for LC3 C-terminus with IC₅₀ of 1.79 μM (Liu et al., 2018, Theranostics 8, 830-845).

Designing drugs specifically targeting at the protein-protein interface (PPI) is a drug development strategy that is receiving high attention. At present, there have been successful cases of drug development in the field of immunology. By targeting the PPI of tumor necrosis factor-α (TNF-α) and its receptor, three FDA-approved drugs have been found to inhibit their biochemical reactions (Palladino et al., 2003, Nature Reviews Drug Discovery 2, 736-746; Eng, G. P., 2016, “Optimizing biological treatment in rheumatoid arthritis with the aid of therapeutic drug monitoring.” Dan. Med. J. 63(11)). In fact, the design of PPI blockers requires some prerequisites; for example, the buried surface area (BSA) of the PPI does not exceed 4,000 Å² (Ran et al., 2018, Curr. Opin. Chem. Biol. 44, 75-86). Generally, the PPI of a protein that is too large or that has a smooth interface is easily regarded as “undruggable” (Ran et al., 2018, Curr. Opin. Chem. Biol. 44, 75-86). In the past, researchers used computer docking technology analysis to dock a large number of different small molecules as probes on the surface of a protein, and then classified the position of each docking result to find the “druggable” hot spots belonging to this protein for future medications.

However, it is still in the progress of finding effective drugs and formulations applicable to clinical treatment, e.g., drugs targeting different sites of a protein target, such as ATG4B and 3CL^{pro}, to achieve a better therapeutic effect on the diseases associated with the protein target.

### SUMMARY

While several ATG4B inhibitors have been reported, their potency was generally moderate, with the IC₅₀ values ranging from 80 nM to 51 μM. However, there are at least two alternative sites on ATG4B that could be druggable from the observation of the crystallized ATG4B-LC3B complex (FIGS. 1A and 1B). While the correct position of LC3B (substrate LC3B, S-LC3B) C-terminus into the active site of ATG4B is required for its cleavage, the association between ATG4B and LC3B at the recognition interface, e.g., the flat area outside and below the catalytic cavity (FIG. 1A), can be used for the enzyme-substrate recognition at the first place. On the other hand, the interaction between the ATG4B N-terminus and the LC3B from one of the adjacent lattices, i.e., the N-terminus-binding LC3B (N-LC3B), mediates the intermolecular allosteric regulation of the active site conformation from a closed state (ATG4B(C)) (FIG. 1B) to an open state (ATG4B(O)) (FIG. 1A).

Drug combination is a strategy that combines two or more drugs functioned by targeting different drug targets or pathways in one formulation to achieve an enhanced therapeutic effect while lowering down the dose usage of each individual drug. This concept might also apply to drugs targeting different sites on the same target protein for an enhanced inhibition on its biological function.

The present disclosure provides a multi-site-targeting strategy which presents a concept aiming to utilize existing pharmaceutical sources or newly synthesized compounds by designing formulations to target at least two potential druggable sites in a single protein or protein complex target. If the combination of multiple compounds can achieve a better inhibitory effect on a protein target than that of individual binders alone, the dosage and thus the toxicity of each compound could be reduced while achieving a similar or better therapeutic effect than that from individual compound alone. The multi-site-targeting strategy can ideally apply to any protein target, given the druggable binding sites available. This can promote the exhaustive use of the available medicinal space to known or new protein targets for human diseases and other emerging infectious diseases.

The druggable binding sites located in the three-dimensional structure of a protein target can be identified by both experimental techniques, such as X-ray crystallography and the chemical shift perturbation data based on nuclear magnetic resonance (NMR) spectroscopy, and computational methods, such as protein-protein docking and molecular dynamics (MD) simulations. The identification of allosteric sites could require mutation screening for protein function. In addition, computational methods, such as time-dependent and independent linear response theory (LRT), model the atomic displacement inside correlated atomic motion upon remote perturbation, and thus can probe frequently communicating residues, e.g., the possible allosteric sites. Furthermore, MD simulation and elastic network models could explore the conformational space of a target protein for docking to screen and discover effective drugs that would not otherwise be found using a single protein conformation. With the alternative druggable sites, the present disclosure provides a general framework for designing a multi-site-targeting strategy to suppress drug targets in general.

In one aspect, the present disclosure relates to a method for determining a drug combination targeting different sites of a protein or a protein complex, comprising: identifying a first binding site and a second binding site of the protein or the protein complex based on a three-dimensional structure thereof, wherein the first binding site and the second binding site are different sites in the three-dimensional structure of the protein or the protein complex; identifying a first drug interacting with the first binding site; identifying a second drug interacting with the second binding site; and combining the first drug and the second drug to provide at least one of a synergistic effect and an additive effect in suppressing an activity of the protein or the protein complex. In some embodiments, the drug combination including the first drug and the second drug provides a better effect than that of the first drug or the second drug alone.

In exemplary embodiments of the method of the present disclosure, the first binding site and the second binding site are independently a main functional site, an orthosteric site, an active site, a main substrate-binding site, an allosteric site, a recognition site or a site at the protein-protein interface.

In exemplary embodiments of the method of the present disclosure, the first drug and the second drug are independently selected from the group consisting of a newly synthesized compound, an FDA-approved drug, an FDA-approved biologic, a drug metabolite, a prodrug, an experimental small molecule, an experimental biologic, an experimental polypeptide, and any combination thereof. In some embodiments, at least one of the first drug and the second drug is an anticancer drug. In some embodiments, at least one of the first drug and the second drug is an antiviral drug (e.g., nelfinavir and boceprevir). In some embodiments, at least one of the first drug and the second drug is a non-anticancer drug such as an anthelmintic drug (e.g., moxidectin), an antibacterial drug (e.g., norvancomycin) or an antifungal drug (e.g., tioconazole).

In exemplary embodiments of the method of the present disclosure, the three-dimensional structure is an ensemble structure of the protein or the protein complex.

In exemplary embodiments of the method of the present disclosure, the structure of the protein or the protein complex can be found by a crystal structure, an NMR-determined structure, a cryo-electron microscopy (EM)-resolved structure, a simulation-sampled structure, structures predicted from a structural prediction algorithm, or a combination thereof.

In exemplary embodiments of the method of the present disclosure, the identifying of the first drug comprises selecting the first drug from at least one first dataset containing molecular entities. In some embodiments, the identifying of the second drug comprises selecting the second drug from at least one second dataset. In some embodiments, the first dataset and the second dataset are the same or different. In some embodiments, the first dataset and the second dataset are independently a drug library, a genomic dataset, a proteomic dataset, a biochemical dataset or a population dataset.

In exemplary embodiments of the method of the present disclosure, the selecting comprises interacting the molecular entities of the dataset with at least one of the first binding site and the second binding site, and ranking the affinity of the molecular entities (i.e., compounds, drugs, etc.) to at least one of the first binding site and the second binding site by experimental and/or theoretical methods, for example, nuclear magnetic resonance (NMR) spectroscopy, isothermal titration calorimetry, docking energy, distances between poses and the binding site, entropy calculations, molecular dynamics (MD) simulations, normal mode analysis (NMA), or any combination thereof.

In exemplary embodiments of the method of the present disclosure, an allosteric site can be determined by analysis of atomic displacement and/or correlated atomic motion derived from the molecular dynamics (MD) simulations, normal mode analysis (NMA), linear response theory (LRT), or any combination thereof.

In exemplary embodiments of the method of the present disclosure, poses are reported for the molecular entities docked by AutoDock Vina and/or AutoDock.

In exemplary embodiments of the method of the present disclosure, the ranking is performed by normalized ranking, logarithm of odds (LOD) scoring or a combination thereof.

In exemplary embodiments of the method of the present disclosure, the normalized ranking is performed based on at least one of docking affinity, number of contacts, and an extent of poses concentrated in at least one of the first binding site and the second binding site.

In exemplary embodiments of the method of the present disclosure, the logarithm of odds scoring is performed based on at least one of docking affinity, a distance of the molecular entity to the first binding site or the second binding site, and a size of poses cluster.

In one aspect, the present disclosure relates to a method for treating an ATG4B-related disease or a 3CL protease (3CL^{pro})-related disease in a subject in need thereof, comprising administering an effective amount of the drug combination obtained from the aforementioned methods.

In exemplary embodiments of the method of the present disclosure, the drug combination comprises at least two selected from the group consisting of aclacinomycin A, boceprevir, daclatasvir, dihydroergocristine, ethynyl estradiol, Evans blue, moxidectin, netupitant, norvancomycin, ponatinib, temsirolimus, tioconazole, tat-N7 peptide, and tat-N9 peptide.

In exemplary embodiments of the method of the present disclosure, the ATG4B-related disease is breast cancer, colorectal cancer, neural glioma cancer, gastric cancer, pancreatic cancer or melanoma.

In this disclosure, where a document, act or item of knowledge is referred to or discussed, this reference or discussion is not an admission that the document, act or item of knowledge or any combination thereof was at the priority date, publicly available, known to the public, part of common general knowledge, otherwise constitutes prior art under the applicable statutory provisions, or is known to be relevant to an attempt to solve any problem with which this specification is concerned.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a fuller understanding of this disclosure, reference should be made to the following detailed descriptions, taken in connection with the accompanying drawings.

FIGS. 1A and 1B illustrate structures of different ATG4B forms. FIG. 1A shows the structures of the open/active form of ATG4B (PDB code: 2Z0D, ATG4B(O)), and FIG. 1B shows the structures of the closed/inactive form of ATG4B (PDB code: 2CY7, ATG4B(C)), in which ATG4B is shown in white. Together with the open form, LC3 with a cleaved C-terminus docked into the active site (red protein) and LC3 from one of the adjacent crystallographic unit cells (green protein) are shown. The active site of ATG4B comprising the residues Cys74 (the catalytic cysteine), Asp278 and His280 are shown in licorice. The adjacent residues, Trp142, Pro260 and Asn261 (in licorice), form a substrate-binding cavity, with the latter two residues located at a regulatory loop that spatially flanks the cavity. The differences in the N-terminus positions in the open and closed forms of ATG4B were noted.

FIGS. 2A and 2B illustrate the ATG4B-LC3B recognition interface. FIG. 2A shows the complex structure of ATG4B and LC3B (PDB code: 2Z0D). ATG4B was colored in grey, and LC3B was colored in green. The heavy atoms composing the recognition interface were presented in sticks and colored by cyan and yellow for the interface on ATG4B and LC3B, respectively. FIG. 2B is the same as FIG. 2A but showing in isolated manner.

FIGS. 3A and 3B illustrate the distribution of the size of the largest pose clusters, which are chosen as the representative clusters and sampled from each of the 2016 FDA-approved drugs when docked on ATG4B (FIG. 3A) and LC3B (FIG. 3B). Those drugs with their representative pose clusters larger than the average size were colored in red, which are preferentially selected for the next round of drug screening procedure.

FIG. 4A is the flowchart of the virtual drug screening for ATG4B and LC3 interface. Among the retained 42 drugs, vinorelbine (Vb) could inhibit the catalysis of ATG4B for LC3B, suggested from the in vitro cleavage assay.

FIG. 4B illustrates in vitro cleavage assay for ATG4B catalytic activity. The ATG4B substrate, LC3B, was cloned and expressed with C-Myc and S-tag attached on the N and C terminus, respectively (right panel). The cleavage of the LC3B C-terminus by ATG4B (red line in the right panel) consumed the full length tagged LC3B and resulted in a lighter band on the gel (left panel). Drugs that could inhibit the catalysis of ATG4B for LC3B will resulted in intact full length LC3B, remaining a darker band on the gel. S-tag: full length LC3B tagged by S-tag. N: negative control, without ATG4B and thus all the LC3B retained full length. P: positive control, the full length LC3B was cleaved in the presence of ATG4B. Vb: vinorelbine, a drug repurposed as ATG4B-LC3B recognition interface blocker showing 58% inhibition of ATG4B catalysis.

FIGS. 5A to 5D illustrate the snapshots of the different ratio of remaining contacts (100% (FIG. 5A), 40% (FIG. 5B), 30% (FIG. 5C) and 20% (FIG. 5D)) formed between the drug and protein during the 10 ns MD simulation when compared to the pose sampled by docking. The snapshots were taken from the simulation of azimilide binding on the recognition interface on ATG4B.

FIG. 6A is the diagram of FPLC chromatogram of the GST column for GST-LC3B purification.

FIG. 6B shows the photo of SDS-PAGE of purified GST-LC3B by FPLC.

FIG. 6C is the diagram of FPLC chromatogram of the GST column for the separation of cleaved GST and LC3B.

FIG. 6D shows the photo of SDS-PAGE verifying the cleavage and separation of GST and LC3B.

FIG. 6E is the diagram of HPLC chromatogram and SDS-PAGE of the purified LC3B.

FIG. 6F shows the purified LC3B confirmed by ESI-MS. The measured ESI Mass of LC3B was close to the calculated value based on its amino acid sequence.

FIG. 7 shows the clustered ensemble structures of LC3B projected on the first two principle components (PCs). Group 1 (green), 2 (red), and 3 (blue) represent the clustered structures, where the structure from the original crystal structure (PDB code: 2Z0DB, black) is in the group 1. The representative alternative conformations (I and II) in group 2 and 3 are colored in cyan and yellow, correspondingly.

FIGS. 8A to 8C are the three conformations of LC3B summarized from the ensemble structures and used for ensemble docking. FIG. 8A is the original structure from the crystal structure (PDB code: 2Z0DB). FIG. 8B is alternative conformation I. FIG. 8C is alternative conformation II.

FIGS. 8D and 8E are the superposition of the three conformations of LC3B. The color schemes are the same as those in FIGS. 8A to 8C. The region in the dash circle of FIG. 8D is the ATG4B N-terminal tail binding site where the interaction induces the LC3B-mediated intermolecular allosteric regulation. An apparent change of the side-chain orientation within this region are observed among the three conformations. In addition, the C-terminal tail (the region in the dash circle of FIG. 8E) also shows huge conformational difference. The overall root-mean-square deviation (RMSD) between the original structure from the crystal structure and alternative conformation I and alternative conformation II are 3.38 Å and 2.77 Å, respectively. The RMSD between alternative conformation I and alternative conformation II is 2.79 Å.

FIGS. 9A and 9B illustrate the desired region of pose location. To quantify the extent of the pose located in the allosteric site, the sum of the distance between the drug center of mass (red ball) to the closest heavy atom (yellow) from the residues (51-54 and 58-59) of LC3B (green cartoon) and the distance between the drug center of mass to the closest heavy atom (orange) from the residues (5-14) of ATG4B N-terminal (cyan cartoon) was measured. It is showed in FIG. 9A a docked drug (sticks) of which the drug center of mass was within the desired location (the transparent region colored in red in the zoom in view of FIG. 9B) for the illustration.

FIG. 10 is a scheme of the fluorogenic 3CL^{pro} protease enzymatic assay. The fluorescence of Edans in the C-terminal of the peptide (SEQ ID NO: 15) substrate (Dabcyl-KTSAVLQSGFRKME-Edans) is quenched by the Dabcyl in the N-terminus without the 3CL^{pro} enzymatic cleavage. After the protease is added and cleaves the peptide substrate, the quencher molecule Dabcyl can no longer quench the fluorophore Edans which results in an increase in fluorescence emission at 460 nm. The intensity of this fluorescence is proportional to the protease activity.

FIG. 11 illustrates the cell viability assay on the colorectal cancer cell line, HCT116, for 27 commercially available drugs out of 48 top-ranked drugs that could play as ATG4B-LC3B recognition interface blocker suggested from the virtual drug screening. To examine the underlining mechanism of drugs contributing to the suppression of cell viability resulted from the autophagy pathway, the drugs were tested on both normal cancer cells (shCtrl/HCT116) and ATG4B silencing cancer cells (shATG4B/HCT116), between which the drug suppression effects were compared. Drugs showed reduced suppression in shATG4B/HCT116 compared to shCtrl/HCT116 suggested their suppression effect that might result from the perturbation of the autophagy pathway.

FIG. 12A illustrates the superposed HSQC spectra of LC3B-alone (colored in red) and LC3B titrated with vinorelbine with a 1:1 ratio (colored in blue). The residues showing chemical shift perturbation are highlighted in rectangles.

FIG. 12B illustrates LC3B structure (cartoon, colored in grey) with the perturbed residues (sticks) highlighted in red in the presence of vinorelbine.

FIGS. 13A to 13E show that vinorelbine has an additive and synergistic effect on the cancer cell viability and attenuates the autophagy flux by tioconazole co-treatment (FIGS. 13A and 13B). The cytotoxicity of vinorelbine (Vb) on the colorectal cancer cell line HCT116 without (shCtrl/HCT116, FIG. 13A) or with ATG4B silencing (shATG4B/HCT116, FIG. 13B) is shown. While vinorelbine showed its cytotoxicity in shCtrl/HCT116, its effect was reduced in shATG4B/HCT116, which suggested that the cytotoxicity of vinorelbine was ATG4B dependent. Tc refers to tioconazole, a known drug that can inhibit ATG4B activity and sensitize the cancer cell to chemotherapy, and was used as a positive control. DXMS refers to dexamethasone, a drug that showed no inhibitory effect on ATG4B, and was used as a negative control. DMSO: one of the solvents used to dilute the drug concentration. Under 0.8% DMSO, which is the concentration that is higher than those used to prepare all the different concentrations of drugs, it only showed a minor effect on cancer cell viability and suggested that the observed cytotoxicity of drugs was mainly contributed by drugs themselves (FIGS. 13C and 13D). The cytotoxicity of vinorelbine with tioconazole co-treatment in pancreatic cancer cell lines, AK4.4 (FIG. 13C), and AsPC-1 (FIG. 13D). Vinorelbine (Vb) and tioconazole (Tc) showed an additive effect in AK4.4 and even a synergistic effect in AsPC-1 (FIG. 13E). The level of LC3B-II was quantified by western blotting for AK4.4 and AsPC-1 treated with vinorelbine only (FIG. 13E). The increased level of LC3B-II indicated the attenuation of autophagy flux due to the inhibition of ATG4B by vinorelbine and tioconazole.

FIGS. 14A to 14D show that vinorelbine (Vb) and tioconazole (Tc) alone or tioconazole combined with vinorelbine or with designed peptides for targeting LIR binding pocket on LC3B, i.e., TAT-N-term-7 (tat-N7 or t-N7, with an amino acid sequence of SEQ ID NO: 16 (YGRKKRRQRRR-GGS-YDTLGIF)) and TAT-N-term-9 (tat-N9 or t-N9, with an amino acid sequence of SEQ ID NO: 17 (YGRKKRRQRRR-GGS-YDTLRFAEF)), can suppress xenograft mouse tumor. FIG. 14A illustrates the timeline of the in vivo treatment for the pancreatic ductal adenocarcinoma (PDAC) xenograft mouse. 5, 10 or 20 mg/kg vinorelbine was treated to the mouse via pre-oral (P.O.) administration or intraperitoneal (I.P.) injection at day 3, 5, 7, 10, 12, and 14. In addition, 10 mg/kg tat-N7, 10 mg/kg tat-N9 or 20 mg/kg tioconazole were treated to the mouse via intraperitoneal (I.P.) injection. The mouse was sacrificed at day 16, and the tumor size and the mouse body weights were measured. FIG. 14B shows the mouse tumor size measured on day 16. The tumor was effectively suppressed by vinorelbine when treated with a dose of 10 mg/kg or by tioconazole with a dose of 20 mg/kg, which resulted in less than half of the tumor size in the control group. FIG. 14C shows the mouse body weight measured on day 16. No obvious difference in the body weight was observed between the drug-treated groups and the control group, suggesting that vinorelbine alone, tioconazole alone, or tioconazole combined with vinorelbine, TAT-N-term-7 or TAT-N-term-9 had no apparent side effect under the used concentration. FIG. 14D shows the LC3B-II level in the tumor tissue quantified using western blotting. LC3B-II was found to be diminished under the treatment of vinorelbine, implying that tumor suppression and its underlining regulation of autophagy due to vinorelbine treatment may differ from that in the cell level.

FIGS. 15A to 15E show the relative organ weight (the organ weight of the subject divided by the body weight thereof) of FBV mice after the treatment by tioconazole (Tc), TAT-N-term-9 (Tat-N9), TAT-N-term-7 (Tat-N7), Tc combined with Tat-N9 (Tc+Tat-N9), or Tc combined with Tat-N7 (Tc+Tat-N7). The relative lung ratio, heart ratio, liver ratio, spleen ratio, and kidney ratio of the 6 treatment groups are shown in FIGS. 15A to 15E, respectively. Vehicle: 2.5% DMSO-containing buffer without any dissolving drugs; Tc: 20 mg/kg Tc; Tat-N9: 10 mg/kg Tat-N9; Tc+Tat-N9: 20 mg/kg Tc+10 mg/kg Tat-N9; Tat-N7: 10 mg/kg Tat-N7; and Tc+Tat-N7: 20 mg/kg Tc+10 mg/kg Tat-N7.

FIG. 16A illustrates the flowchart of the computational workflow of ensemble docking to identify possibly repurposed allosteric drugs of ATG4B for viability assays on cancer cell lines. Ensemble structures of LC3B including homologous crystal structures and sampled conformation by MD simulations were clustered and summarized into three major conformations in the first two PCs space, including the one in the original crystal structure (PDB code: 2Z0DB) and two alternative conformations. The three conformations were used to dock 2016 FDA-approved drugs and, with two designed ranking methods, i.e., normalized ranking and logarithm of odds (LOD) scoring, resulted in six ranked drug lists. 19 top-ranked drugs picked by five selection rules were tested for the inhibition of the viability on cancer cell lines, HCT116 and AsPC-1.

FIG. 16B illustrates the cell viability assays for selected ATG4B allosteric drugs. Drugs with indicated concentration were treated on HCT116 (top) and AsPC-1 (bottom) cancer cell lines for three repeats. The measured cell viability was normalized by the corresponding blank control. Ponatinib, moxidectin, aclacinomycin A, ethynyl estradiol, temsirolimus, Evans blue, and netupitant showed considerable inhibition (<60% cell viability) in either or both cell lines. Tioconazole, a known ATG4B inhibitor that competed the catalytic site with the substrate, served as the positive control. Dexamethasone, a known drug without any inhibition effect on ATG4B suggested from both previous computational and experimental screening, served as the negative control. The significance of the reduced cell viability compared to the blank control was tested using the Student's t-test; * p<0.05, ** p<0.01, *** p<0.001. Error bars denote S.E.M.

FIG. 17 illustrates the inhibition of the selected ATG4B allosteric drugs on the cell viability of HCT116 cell lines of the ATG4B-silencing strain (shATG4B/HCT116 strain, S strain, top) and the control strain (shCtrl/HCT116, C strain, bottom). Drugs were treated on the cells with indicated concentration. Each assay was repeated in three times. The measured cell viability was normalized by the corresponding blank control. Tioconazole, the positive control, a known drug that could inhibit ATG4B. Dexamethasone, the negative control, a known drug with no inhibition effect on ATG4B. The significance of the difference of the cell viability inhibitions of a drug between the S strain and C strain was tested using the Student's t-test; * p<0.05, ** p<0.01, *** p<0.001. Ponatinib, dolutegravir, ledipasvir, paritaprevir, aclacinomycin A, ethynyl estradiol, and emsirolimus showed significant differences (p-value <0.05) of cell viability inhibition between the S strain and C strain, suggesting that the inhibition was partially contributed from the perturbation of the autophagy pathway. Error bars denote S.E.M.

FIG. 18A shows the superposed HSQC spectra of LC3B-alone (colored in red) and LC3B titrated with moxidectin with a 1:1 ratio (colored in green). The residues showing chemical shift perturbation are highlighted in rectangles.

FIG. 18B illustrates the LC3B structure (cartoon, colored in grey) with the perturbed residues (sticks) highlighted in red in the presence of moxidectin.

FIG. 18C shows the superposed HSQC spectra of LC3B-alone (colored in red) and LC3B titrated with ponatinib with a 1:1 ratio (colored in blue). The residues showing chemical shift perturbation are highlighted in rectangles.

FIG. 18D illustrates the LC3B structure (cartoon, colored in grey) with the perturbed residues (sticks) highlighted in red in the presence of ponatinib.

FIG. 19 indicates the inhibition of ponatinib (Pn) and moxidectin (Mx) on the viability of cancer cells. To evaluate the inhibition of the cleavage by ponatinib and moxidectin, in vitro cleavage assays with a reconstructed, N-terminal C-Myc-attached LC3B were performed to quantify the catalysis of ATG4B. Ponatinib and moxidectin at designated concentrations showed effective inhibition of the cleavage and resulted in the unprocessed form of reconstructed LC3B.

FIGS. 20A to 20E indicate the inhibition of cancer cell line, AK4.4 and AsPC-1, viability by ponatinib (alone or combined with tioconazole) (FIG. 20A) and moxidectin (alone or combined with tioconazole (Tc) or vinorelbine (Vb)) (FIGS. 20B to 20E) with designated concentrations. Both treatments of ponatinib and moxidectin alone showed reduced cell viability, and the inhibition can be further enhanced by the combined treatment of tioconazole or vinorelbine.

FIGS. 21A to 21D illustrate in silico designed peptides for targeting LIR binding pocket on LC3B. FIG. 21A is the illustration of the designed peptides. N-term-18 was taken from the full length of ATG4B N-terminal tail (residue IDs: 1-18). N-term-9 was extracted from the segment that formed a close interaction with N-LC3B as suggested from the crystallized structures and MD simulations (residue IDs: 8-16). N-term-9 was later modified by replacing the fifth to eighth residues with a GG linker and then point-mutated to generate 134 different peptides. The binding affinity of the generated peptides was then evaluated by docking and MD simulations. The peptide, N-term-7, forming stable binding with LC3B observed in 100 ns MD simulations, was then chosen for, together with N-term-18 and N-term-9, testing the inhibition of ATG4B activity. FIG. 21B illustrates the first (left) and last snapshot (right) of the 30 ns simulation of the N-term-12 (TLTYDTLRFAEF) (SEQ ID NO: 18) interacted with LC3B (blue). The black letters denote the types of amino acid, and the green letters denote the N-terminus and C-terminus. The yellow circle indicates that the first three residues of N-terminus detached from and formed fewer contacts with LC3B. The last fifth to second residues (RFAE) close to the C-terminus formed fewer contacts (average contacts <1.0, referring to Table 5) with LC3B than other residues and appeared more flexible (RMSF >1 Å, referring to Table 5) in the simulation. FIG. 21C shows the 2 ns and 100 ns snapshots of the 100 ns simulation of the N-term-7 peptide (SEQ ID NO: 2 (YDTLGIF), green) interacting with LC3B (blue). FIG. 21D is the Cα root mean square deviation (RMSD) of YDTL and F in the mutant peptides (SEQ ID NO: 2 (YDTLGIF), SEQ ID NO: 14 (YDTLGGF), and SEQ ID NO: 3 (YDTLYGF)) selected from the docking screening, and the template peptide (SEQ ID NO: 14 (YDTLGGF)), from those in the N-term-12 (SEQ ID NO: 18 (TLTYDTLRFAEF)) solved in the crystal structure. The N-term-7 peptide (SEQ ID NO: 2 (YDTLGIF), blue) was shown to be a stable one to bind with LC3B and resembled the docking pose of the N-term-12.

FIGS. 22A to 22D illustrate that peptides designed for competing LC3B with the ATG4B N-terminal tail showed moderate inhibition of ATG4B activity. FIG. 22A is western blots of the ATG4B cleavage assay. The in silico designed peptide N-term-7 (N7, Ac-YDTLGIF-NH₂ (Ac-SEQ ID NO: 2 —NH₂)) showed moderate and improved inhibition to ATG4B compared with the peptide derived from ATG4B N-terminal tail (N-term-18 or N18, SEQ ID NO: 19 (MDAATLTYDTLRFAEFED)). The band for the S-tag indicates the full-length, unprocessed form of recombinant pro-LC3B. N: the negative control, pro-LC3B only, which resulted in dark, unprocessed bands on the gel. P: positive control, pro-LC3 mixed with ATG4B, and the cleavage by ATG4B resulting in the lighter band for S-tag. Numbers indicated the normalized band intensities (divided by the negative control) for S-tag. FIG. 22B is quantification of the ATG4B activity derived from FIG. 22A and also shows the effect of peptide N-term-9 (N9, Ac-YDTLRFAEF-NH₂ (Ac-SEQ ID NO: 20 —NH₂)) on inhibition to ATG4B. The ATG4B activity was calculated as: (the band intensity of negative control (N)— the band intensity of measure)/(the band intensity of negative control (N)— the band intensity of positive control (P))×100% for the ATG4B cleavage assays and ((average RFU)/(average RFU without peptides))×100% for the ATG4B activity reporter assays. The results suggested that all the designed peptides can moderately inhibit the ATG4B activity, with the N-term-7 having the most inhibition effect. FIGS. 22C and 22D show the three-dimensional structure information when N-term 7 and N-term 9 targeting LIR binding pocket on LC3B, respectively.

FIGS. 23A to 23D indicate the NMR chemical shift perturbation of designed peptides titrated on LC3B. Figures showed the superposition of HSQC spectra and the perturbed residues mapped on the LC3B structure (right) for N-term-7 (FIG. 23A), N-term-18 (FIG. 23B), TAT-N-term-9 (FIG. 23C), and TAT-Ctrl (FIG. 23D). Left: the superposition of HSQC spectra (left) of LC3B with (blue or green) and without the titrated peptides (red). The perturbed residues were highlighted in rectangles in the HSQC spectra. Right: the perturbed residues mapped on the LC3B structure. LC3B was shown in cartoon and colored in blue except for those residues contacting the fourth to seventh (YDTL) and last (F) residues of N-term-12 in the crystal structure, which were colored in red. The perturbed residues were shown in sticks-and-balls. The docked peptides were shown in cartoons and colored in green, where “N” and “C” indicated the N-terminus and C-terminus. The perturbed residues overlapping with those residues involved in the native contacts with N-term-12 (YDTL and F) were labeled. For N-term-7 (FIG. 23A), the docking pose of the N-term-7 (green) and LC3B from the last snapshot of a 100 ns MD simulation was shown. The perturbed sites by N-term-7 were exactly located on the desired LIR binding pocket on LC3B. For N-term-18 (FIG. 23B), the docking pose of the N-term-12 and the LC3B structure was taken from the last snapshot of a 30 ns MD simulation of the binding between LC3B and N-term-12 (green), which was the segment of N-term-18 solved in the crystal structure and having more interaction with LC3B. The perturbed residues were overlapped with N-term-12's binding site. For TAT-N-term-9 (FIG. 23C) and TAT-Ctrl (FIG. 23D), the peptides and LC3B structures were also taken from the same 30 ns MD simulation as in FIG. 23B), except that showed the N-term-9 part from the N-term-12. The pattern of perturbed residues for TAT-N-term-9 was similar to those observed in N-term-7 and N-term-18 and overlapped with the binding site of N-term-9. The perturbation pattern was mainly contributed by N-term-9 since TAT-Ctrl gave a different perturbation pattern and less perturbing the N-term-9 binding site.

FIGS. 24A and 24B show that the peptides served as the ATG4B allosteric inhibitors can suppress the survival of cancer cells. FIG. 24A shows the inhibition of N-term-7 (N7) and N-term-9 (N9) on the viability of colorectal cancer cell line, HCT116, examined by the cell viability assays. To increase the cell penetration of the peptides, a cell-penetrating peptide, TAT, followed by a linker, “GGS,” was prepended to the peptides, resulting in TAT-N-term-7 (tat-N7 or t-N7, with an amino acid sequence of SEQ ID NO: 16 (YGRKKRRQRRR-GGS-YDTLGIF)) and TAT-N-term-9 (tat-N9 or t-N9, with an amino acid sequence of SEQ ID NO: 17 (YGRKKRRQRRR-GGS-YDTLRFAEF)). Both peptides showed moderate inhibition of the cancer cell viability (the sixth to ninth bars), which did not result from the introduced “TAT”-GGS sequence (TAT-Ctrl) (the fourth and fifth bars), suggesting the validity of designing allosteric drugs of ATG4B for cancer therapy. When co-treated with the tioconazole (Tc), a known drug that can suppress the cancer cell viability by targeting the ATG4B active site, both TAT-N-term-7 and TAT-N-term-9 showed synergistic effects that sensitized the cancer cells to Tc (the last fifth and sixth bars) compared to treatment with Tc only (the second and third bars). FIG. 24B indicates the inhibition of N-term-7 (N7) and N-term-9 (N9) on the viability of pancreatic cancer cell line, AsPC-1. N-term-9 showed an inhibition of the cell viability (the last three bars) that is comparable to Tc (the second and third bars). N-term-7 only showed slight inhibition on the cell viability where the effect was apparent when the concentration of 40 μM was treated (the last fourth bars). The significance of the viability differences was calculated using t-test. * p<0.05; ** p<0.01; *** p<0.001.

FIGS. 25A to 25G indicate the Coarse-Grained Connection Matrix (CGCM) for ATG4B and the clustered ICCs that suggest an allosteric site. FIG. 25A shows that the coarse-grained communication matrix is over the average perturbing the 41 Cα atom within 8 Å of the Cys74. FIG. 25B shows that the td-LRT-derived 10 ICCs (in red spheres) and their corresponding communication scores in ATG4B are (TRP27, 0.71), (TYR33, 0.71), (ARG31, 0.70), (LYS39, 0.7), (LYS32, 0.69), (ILE28, 0.67), (ARG49, 0.67), (LEU6, 0.67), (ARG12, 0.67), and (TYR8, 0.67). FIG. 25C indicates that the top two docking results are subject to further MD analyses including MM/GBSA-based affinity evaluation and pose-site distance characterization; in the pose-site distance column, the distances from docking and after 10 ns simulation are outside and inside the parentheses, respectively. FIG. 25D indicates a representative snapshot of how moxidectin (Mx; in ball-and-stick) interacts with the allosteric residues (in yellow sphere) that are within 4 Å of the Mx. FIG. 25E shows that inhibition % is defined as (S-tag-blotting intensity of the sample minus that of drug-free ATG4B+LC3B mixture)/(S-tag-blotting intensity of the LC3B only minus that of drug-free ATG4B+LC3B mixture). The higher the inhibition percentage, the better the drug. FIG. 25F shows that HCT116 colorectal, AsPc-1 pancreatic and MAD-MB-468 breast cancer cell lines are suppressed by 10 μM of the top 2 FDA drugs screened from small-molecule docking and MD simulations. FIG. 25G shows that 4 μM Mx co-used with 500 nM tioconazole, an ATG4B active site inhibitor (Liu et al., 2018), shows an improved tumor suppression efficacy as compared with individual treatment alone (p-value <0.01, Student's t-test, two tailed).

FIG. 26 shows the homodimer structure of 3CL^{pro}. The N-terminus (residues 1-9), domain I (residues 10-98), domain II (residues 100-181), and domain III (residues 198-302) are shown in thickened red tube, orange, blue and ice blue, respectively. One of the monomers in the front is shown in transparent white. The catalytic residues HIS41 and CYS145 are represented in a ball-and-stick model.

FIGS. 27(A) and 27(B) illustrate that norvancomycin contacts with N-terminus interface residues (orange color). FIG. 27(A) indicates MD snapshot at 0 ns, and FIG. 27(B) indicates MD snapshot at 10 ns. The blue and red colors indicate the domain II interface residues (SER121, PRO122, SER123, GLY124, VAL125, SER139, PHE140, LEU141 and GLU166) and N-terminus interface residues (SERI, GLY2, ARG4, METE, ALAI and PROS), respectively, and the active site residues are shown in a ball-and-stick model.

FIG. 28 shows the inhibition percentage of different FDA drugs in single use or in combination. First, the interface blocker norvancomycin was tested for the inhibition ability in the in vitro 3CL^{pro} assay. Inhibition percentage is calculated by “1−(Iₜₑₛₜ−I_{substrate})/(Iₚₒₛᵢₜᵢᵥₑ−I_{substrate}),” where I represents the value of inhibition ability. The data of 6 to 25 μM single use of norvancomycin and 12.5 to 50 μM in combination with the active-site inhibitor boceprevir are shown by the average value of two replicates.

### DETAILED DESCRIPTION

In the following description of the embodiments, reference is made to the accompanying drawings, which form a part thereof, and within which are shown by way of illustrative embodiments by which the disclosure may be practiced. It is to be understood that other embodiments may also be utilized and structural changes may be made without departing from the scope of the disclosure.

As used in this specification and the appended claims, the singular forms “a,” “an,” and “the” include plural referents unless the content clearly dictates otherwise. As used herein, the term “and” is intended to be inclusive unless otherwise indicated. As used herein, the term “or” is generally employed in its sense including “and/or” unless the context clearly dictates otherwise.

As used herein, the term “about” refers to a degree of deviation for a property, composition, amount, value or parameter as identified, such as deviations based on experimental errors, measurement errors, approximation errors, calculation errors, standard deviations from a mean value, routine minor adjustments, and so forth.

As used herein, the terms “comprising,” “having,” “including,” and “containing” are to be construed as open-ended terms (i.e., meaning “including, but not limited to”) unless otherwise noted.

As used herein, the terms “composition” and “composite” are used interchangeably. As used herein, the term “ensemble structure” is well-known in the structural determination by nuclear magnetic resonance (NMR), the ensemble of structures rather than a single structure, with perhaps several members, all of which fit the NMR data and retain good stereochemistry, is deposited with the Protein Data Bank. Comparisons between the models in this ensemble provide some information on how well the protein conformation is determined by the NMR constraints. It should be noted that all the sequences corresponding to NMR-determined ensemble structures have the same sequences (one protein with variable conformations). The structural ensemble here, additionally, refers to different proteins with variations in sequence and/or length but having similar main chain conformations, in addition to those structures, such as from NMR determinations or from molecular dynamics simulations, having the same sequence but differing in structure due to natural shape fluctuations.

### Materials and Methods

Identification of ATG4B-LC3 recognition interfaces

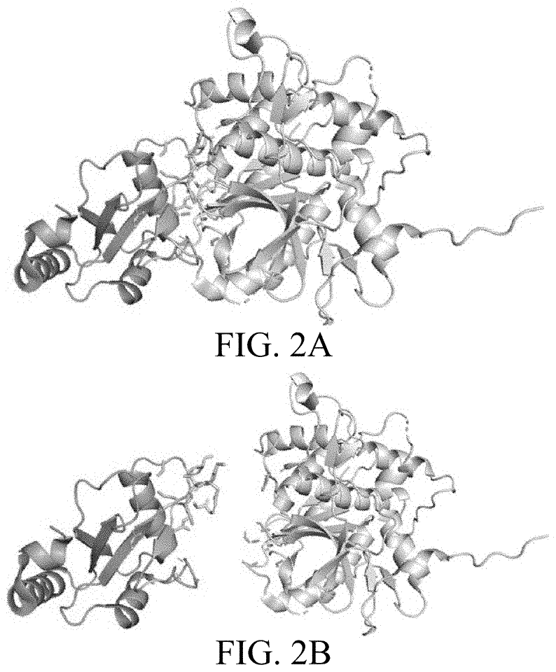

The ATG4B-LC3 recognition interfaces were defined based on the crystallized complex structure deposited under the PDB code of 2Z0D. By defining the contact as two heavy atoms that were within 4 Å, the recognition interface was described as those heavy atoms that formed the intermolecular contacts between ATG4B and LC3B as shown in FIGS. 2A and 2B. The resulting interface for ATG4B composed of heavy atoms from Tyr54, Gly71, Trp72, Gly73, Cys74, Met75, Lys137, Trp142, Tyr143, Gly144, Pro145, Asn146, Gln150, Lys154, Met170, Asp171, Asn172, Thr173, Pro227, Leu228, Arg229, Gly231, Leu232, Thr233, Tyr239, Lys259, Pro260, Ser262, Ala263, Asp314, Ser316, Ile317, Pro347, and Glu350. The resulting interface for LC3B composed of heavy atoms from Tyr38, Gln43, Ile64, Arg68, Ala75, Asn76, Gln77, Ala78, Phe79, Phe80, Leu82, Gly85, His86, Ser87, Va189, Ser90, Va191, Ala114, Ser115, Gln116, Glu117, Thr118, Phe119, and Gly120.

### In Silico Drug Screening for ATG4B-LC3 Recognition Interfaces

In silico drug screening for 2016 FDA-approved drugs on both ATG4B and LC3 interfaces was performed by molecular docking and molecular dynamics (MD) simulations. 2016 FDA-approved drugs were obtained from MedChemExpress (MCE), and the protonation state of which was determined at pH 7 by the Chemicalize (Swain, 2012, J. Chem. Inf. Model. 52, 613-615). The missing residues of ATG4B and LC3 were modeled by SWISS-MODEL (Kiefer et al., 2009, Nucleic Acids Res. 37, D387-92) using the existing x-ray crystal structure (PDB ID: 2Z0D) as template.

For each FDA-approved drug, two sets of docking were performed by Vina (Trott O. and Olson A. J., 2010, J. Comput. Chem. 31, 455-461)— one on ATG4B and the other on LC3. 20 docking poses of each ligand-receptor (FDA drug and ATG4B/LC3) pair were clustered by hierarchical clustering (Zepeda-Mendoza et al., 2013, Encyclopedia of Systems Biology 43, 886-887) using the pairwise RMSD of the poses as the distance. The interface residues on ATG4B and LC3 were defined as the residues contacting any heavy atom from the other protein within 4 Å, and the contact number of a drug pose was defined as the number of heavy atoms from the interface residues staying within 4 Å from the pose.

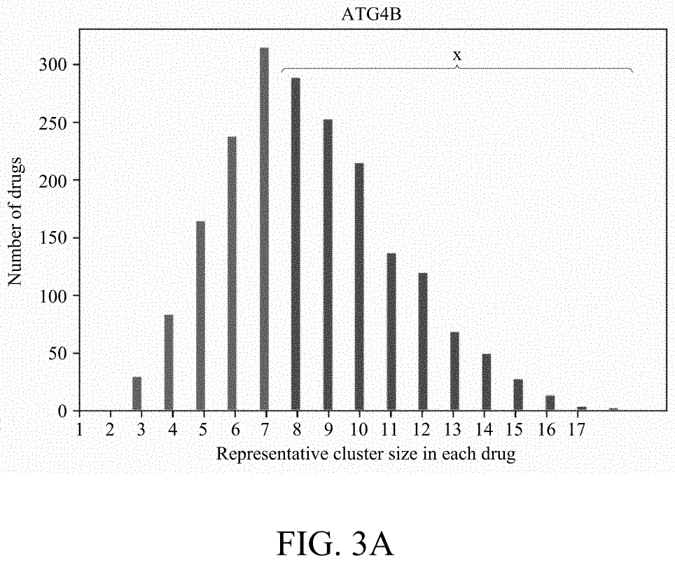

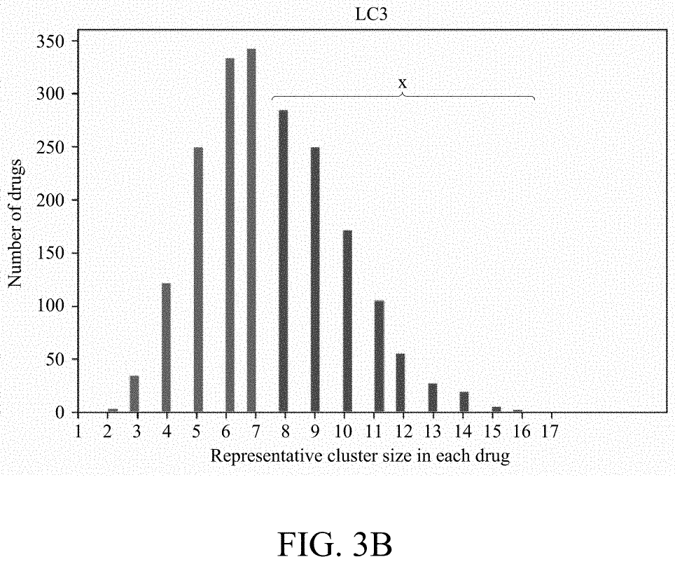

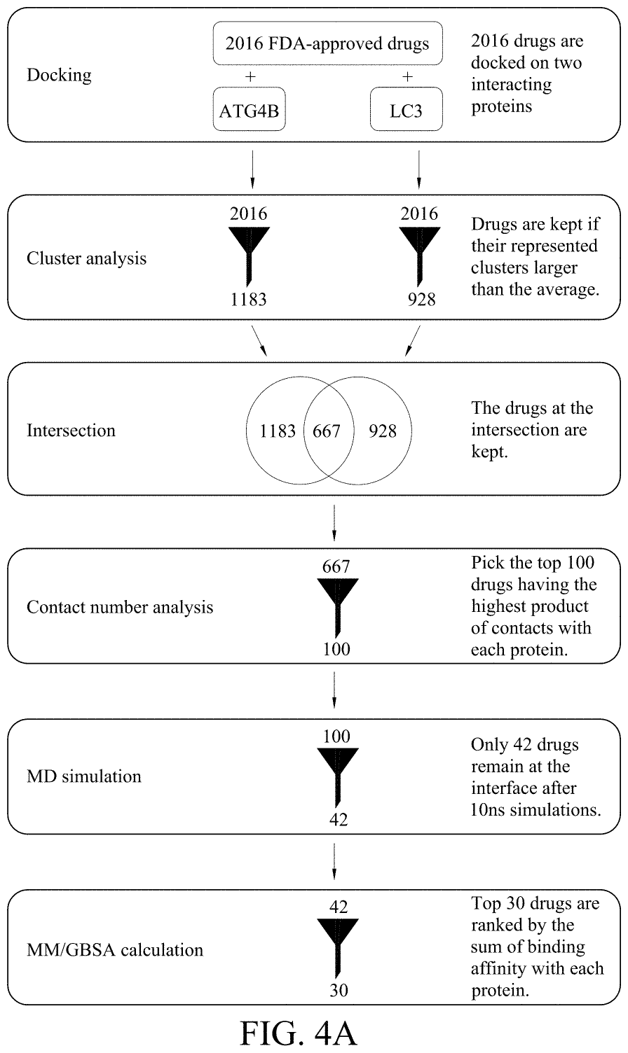

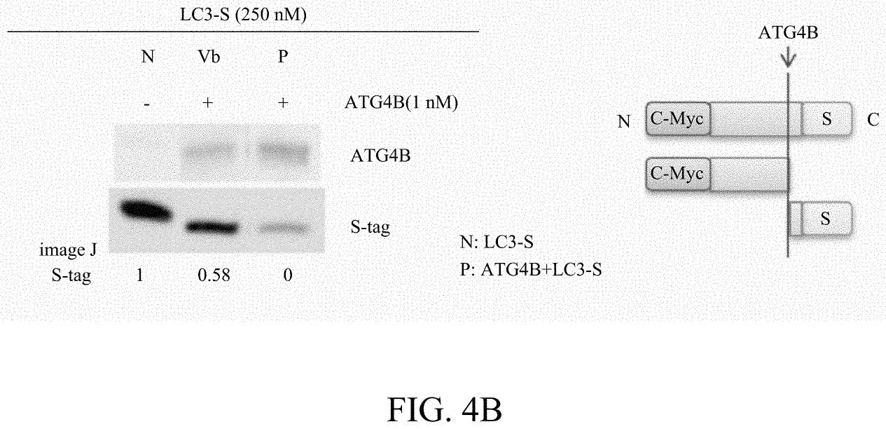

A cluster that contains many docking poses indicates a higher conformational entropy of the drug at its bound state, which favors the binding free energy because of a lessened decrease of entropy upon drug-protein complexation. Every drug had its largest cluster of poses from the docking, which served as the representative cluster for the drug. Only those drugs whose representative cluster was larger (had more poses) than the average size of the representative clusters of all the drugs were kept (FIGS. 3A and 3B), and passed to the next stage in the prioritizing process of drug candidates for repurposing. There were 667 drugs left (FIG. 4A).

The drugs that remained in both ATG4B's and LC3's docking runs were subject to further analysis. As discussed above, contact number can roughly suggest pairwise atom interactions. Assuming a small interface where all the interface residues in ATG4B interact with all the interface residues in LC3, the product (multiplication) of a drug's contact with both proteins, rather than the sum of the two, is more indicative on how many pairwise atomic interactions are blocked by the drug. Here, the contact number of a drug to a protein's interface residues was calculated from the average contact number of all the poses of the largest cluster for the drug. Out of 667, the top 100 drugs having the largest products of contacts with both proteins were selected for the following MD simulations on both protein-drug complexes and binding free energy calculations by MM/GBSA.

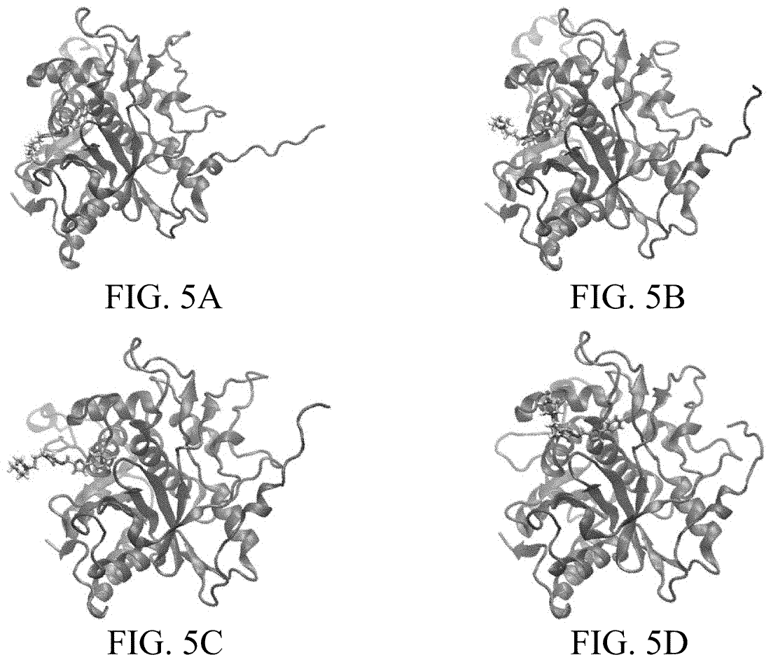

The simulation package OpenMM (Eastman et al., 2017, PLoS Comput Biol. 13, e1005659) with AMBER ff14SB force field (Maier et al., 2015, J. Chem. Theory Comput. 11, 3696-3713) was used to examine the binding stability for the top-ranked 100 drugs on both ATG4B and LC3 interfaces, and the results were reported in Table 1 below. The drugs that left the interface within 10 ns were discarded (FIGS. 5A to 5D), and those that stayed were rank-ordered based on the sum of their binding free energy, calculated from MM/GBSA, on both proteins (Table 2).

TABLE 1

| | Drug | Contact | Contact Ratio³ | |
|---|---|---|---|---|
| Rank¹ | Name | Number² | ATG4B | LC3 |
| 1 | Ritonavir | 2129.7 | 1.13 | [symbol] |
| 2 | Dronedarone | 1971.0 | 1.57 | 0.50 |
| 3 | Saquinavir | 1942.1 | 0.54 | 0.34 |
| 4 | Manidipine | 1821.1 | 0.35 | 0.44 |
| 5 | Nintedanib | 1656.3 | 1.45 | 0.33 |
| 6 | Niguldipine | 1596.0 | 0.37 | [symbol] |
| 7 | Paclitaxel | 1592.5 | [symbol] | [symbol] |
| 8 | Telotristat | 1512.0 | 0.78 | 0.35 |
| | ethyl | | | |
| 9 | Selexipag | 1468.9 | 1.05 | [symbol] |
| 10 | Enasidenib | 1448.9 | 0.41 | 0.43 |
| 11 | Elagolix | 1446.6 | 1.08 | 0.32 |
| 12 | Sofosbuvir | 1442.0 | 1.09 | [symbol] |
| 13 | Lorglumide | 1397.3 | 0.47 | [symbol] |
| 14 | Fumagillin | 1394.3 | 0.91 | 0.34 |
| 15 | Almitrine | 1393.9 | 0.73 | [symbol] |
| 16 | Sarpogrelate | 1381.8 | 0.94 | [symbol] |
| 17 | Azimilide | 1367.6 | [symbol] | 0.43 |
| 18 | Dipyridamole | 1360.0 | 1.41 | [symbol] |
| 19 | Amprenavir | 1356.4 | 1.50 | 0.62 |
| 20 | Eltrombopag | 1350.0 | 1.31 | [symbol] |
| 21 | Temocapril | 1337.1 | 1.37 | [symbol] |
| 22 | Ranolazine | 1335.0 | [symbol] | [symbol] |
| 23 | Vilanterol | 1321.6 | 1.23 | [symbol] |
| 24 | Sunitinib | 1311.0 | 1.02 | [symbol] |
| 25 | Sacubitril | 1306.8 | 1.15 | 0.69 |
| 26 | Dabrafenib | 1305.0 | 0.86 | 0.32 |
| 27 | Quinapril | 1299.0 | 0.81 | [symbol] |
| 28 | Dacomitinib | 1297.8 | [symbol] | 0.41 |
| 29 | 383365-04-6 | 1293.6 | 0.98 | [symbol] |
| 30 | Benfotiamine | 1288.6 | 1.70 | 0.35 |
| 31 | Avanafil | 1283.4 | 1.38 | 0.44 |
| 32 | Aprepitant | 1269.6 | 0.53 | [symbol] |
| 33 | Pimozide | 1251.3 | 1.51 | 0.58 |
| 34 | Latanoprost | 1239.0 | 1.38 | 0.31 |
| 35 | Bosentan | 1238.3 | 0.85 | 0.35 |
| 36 | Doravirine | 1230.0 | 0.56 | [symbol] |
| 37 | Lisinopril | 1220.0 | 1.19 | [symbol] |
| 38 | Dapt | 1215.2 | 1.13 | [symbol] |
| 39 | 15(s)-Fluprostenol | 1211.0 | 0.35 | [symbol] |
| | isopropyl ester | | | |
| 40 | Racecadotril | 1210.7 | 1.41 | [symbol] |
| 41 | Rolapitant | 1206.8 | 0.51 | [symbol] |
| 42 | Ramipril | 1206.1 | 1.32 | [symbol] |
| 43 | Ezetimibe | 1200.9 | 0.51 | 0.38 |
| 44 | Afatinib | 1198.8 | 1.41 | 0.34 |
| 45 | Imidapril | 1197.9 | 1.54 | 0.32 |
| 46 | Enalapril | 1191.7 | 0.50 | [symbol] |
| 47 | Nafronyl | 1179.0 | 0.93 | [symbol] |
| 48 | Penfluridol | 1178.5 | [symbol] | 0.62 |
| 49 | Rilpivirine | 1161.5 | 0.63 | [symbol] |
| 50 | Astemizole | 1158.5 | 0.69 | 0.33 |
| 51 | Indacaterol | 1157.3 | 1.07 | [symbol] |
| 52 | 26652-09-5 | 1153.8 | 0.51 | [symbol] |
| 53 | Loperamide | 1150.9 | 0.63 | 0.64 |
| 54 | Misoprostol | 1150.8 | 1.09 | 0.33 |
| 55 | Vatalanib | 1137.7 | 1.36 | 0.27 |
| 56 | Hexetidine | 1134.6 | 1.47 | 0.54 |
| 57 | Fenoxaprop-p-ethyl | 1134.3 | 0.89 | 0.38 |
| 58 | Naftopidil | 1134.0 | 1.04 | 0.31 |
| 59 | Isavuconazole | 1130.2 | [symbol] | 0.32 |
| 60 | Lomerizine | 1126.5 | 0.98 | [symbol] |
| 61 | Vinorelbine | 1126.3 | 0.74 | 0.52 |
| 62 | Carbosulfan | 1125.0 | 0.93 | [symbol] |
| 63 | Bopindolol | 1122.8 | 1.18 | [symbol] |
| 64 | Riociguat | 1118.3 | 0.64 | 0.39 |
| 65 | Lasofoxifene | 1110.7 | [symbol] | [symbol] |
| 66 | Eliglustat | 1110.0 | 0.32 | 0.48 |
| 67 | Argatroban | 1107.8 | 1.24 | [symbol] |
| 68 | Miconazole | 1107.3 | [symbol] | [symbol] |
| 69 | Bepridil | 1104.4 | 1.08 | 0.36 |
| 70 | Labetalol | 1103.3 | 1.68 | [symbol] |
| 71 | Mizolastine | 1101.9 | 1.56 | 0.48 |
| 72 | Clevidipine | 1101.3 | 1.20 | [symbol] |
| 73 | Proglumide | 1098.1 | 0.37 | [symbol] |
| 74 | Oxiconazole | 1094.3 | 0.86 | 0.33 |
| 75 | Benazepril | 1092.0 | 1.11 | 0.36 |
| 76 | Apremilast | 1092.0 | 1.61 | 0.00 |
| 77 | Trifluperidol | 1089.1 | 1.02 | 0.39 |
| 78 | Cis-4,7,10,13,16,19- | 1088.7 | 0.74 | 0.30 |
| | docosahexaenoic acid | | | |
| 79 | Pravadoline | 1088.6 | 1.60 | [symbol] |
| 80 | Netupitant | 1087.3 | 0.97 | [symbol] |
| 81 | Lumacaftor | 1086.6 | 0.56 | [symbol] |
| 82 | Formoterol | 1086.6 | 1.89 | 0.33 |
| 83 | Sitagliptin | 1082.0 | [symbol] | 0.52 |
| 84 | Cefuroxime | 1080.2 | [symbol] | [symbol] |
| 85 | Cilostazol | 1077.4 | 1.61 | 0.59 |
| 86 | Fluspirilene | 1077.3 | 1.23 | 0.50 |
| 87 | Desmethyl | 1076.9 | 1.20 | 0.34 |
| | Erlotinib | | | |
| 88 | Nortriptyline | 1076.7 | 0.73 | [symbol] |
| 89 | Propafenone | 1072.7 | 1.14 | [symbol] |
| 90 | Oxatomide | 1071.1 | 1.41 | [symbol] |
| 91 | Isoconazole | 1068.4 | [symbol] | [symbol] |
| 92 | Terfenadine | 1066.8 | 2.00 | 0.48 |
| 93 | Acebutolol | 1061.5 | 1.11 | 0.71 |
| 94 | Butoconazole | 1061.1 | 0.86 | 0.31 |
| 95 | Rebamipide | 1060.0 | [symbol] | 0.48 |
| 96 | Famciclovir | 1058.2 | 1.78 | [symbol] |
| 97 | Lubiprostone | 1055.2 | 1.16 | [symbol] |
| 98 | Cilnidipine | 1053.3 | 0.79 | 0.42 |
| 99 | Bestatin | 1052.8 | 0.87 | [symbol] |
| 100 | Mitoxantrone | 1052.1 | 0.59 | [symbol] |

¹The ranking order is based on “Contact Number.”
²The total MM/GBSA is the sum of ATG4B and LC3 binding free energy.
³Contact ratio is the smallest value of simulation contact number divided by docking pose contact number. The contact ratio lower than 0.3 is considered as the drug has left the interface of protein, marked in italic and bold.

TABLE 2

| | |
|---|---|
| Drug | MM/GBSA (kcal/mole) |

| Rank¹ | Name | ATG4B | LC3 | Total² |
|---|---|---|---|---|
| 1 | Dronedarone | −47.2 | −43.1 | −90.3 |
| 2 | Pimozide | −45.4 | −39.4 | −84.8 |
| 3 | Hexetidine | −42.4 | −41.2 | −83.6 |
| 4 | Mizolastine | −47.5 | −33.1 | −80.6 |
| 5 | Nintedanib | −45.2 | −34.5 | −79.7 |
| 6 | Afatinib | −50.2 | −28.8 | −79.0 |
| 7 | Acebutolol | −40.9 | −37.5 | −78.4 |
| 8 | Loperamide | −32.3 | −42.8 | −75.1 |
| 9 | Terfenadine | −44.7 | −30.2 | −74.9 |
| 10 | Fluspirilene | −47.7 | −26.6 | −74.4 |
| 11 | Sacubitril | −33.4 | −34.0 | −67.4 |
| 12 | Cilostazol | −39.8 | −27.0 | −66.7 |
| 13 | Avanafil | −38.0 | −28.6 | −66.5 |
| 14 | Manidipine | −34.8 | −31.5 | −66.3 |
| 15 | Cilnidipine | −37.8 | −27.5 | −65.3 |
| 16 | Eliglustat | −31.9 | −33.2 | −65.1 |
| 17 | Amprenavir | −36.0 | −28.9 | −64.9 |
| 18 | Bepridil | −43.1 | −21.3 | −64.4 |
| 19 | Vinorelbine | −34.1 | −29.6 | −63.7 |
| 20 | Saquinavir | −39.3 | −23.5 | −62.8 |
| 21 | Elagolix | −37.8 | −24.8 | −62.6 |
| 22 | Benfotiamine | −36.4 | −25.3 | −61.7 |
| 23 | Astemizole | −31.8 | −29.7 | −61.5 |
| 24 | Oxiconazole | −35.8 | −24.6 | −60.4 |
| 25 | Naftopidil | −35.1 | −24.4 | −59.5 |
| 26 | Formoterol | −35.8 | −23.6 | −59.4 |
| 27 | Latanoprost | −38.3 | −20.7 | −59.1 |
| 28 | Telotristat | −36.0 | −22.6 | −58.6 |
| | ethyl | | | |
| 29 | Trifluperidol | −31.7 | −26.9 | −58.6 |
| 30 | Dabrafenib | −33.4 | −22.9 | −56.3 |

¹The ranking order is based on the total MM/GBSA.
²The total MM/GBSA is the sum of ATG4B and LC3 binding free energy.

### Protein Expression and Purification

The sequence of glutathione S-transferase-tagged LC3B (GST-LC3B) cloned into the pGEX-6p vector was kindly provided by Dr. Nobuo N Noda (Noda et al., 2008, Genes to Cells 13, 1211-1218). To express LC3B, the vector was transformed into the expression host E. coli BL21 (DE3). A single colony was picked and incubated overnight in 10 mL Luria broth (LB) medium containing 10 μL of 1 mM ampicillin at 37° C. The cultured medium was then mixed with 1 L LB medium (or M9 minimal medium containing ¹⁵NH₄Cl for ¹⁵N labeling) and continuously grown to reach an OD₆₀₀ of 0.6 to 0.7. The expression of GST-LC3B was induced by 1 mM isopropyl-O-D-thiogalactopyranoside (IPTG) at 25° C. for 10 hours and then stopped at 4° C. for another 20 minutes. The suspended bacterial cells were centrifuged, and the pellet was re-suspended in the 15 mL of binding buffer A containing 140 mM NaCl, 2.7 mM KCl, 10 mM NaHPO₄, 1.8 mM KH₂PO₄ at pH 7.3. The bacterial cells were broken by Sonicator (Qsonica Q125 Sonicator) in an ice bath and centrifuged. The supernatant containing GST-LC3B was then collected.

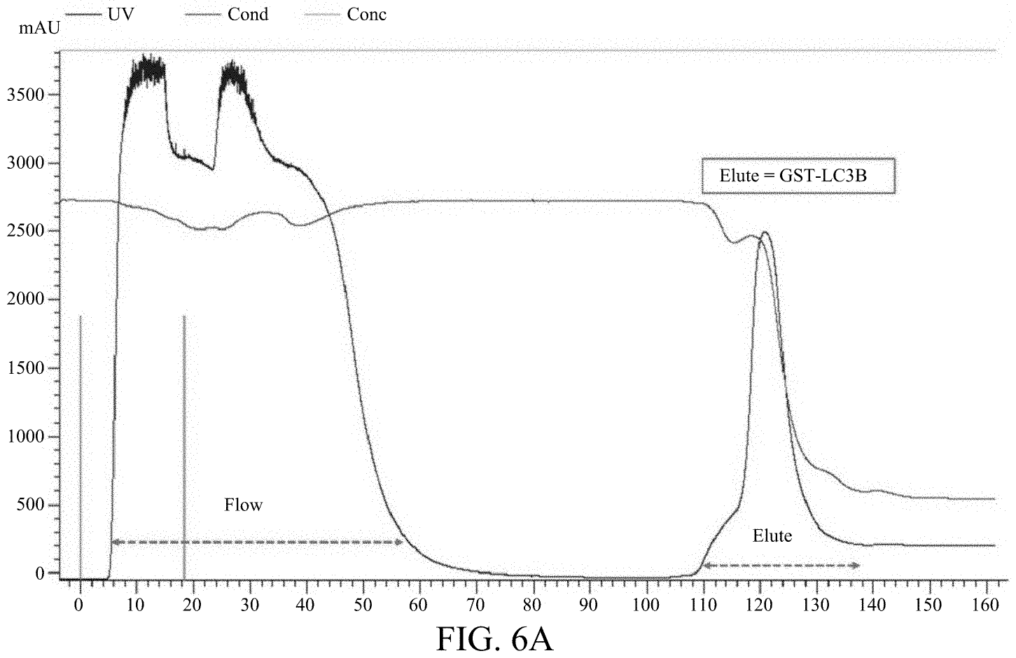

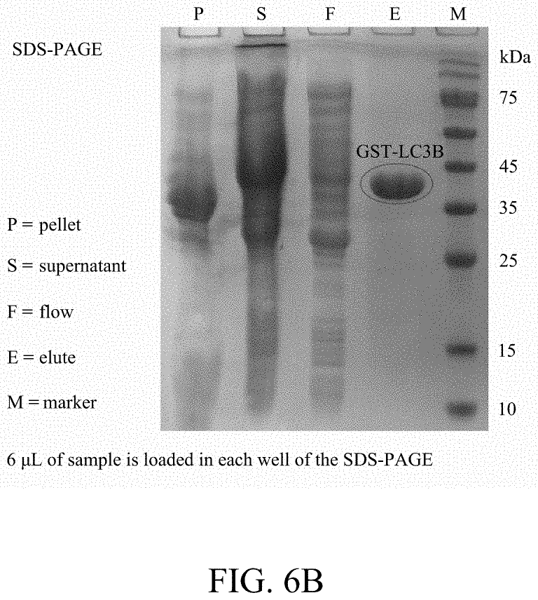

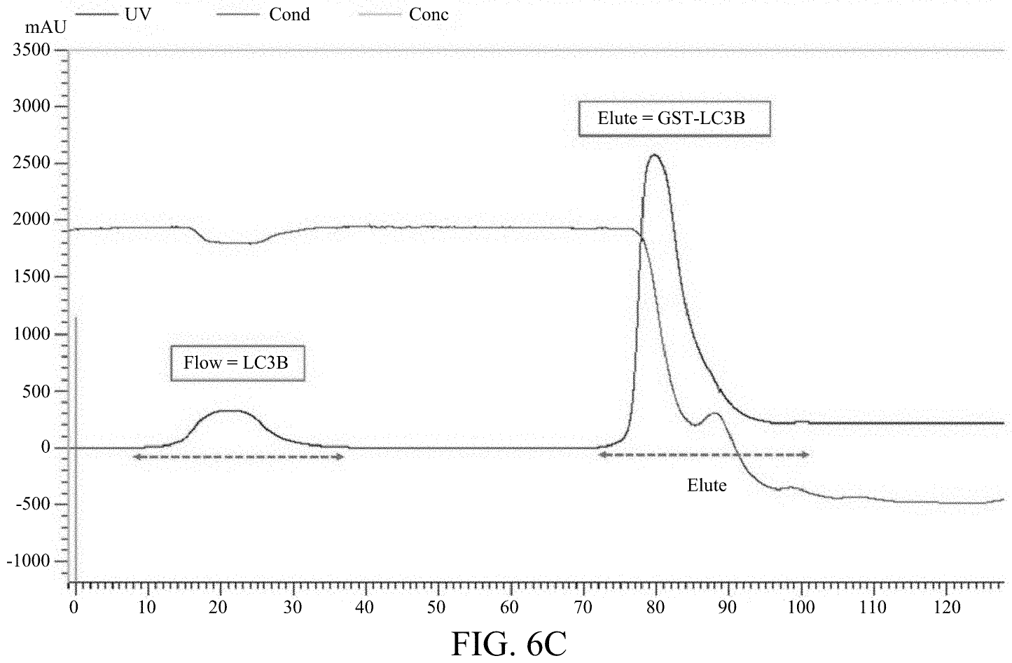

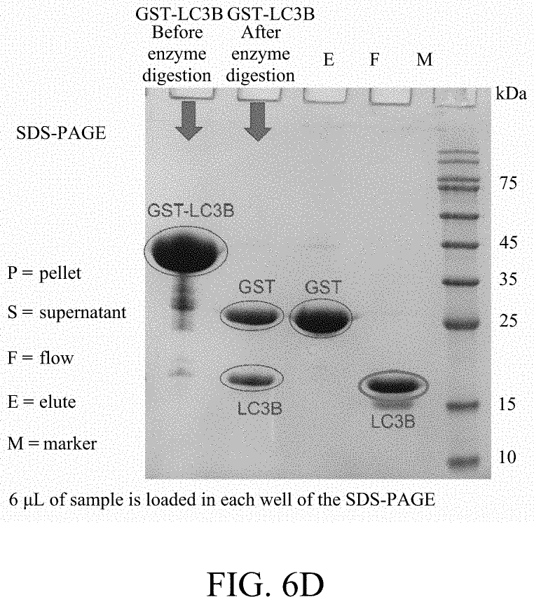

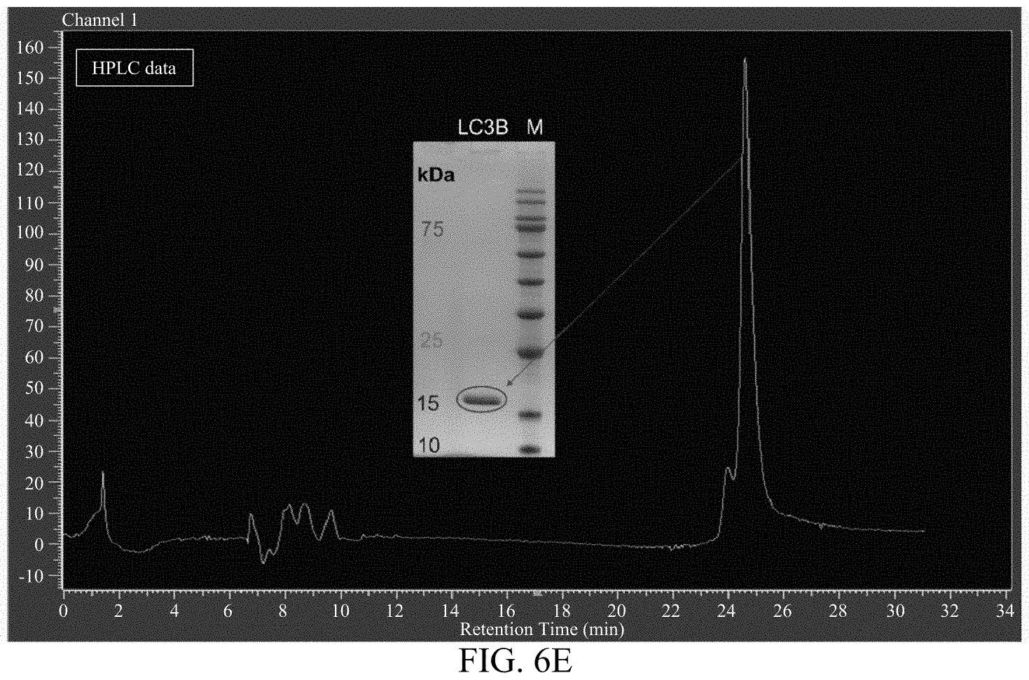

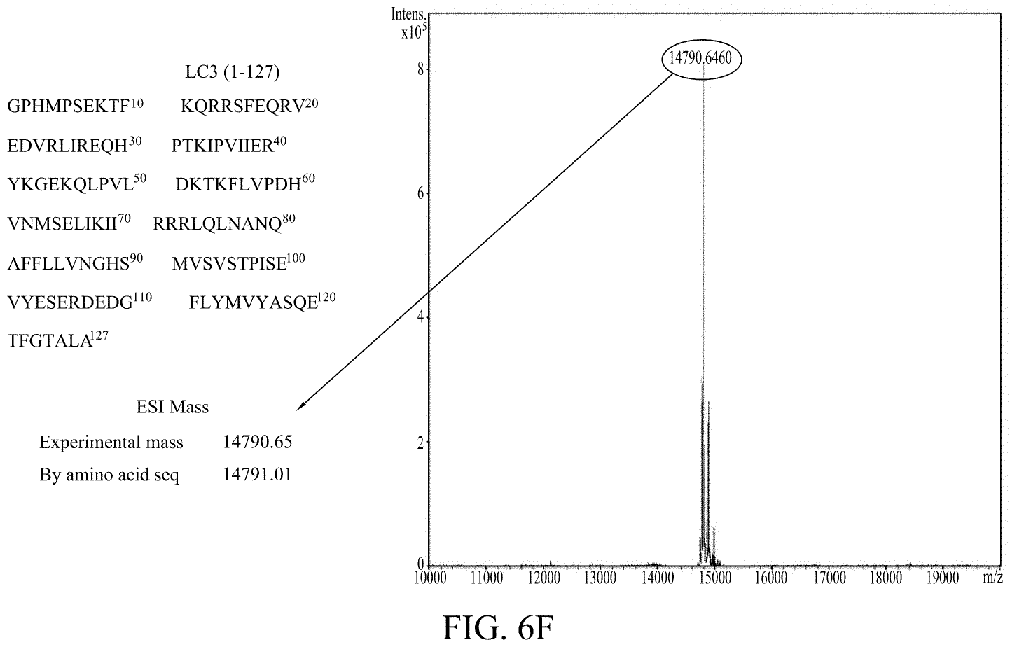

The GST-LC3B was first purified by using AKTA FPLC system with the GSTPrep FF 16/10 column (GE Healthcare Life Sciences) and elution buffer B containing 50 mM Tris-HCl and 10 mM reduced glutathione at pH 7.3. The elute containing GST-LC3B (FIG. 6A) was then buffer exchanged with buffer A and concentrated by the Amicon Ultra-15 10K device. The 2 mL of concentrated GST-LC3B was then digested with the 20 μL PreScisson protease at 4° C. for 5 hours. The cleaved GST and LC3B were then separated through the GST column. The LC3B was collected in the flow and the GST in the elute (FIG. 6C). The separation of GST and LC3B was verified by SDS-PAGE (FIG. 6D). The LC3B was further purified using high-performance liquid chromatography (HPLC). The LC3B solvated in acetonitrile, and water was eluted at 25 minutes. The purified LC3B was confirmed by SDS-PAGE (FIG. 6E) and electrospray ionization mass spectrometry (ESI-MS) (FIG. 6F). A final solution of 0.5 mM LC3B and 10% D20 was prepared for the drug titration experiment with 2D NMR HSQC.

### In Vitro Cleavage Assay of ATG4B Catalytic Activity

To assay the ATG4B catalytic activity and the inhibition effect of vinorelbine, the full-length LC3B was cloned and purified with C-Myc appended on the C-terminus and S-tag on the N-terminus. To perform the cleavage assay, 1 nM ATG4B, 250 nM C-Myc-LC3B-S-tag, and 10 μM of vinorelbine were added in the buffer composed of 150 mM NaCl, 50 mM Tris, 1 mM DTT (pH 8.0) to a final volume of 100 μL. The reagents were incubated at 37° C. for 30 minutes and then subjected to western blotting. The intensity of the band in the gel was calculated by using Image Studio Lite from LI-COR Biosciences or ImageJ.

### NMR Spectroscopy and Chemical Shift Perturbation

The ¹H-¹⁵N HSQC, ¹³C-¹⁵N HNCA, and ¹³C-¹⁵N HNCOCA spectra were collected using the triple-resonance cryogenic probe on Varian 700 MHz NMR spectrometer at 25° C. All the acquired spectra were initially processed using the VnmrJ Varian software (version 2.3), and the resulting FID file was converted to the UCSF file for the Sparky software (version 3.13) that was used to analyze the collected spectra. The resonance assignments of the ¹⁵N-labelled LC3B were done by overlapping the 2D HSQC spectra to that of BMRB 26881. Missing residues were manually assigned by two 3D experiments (HNCA and HNCOCA). To identify the vinorelbine binding interface on LC3B, the ¹H-¹⁵N HSQC spectra of the ¹⁵N-labelled LC3B and vinorelbine titration at a molar ratio of 1:1 were superimposed. The chemical shift perturbation sites were identified by displaced cross-peaks on the spectra.

### Cell Viability Assay

The cell viability was measured using the MTT assay or CellTiter-Glo Luminescent Cell Viability Assay. For MTT assay, mouse AK4.4 (1,000 cells/well) and human AsPC-1 (1,500 cells/well) cells were seeded in 96-well plates and cultured in serum-free medium (SFM) containing indicated concentrations of drugs for 48 hours. To measure the cell viability, 15 μL MTT (3-(4,5-dimethylthiazol-2-yl)-2,5-diphenyltetrazolium bromide, 5 mg/mL in PBS) was added in each well and kept at 37° C. for three hours. The precipitated crystals were then dissolved in 50 μL DMSO, and the absorbance at 570 nm was measured by Multiskan GO Microplate Spectrophotometer (Thermo Scientific, USA) to estimate the cell survival rate. To quantify the drug combination effect, the combination index was calculated when the cell viability was reduced by 50% using the following Equation (1):

$${CI}_{50} = \frac{{C}_{A , 50}}{{IC}_{50 , A}} + \frac{{C}_{B , 50}}{{IC}_{50 , B}} \qquad ( 1 )$$

where IC_{50,A} and IC_{50,B} were the inhibition concentrations of drugs A and B when the cells remained 50% of the viability; C_{A,50} and C_{B,50} were the concentrations of drugs A and B when treated in combination and resulting in a 50% reduction of the cell viability. The CI₅₀ near unity indicates an additive effect, CI₅₀<1 suggests a synergistic effect and CI₅₀>1 represents an antagonism effect.

### Western Blotting

Cells were seeded in a 12-well plate (10⁵ cells/well) and cultured in serum-free medium (SFM) with different concentrations of drugs for six hours. The plate was then washed with phosphate-buffered saline (PBS), and cells were lysed with 100 μL radioimmunoprecipitation assay buffer (RIPA buffer) for each well. The cell extracts were mixed with 4×dye (900 μL of 4×Laemmli sample buffer+100 μL of 2-mercaptoethanol) at 95° C. for ten minutes and separated by sodium dodecyl sulfate-polyacrylamide gel electrophoresis (SDS-PAGE). The fractions were then transferred to a polyvinylidene fluoride (PVDF) membrane and immersed in 5% nonfat milk at room temperature for one hour. Afterward, the transfer membrane was incubated with protein-specific primary antibodies at 4° C. overnight, washed by PBST (0.1% Tween 20 in PBS), and then incubated with horseradish peroxidase (HRP)-conjugated secondary antibodies at room temperature for one hour. The blots washed by PBST were then detected by enhanced chemiluminescence (ECL) for the quantification of proteins.

### In Vivo Treatment of Mouse Tumor Xenograft

The mouse models of pancreatic ductal adenocarcinoma (PDAC) were established by in situ implanting the AK4.4 cells (10⁵ per mouse) mixed with Matrigel into five to six-week-old FVB/NJNarl mice provided by National Laboratory Animal Center (NLAC), NARLabs, Taiwan. The drugs with different doses (10 mg/kg and 20 mg/kg) were administered via oral or intraperitoneal (IP) injection into the xenografted mice for six times at indicated days after the implantation. The mice were sacrificed 2 days after the last administration (Day 16). Proteins in tumor tissues were quantified by western blotting.

### Ensemble Structures of LC3B and Conformation Clustering

To create an ensemble structure of LC3B, both the homologous crystal structures and sampled structures using MD simulations were collected. For the homologous crystal structures, the amino acid sequence of LC3B (residue IDs: 5-120) in the crystal structure, PDB code: 2Z0DB, was used to search for the homologous sequences using SWISS-MODEL web server. Among the 1,428 structures found by SWISS-MODEL, 56 structures were retained that shared >95% sequence similarity with the query sequence. The redundant homologous structures were removed that include the ones exactly come from the query sequence and the duplicated ones presented in different biological assemblies or chains (the structure from the biological assembly 1 and chain A for each found PDB ID were chosen). Finally, the structures of which the alignment of the sequence covering the original query sequence of 116 residues were kept, which resulted in seven homologous structures.

To sample alternative conformations of LC3B, MD simulation run for 100 ns using the crystal structure from PDB code, 2Z0DB (residue IDs: 5-120), as the initial conformation. The preparation of simulation system and the running of simulation were performed using AMBER20. The protein was parameterized by ff14SB force field. The protonation states of ionizable residues were adjusted based on the pKa values predicted under pH=7 by PDB2PQR server. The protein was solvated in TIP3P water model, with 10 Å of water layer on each side of the water box. Sodium and chloride ions were added to neutralize the system and reach a final concentration of 100 mM. The simulation protocol was similar as that for protein-drug simulation described above. For the 100 ns production run, the snapshots were sampled every 20 ps, which resulted in 5,000 snapshots in total.

To summarize the ensemble structures using few representative conformations, the seven homologous structures and 5,000 sampled conformations were iteratively superposed to their mean structure in each of the iteration until the root-mean-square deviation (RMSD) of the mean structures between consecutive iteration was <10⁻⁶ Å. From the superposed structures, a 3n by 3n covariance matrix was constructed for the 3n Cartesian elements of the n superposed heavy atoms:

C_{3n×3n}=QQ^{T}

where

$${Q}_{3 n \times m} = {\frac{1}{\sqrt{m - 1}} [ {q}_{1}^{⇀} - 〈 {q}^{⇀} 〉 , {q}_{2}^{⇀} - 〈 {q}^{⇀} 〉 , … , {q}_{m}^{⇀} - 〈 {q}^{⇀} 〉 ]}_{m \times 3 n}^{T} ,$$

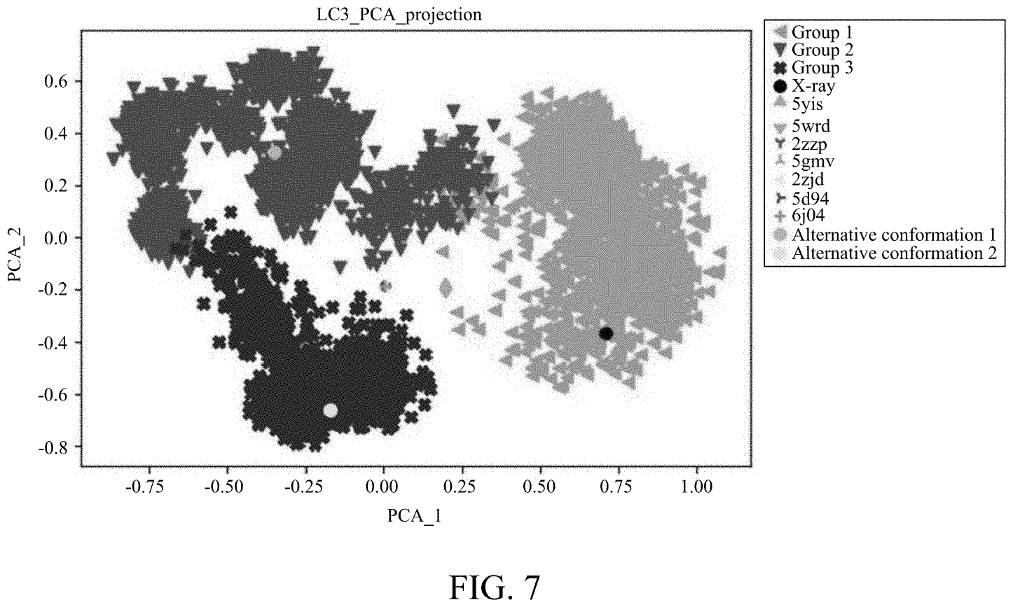

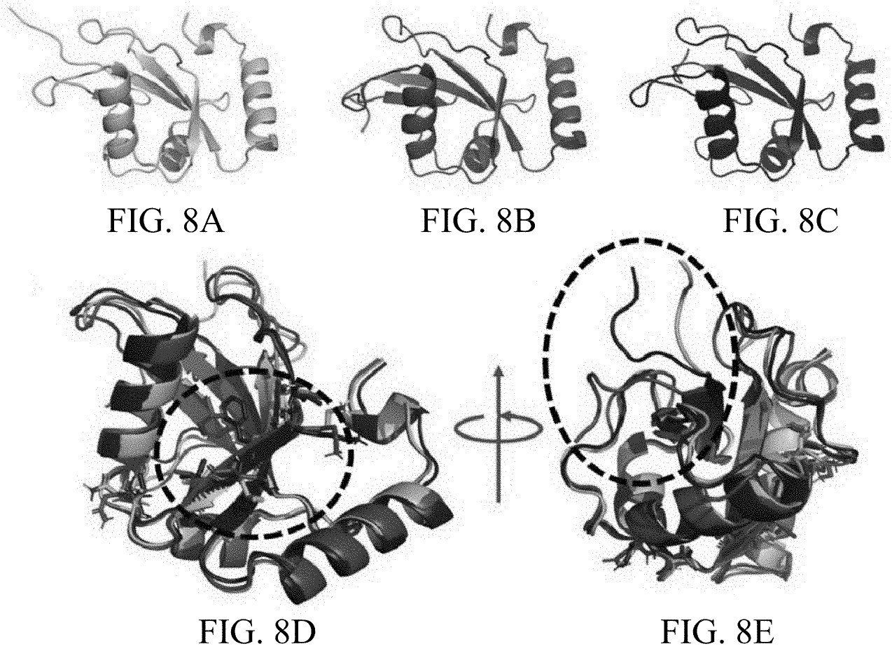

m is the number of conformations in the ensemble structures, \vec{qₗ} is the Cartesian coordinates for conformation i, [symbol] \vec{q} [symbol] is the average of the Cartesian coordinates among m conformations. Eigenvalue decomposition of the covariance matrix projected all the ensemble structures to the first two principal components (FIG. 7), where the projected points formed roughly three clusters. The conformation was clustered using hierarchical clustering implemented in SciPy, with the heavy atoms RMSD between the conformations as the distance measure and a cutoff of 2.7 Å to determine the three different clusters. For the cluster that included the initial conformation used in the simulation, the initial conformation was used as the representative structure. For the other two clusters, the structure that was closest to the mean structure in each cluster was taken as the representative structure (alternative conformation I and II) (FIGS. 8A to 8E).

### Ensemble Docking and Drug Screening Repurposed Drugs as ATG4B Allosteric Modulators

To identify potential ATG4B allosteric modulators, ensemble docking was performed by docking 2016 FDA-approved drugs on the three representative alternative conformations from the ensemble structures. All the hydrogens were added with the protonation state respecting to the pKa values under pH=7 by PDB2PQR server. All the PDBQT files for the protein receptors and drugs required for docking were generated using AutoDock Tools. AutoDock Vina was used to perform the docking experiment. Global docking was performed by setting the docking box covering all the protein structure, with a 10 Å margin on each side of the box. The exhaustiveness was set to 50. AutoDock Vina allowed at most 20 poses to be reported for each docked drug.

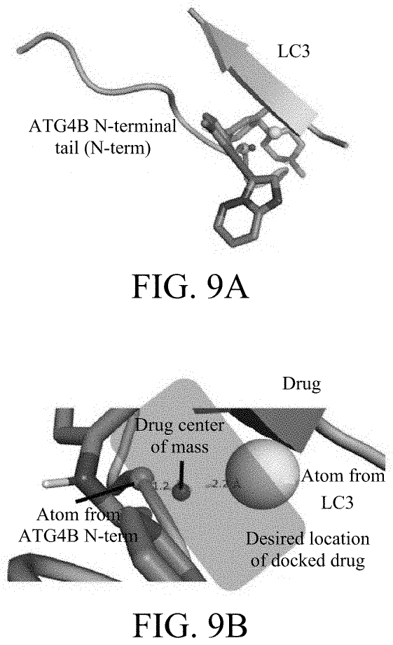

To rank the drugs from the docking results, two ranking methods were applied, i.e., normalized ranking and logarithm of odds (LOD) scoring. In normalized ranking, the docking poses with high docking affinity (≤−7 kcal/mol) were first retained. Then, three features were used to rank the drugs, including docking affinity (kcal/mol), number of contacts, and the extent of the pose located in the allosteric site. The number of contacts was defined as the number of heavy atoms that were within 4 Å between the docked drug and the residues (51-54 and 58-59) of the LC3B, which were used to interact with the ATG4B N-terminal tail (FIG. 9A). The extent of the pose located in the allosteric site was evaluated as the sum of the shortest distances of drug center of mass to any heavy atom of the residues (51-54 and 58-59) of LC3B and to any heavy atom of the residues (5-14) of ATG4B N-terminal tail that interacted with the LC3B for the intermolecular allosteric regulation (FIG. 9B). For each feature, each pose of all the sampled poses from 2016 drugs was assigned a rank according to the feature value among all sampled poses. For docking affinity, a smaller value received a higher rank (one as the highest rank). For number of contacts, a larger value received a higher rank. For the sum of the shortest distances (the extent of the pose located in the allosteric site), a smaller value received a higher rank. The rank values minus one were then normalized to between zero and one by the total number of sampled poses from 2016 drugs. Then, the normalized rank value was subtracted from one such that a pose with a best feature value resulted in a top score of one. The scores from three features were then combined (top pose score, TP score), and three was the highest score that a pose could get. The above description can be summarized as the following equation:

$$TP Score = {\sum }_{y} [ 1 - \frac{( {R}_{y} - 1 )}{n} ]$$

where y represented one of the three features, R_{y} represented the ranking of feature y among the sampled docking poses for a drug, and n was the total number of sampled docking poses of a drug. The pose with the highest TP score was specified as the representative pose of a drug among the sampled docking poses for that drug. Then, the 2016 drugs were ranked based on a drug score (D score) calculated by the three features of the representative poses with a similar manner:

$$D Score = {\sum }_{z} [ 1 - \frac{( {R}_{z} - 1 )}{m} ]$$

where z represented one of the three features, R_{z} represented the ranking of feature z among the 2016 drugs, and m (=2016) was the total number of drugs.

In the logarithm of odds (LOD) scoring, three features to rank the drugs were used, i.e., docking affinity (kcal/mol), the distance to the drug target site, and the size of poses cluster. For the distance to the drug target site, it was taken the sum of distances of drug center of mass to LC3B (residue IDs: 51-54 and 58-59) and ATG4B N-terminal tail (residues IDs: 5-14), which interacted with the LC3B for the intermolecular allosteric regulation. For the size of poses cluster, the docked poses for each drug were clustered if any pair of heavy atoms between two poses was nearby each other (<1 Å) or can be connected through intermediate poses that were nearby each other. The number of top-ranked poses (within top 10) was then counted as the size of the cluster. For each pose from all the sampled docked poses of 2016 drugs, a score was given by the sum of the logarithm of odds score from the three features:

$$LOD Score of pose x = {\sum }_{f} log \frac{{P}_{f}^{T} ( f ( x ) )}{{P}_{f}^{F} ( f ( x ) )}$$

where x represented a sampled docked pose, and f represented the feature generation function that took an input pose x and returned the corresponding feature value. f∈{the docking affinity, the distance to the drug target site, the size of poses cluster}. P_{f}^{T} was the value distribution of feature f for a docked pose sampled from a true binder. P_{f}^{F} was the value distribution of feature f for a docked pose sampled from a decoy. Thus, the LOD score represented how more likely a sampled pose was resulted from a true binder or decoy. The distribution of P_{f}^{T} and P_{f}^{F} was derived from the docking results of the 2016 drugs on 16 selected crystallized complex structures composing of proteins as drug targets and FDA-approved drugs that was also included in the 2016 drugs according to Westbrook et al., 2019 (Table 3). The 2016 drugs including the one originally in the complex were then docked to the proteins where the co-crystallized drugs had been removed. The docked pose of the co-crystallized drug that was most similar to the one in the complex structure was defined as true binder. The other docked poses and other docked poses from other 2015 drugs were treated as decoys. The feature values from these sampled docked poses were used to build the distribution of P_{f}^{T} and P_{f}^{F}. The score for each drug was assigned by the highest LOD score among the sampled docking poses and was used for ranking the 2016 drugs.

TABLE 3
The 16 protein target structures used to construct the feature distributions
of docking poses resulted from the true binders or decoys

| | | | Drug | | | | |
|---|---|---|---|---|---|---|---|
| | Chemical | Chain | Residue | Target | Drug | UniProtKB | Binding |
| PDB ID | ID¹ | ID² | ID³ | Name | Name | ID | Affinity⁴ |
| 3g0b | T22 | A | 800 | Dipeptidyl | Alogliptin | P27487 | IC₅₀: 7 nM* |
| | | | | peptidase 4 | | | |
| 4mxo | DB8 | A | 601 | Proto-oncogene | Bosutinib | P12931 | K_{d}: 0.73 nM* |
| | | | | tyrosine-protein | | | |
| | | | | kinase Src | | | |
| 4mkc | 4MK | A | 1503 | ALK tyrosine | Ceritinib | Q9UM73 | IC₅₀: 0.2 nM^{$} |
| | | | | kinase receptor | | | |
| 5p9i | 1E8 | A | 701 | Tyrosine-protein | Ibrutinib | Q06187 | — |
| | | | | kinase BTK | | | |
| 3wzd | LEV | A | 1201 | Vascular | Lenvatinib | P35968 | K_{d}: 2.1 nM* |
| | | | | endothelial | | | |
| | | | | growth factor | | | |
| | | | | receptor 2 | | | |
| 2rgu | 356 | A | 901 | Dipeptidyl | Linagliptin | P27487 | IC₅₀: 0.1 nM^{$} |
| | | | | peptidase 4 | | | |
| 2p16 | GG2 | A | 298 | Coagulation | Apixaban | P00742 | Kᵢ: 0.08 nM* |
| | | | | factor X | | | |
| 4ag8 | AXI | A | 2000 | Vascular | Axitinib | P35968 | Kᵢ: 1.1 nM* |
| | | | | endothelial | | | |
| | | | | growth factor | | | |
| | | | | receptor 2 | | | |
| 4xi3 | 29S | A | 601 | Estrogen | Bazedoxifene | P03372 | IC₅₀: 0.6 nM^{$} |
| | | | | receptor | acetate | | |
| 5csw | P06 | A | 801 | Serine/threonine- | Dabrafenib | P15056 | IC₅₀: 0.4 nM^{$} |
| | | | | protein kinase | | | |
| | | | | B-raf | | | |
| 4tvj | 09L | A | 601 | Poly [ADP- | Olaparib | Q9UGN5 | K_{d}: 0.28 nM^{$} |
| | | | | ribose] | | | |
| | | | | polymerase 2 | | | |
| 2w26 | RIV | A | 1001 | Coagulation | Rivaroxaban | P00742 | Kᵢ: 0.4 nM* |
| | | | | factor X | | | |
| 4rzv | 032 | A | 801 | Serine/threonine- | Vemurafenib | P15056 | IC₅₀: 4 nM^{$} |
| | | | | protein kinase | | | |
| | | | | B-raf | | | |
| 31xk | MI1 | A | 1125 | Tyrosine-protein | Tofacitinib | P52333 | Kᵢ: 0.2 nM* |
| | | | | kinase JAK3 | | | |
| 5ds3 | 09L | A | 1102 | Poly [ADP- | Olaparib | P09874 | IC₅₀: 0.9 nM^{$} |
| | | | | ribose] | | | |
| | | | | polymerase 1 | | | |
| 2euf | LQQ | B | 401 | Cyclin homolog | Palbociclib | Q01043 | IC₅₀: 3.8 nM^{$} |

¹The chemical ID for the access of the summary page of the drug in Protein Data Bank.
²The protein chain ID in the PDB entry used in this study.
³The residue ID of the drug in the selected chain ID.
⁴The annotated experimental binding affinity associated with the PDB entry from Binding MOAD (*), BindingDB ($), or PDBBind (#).

The two ranking methods described above were applied to the docking results performed on each of the three alternative conformations, which resulted in six sets of ranked drug lists (Table 4). The top-ranked drugs were chosen for testing their effects on the inhibition of cancer cell viability from the following rules:

Choice 1: the top three-ranked drugs in each of the six ranked drug lists, including ponatinib, suramin, ergotamine, dolutegravir, conivaptan, moxidectin, nilotinib, ledipasvir, paritaprevir, aclacinomycin A, ethynyl estradiol, closantel, saquinavir, dihydroergocristine, itraconazole, daclatasvir, (+)-butaclamol, and temsirolimus;

Choice 2: the drugs ranked within top 20 in four out of the six ranked drug lists, including suramin, ergotamine, nilotinib, ledipasvir, itraconazole, and Evans blue;

Choice 3: the drug ranked within top five in both the ranked drug lists resulted from the docking results using the conformation in the crystal structure, gliquidone;

Choice 4: the drug ranked within top 20 by either of the two ranking methods from both the docking results using the conformation in the crystal structure and alternative conformation I, CP-640186;

Choice 5: the drug with most docked poses of which the docking affinity were <−9 kcal/mol among the 2016 drugs when docked on alternative conformation II.

Table 4 below illustrates the top 20 drugs in each of the six ranked drug lists resulted from three conformations of LC3B and two ranking methods. The drugs chosen for further assay of inhibition on cancer cells viability by Choice 1 were indicated by the symbol “@”; the drugs chosen by Choice 2 were marked as “#”; the drugs chosen by Choice 3 were shown in the symbol of “+”; the drugs chosen by Choice 4 were indicated by the symbol “$”, and that chosen by Choice 5 was shown in the symbol “&”. The drugs showed >50% inhibition of the viability on HCT116 cells were highlighted in italic. The drugs showed >10% differences in inhibition of cell viability between shATG4B/HCT116 and shCtrl/HCT116 were highlighted in bold.

TABLE 4

| | X-ray | | Alternative | | Alternative | |
|---|---|---|---|---|---|---|
| | (2Z0DB) | | conformation 1 | | conformation 2 | |
| LC3 | Normal | LOD | Normal | LOD | Normal | LOD |
| Ranking | rank | rank | rank | rank | rank | rank |
| 1 | Ponatinib^{@} | Suramin^{@#} | Ergotamine | Dolutegravir^{@} | Conivaptan^{@} | Moxidectin^{@} |
| 2 | Nilotinib^{@#} | Ledipasvir^{@#} | Paritaprevir^{@} | [symbol] | [symbol] | Closantel |
| | | | | [symbol] ^{@} | [symbol] ^{@} | |
| 3 | Saquinavir^{@} | Dihydroergocristine | Itraconazole^{@#} | Daclatasvir^{@} | (X) (+)- | Temsirolimus^{@} |
| | | mesylate^{@} | | | Butaclamol- | |
| | | | | | HCl | |
| 4 | Chlorhexidine | Gliquidone⁺ | Fluorescein | Hesperidin | Venetoclax | Nystatin |
| 5 | Gliquidone⁺ | Paritaprevir | (R)-(−)- | Ponatinib | Estradiol | Evans Blue^{#} |
| | | | Apomorphine | | | |
| 6 | Tucidinostat | Micafungin | Posaconazole | Elbasvir | Suramin^{#} | Ziprasidone |
| 7 | Pranlukast | Evans Blue^{#} | Azilsartan | Itraconazole^{#} | Tolvaptan | Temoporfin |
| 8 | Montelukast | Aclacinomycin | Ellagic acid | Ergotamine | Nilotinib^{#} | Vancomycin |
| | | A | | | | |
| 9 | Evans Blue^{#} | Valrubicin | Entrectinib | Evans Blue^{#} | Plicamycin | Elbasvir |
| 10 | Ledipasvir^{#} | Menaquinone-4 | Olopatadine | Ziprasidone | Temoporfin | Everolimus |
| 11 | Itraconazole^{#} | Eltrombopag | Droperidol | Suramin^{#} | Glycyrrhizic | Norvancomycin |
| | | | | | acid | |
| 12 | Lumacaftor | Tucidinostat | Lasofoxifene | CP-640186 | (+)- | Eltrombopag |
| | | | | hydrochloride^{$} | Tubocuraraine | |
| | | | | | chloride | |
| 13 | Lanatoside C | Chlorhexidine | Suramin^{#} | Nilotinib^{#} | Cyproheptadine | Novobiocin |
| 14 | Ergotamine | Doxorubicin | Rilpivirine | Ledipasvir^{#} | Itraconazole^{#} | Levocabastine |
| 15 | Menaquinone-4 | Pirarubicin | Nilotinib^{#} | Bicalutamide | Ledipasvir^{#} | Dutasteride |
| 16 | Siponimod | Atovaquone | Dutasteride | Cabozantinib | Estrone | Montelukast |
| 17 | Dihydroergocristine | Dihydroergotamine | Telotristatethyl | Temsirolimus | Leuprolide | Lifitegrast |
| | | | | | Acetate | |
| 18 | Fertirelin | Telmisartan | Talazoparib | Paritaprevir | Ergotamine | Micafungin |
| 19 | CP-640186 | Mozavaptan | Adapalene | Risperidone | Rolapitant | Lanatoside C |
| | hydrochloride^{$} | | | | | |
| 20 | Pazopanib | Bromocriptine | Eltrombopag | Larotrectinib | Netupitant^{&} | Entrectinib |

### In Silico Peptide Design

To further improve the peptides for the allosteric inhibition of ATG4B, in silico methods were used including Docking and MD simulations for the designs of new peptides. First, the ATG4B N-terminal tail was retrieved from the crystal structure (residues 5-18, SEQ ID NO: 18 (TLTYDTLRFAEF), N-term-12), and the first three residues (TLT) from the N-terminal and four residues (RFAE) in the middle of the peptide were removed due to their flexibilities and fewer contacts to LC3B observed from the crystal structure and MD simulations (Table 5). For example, Table 5 below illustrates the contact number and RMSF of residues of N-term-12 to LC3B in the crystal structure and a 30 ns MD simulation. The crystal structures of LC3B and N-term-12 were derived from PDB code: 2Z0D. N-term-12 refers to “SEQ ID NO: 18 (TLTYDTLRFAEF)” (residue IDs: 5-16).

TABLE 5

| | | | Average | |
|---|---|---|---|---|
| | | | Contact Number | |
| | | Average | Difference² | |
| | Contact | Contact | over 30 ns MD | |
| | Number¹ | Number¹ | Simulations | |
| | in Crystal | over 30 ns MD | to Crystal | RMSF |
| Residue | Structure | Simulation | Structure | (Å)³ |
| T5 | 0 | 0.66 | 0.66 | 4.57 |
| L6 | 4 | 1.79 | −2.22 | 2.75 |
| T7 | 2 | 2.42 | 0.42 | 1.11 |
| Y8 | 9 | 8.34 | −0.66 | 0.70 |
| D9 | 8 | 7.34 | −0.66 | 0.70 |
| T10 | 5 | 3.23 | −1.77 | 0.75 |
| L11 | 6 | 5.45 | −0.55 | 0.75 |
| R12 | 0 | 0.06 | 0.06 | 1.17 |
| F13 | 2 | 0.69 | −1.31 | 1.46 |
| A14 | 0 | 0.01 | 0.01 | 1.56 |
| E15 | 5 | 4.96 | −0.04 | 1.13 |
| F16 | 3 | 4.44 | 1.44 | 1.42 |

¹The contact number was defined as the number of heavy atoms in the residue that were within 4 Å to LC3B.
²The contact number difference was defined as the difference of the contact number between each snapshot and the crystal structure.
³RMSF: the root-mean-square fluctuation of the Cα atom in the 30 ns MD simulations.

To replace the removed four residues (RFAE) and link the remaining segments, linkers of one, two, or three glycines (G) were considered. To determine the suitable length of the linker, AutoDock Vina was used with default parameters to dock the peptides connected by these three linkers into LC3B. The number of native contacts of N-term-12 was defined as the number of heavy atoms on LC3B that were within 4 Å of the fourth to seventh (YDTL) and last (F) residues of N-term-12. Then, the remained native contacts (normalized by the number of native contacts to give a ratio between zero and one) were calculated for each pose of the docked peptide using the same set of residues (YDTL and E). The results indicated that the one with two glycines as the linker (SEQ ID NO: 14 (YDTLGGF)) had pose retaining the highest native contacts as N-term-12 (Table 6), and the length of the GG linker was the shortest one that can accommodate the gap (8.14 Å) between residues of L and F after removing the middle residues, RFAE, from N-term-12. Specifically, Table 6 illustrates the native contact ratio of docked peptides containing different lengths of glycine linker. The number of native contacts was defined as the number of heavy atoms in LC3B that were within 4 Å of N-term-12 in the crystal structure.

TABLE 6

| SEQ ID NO: 21 | | | SEQ ID NO: 14 | | | SEQ ID NO: 22 | | |
|---|---|---|---|---|---|---|---|---|
| (YDTLGF) | | | (YDTLGGF) | | | (YDTLGGGF) | | |
| | | Native | | | Native | | | Native |
| Pose | Affinity¹ | Contact | Pose | Affinity¹ | Contact | Pose | Affinity¹ | Contact |
| ID | (kcal/mol) | Ratio² | ID | (kcal/mol) | Ratio² | ID | (kcal/mol) | Ratio² |
| 1 | −6.8 | 0 | 1 | −7 | 0 | 1 | −7.7 | 0 |
| 2 | −6.8 | 0 | 2 | −7 | 0 | 2 | −7.7 | 0 |
| 3 | −6.8 | 0 | 3 | −6.8 | 0.84 | 3 | −7.5 | 0 |
| 4 | −6.7 | 0 | 4 | −6.8 | 0 | 4 | −7.3 | 0 |
| 5 | −6.7 | 0 | 5 | −6.8 | 0 | 5 | −7.3 | 0 |
| 6 | −6.7 | 0 | 6 | −6.8 | 0 | 6 | −7.2 | 0 |
| 7 | −6.6 | 0 | 7 | −6.8 | 0 | 7 | −7 | 0.82 |
| 8 | −6.6 | 0.42 | 8 | −6.7 | 0.36 | 8 | −7 | 0 |
| 9 | −6.6 | 0 | 9 | −6.7 | 0 | 9 | −6.9 | 0.21 |
| 10 | −6.6 | 0 | 10 | −6.7 | 0 | 10 | −6.9 | 0 |

¹The binding affinity reported from AutoDock Vina.
²The native contact ratio was the number of retaining native contacts in the docked pose divided by the number of native contacts formed by N-term-12. The value was between zero and one.

Next, point mutation on each residue of the above peptide was performed to the other 19 residues, and 134 peptides, including SEQ ID NO: 14 (YDTLGGF) (7×19+1=134), were again subject to docking screening on LC3B's LIR binding site using AutoDock Vina. All the outputted poses were first compared to the binding mode of N-term-12 by calculating the RMSD values between the Cα atoms of the first four and last residues in the docking poses, and the fourth to seventh (YDTL) and the last residues (F) of the N-term-12. The docking poses that were similar enough to the N-term-12 (RMSD <10 Å) were retained, and then sorted based on AutoDock Vina-predicted binding affinities (Table 7). Specifically, Table 7 illustrates the results of point-mutated peptides/poses (20 docking poses for each peptide) docked to LC3B. The upper part of the table listed the top-ranked peptides poses having the smallest RMSD from the position of ATG4B's N-terminal tail (SEQ ID NO: 20 (YDTLRFAEF)) and rank-ordered by Vina predicted affinity. Those prioritized poses that belong to the “wild type” peptide, SEQ ID NO: 14 (YDTLGGF), were collected and shown separately at the bottom of the table. Peptides marked with an asterisk (*) indicated the selected peptides subject to further 100 ns MD simulations for binding stability assessment to LC3B. The top-ranked three peptides (SEQ ID NO: 1 (YDYLGGF), SEQ ID NO: 2 (YDTLGIF), SEQ ID NO: 3 (YDTLYGF), Table 7, upper, indicated by arrow) and the top-ranked pose of SEQ ID NO: 14 (YDTLGGF) (Table 7, bottom, indicated by arrow), as a control, were selected for further 100 ns simulations to assess their binding stabilities. The simulation results suggested that SEQ ID NO: 2 (YDTLGIF) was a promising candidate, compared with the other three, to form stable interactions with LC3B as the peptide is stable through the 100 ns simulation. This peptide, as an interference of the inter-molecule allosteric regulation by LC3B, is thus chosen for the ATG4B cleavage assays to assess its inhibition to ATG4B.

TABLE 7

| | Pose | | RMSD |
|---|---|---|---|
| Peptide Sequence | Rank | Affinity | Ctrl¹ |
| *SEQ ID NO: 1 | 1 | -7.8 | 8.65 |
| (YDYLGGF) | | | |
| SEQ ID NO: 1 | 2 | -7.8 | 8.82 |
| (YDYLGGF) | | | |
| *SEQ ID NO: 2 | 1 | -7.7 | 4.40 |
| (YDTLGIF) | | | |
| *SEQ ID NO: 3 | 1 | -7.7 | 8.35 |
| (YDTLYGF) | | | |
| SEQ ID NO: 4 | 2 | -7.6 | 7.36 |
| (YDTLSGF) | | | |
| SEQ ID NO: 4 | 3 | -7.6 | 5.88 |
| (YDTLSGF) | | | |
| SEQ ID NO: 1 | 6 | -7.6 | 8.09 |
| (YDYLGGF) | | | |
| SEQ ID NO: 5 | 1 | -7.6 | 8.73 |
| (YPTLGGF) | | | |
| SEQ ID NO: 6 | 1 | -7.5 | 9.52 |
| (PDTLGGF) | | | |
| SEQ ID NO: 7 | 2 | -7.5 | 7.44 |
| (YDTLGGP) | | | |
| SEQ ID NO: 8 | 1 | -7.5 | 6.43 |
| (YDTLTGF) | | | |
| SEQ ID NO: 3 | 2 | -7.5 | 6.64 |
| (YDTLYGF) | | | |
| SEQ ID NO: 9 | 1 | -7.5 | 9.01 |
| (YDTPGGF) | | | |
| SEQ ID NO: 10 | 1 | -7.5 | 8.46 |
| (YDTSGGF) | | | |
| SEQ ID NO: 11 | 2 | -7.5 | 9.53 |
| (YDWLGGF) | | | |
| SEQ ID NO: 1 | 8 | -7.5 | 9.76 |
| (YDYLGGF) | | | |
| SEQ ID NO: 1 | 9 | -7.5 | 6.15 |
| (YDYLGGF) | | | |
| SEQ ID NO: 12 | 1 | -7.5 | 4.12 |
| (YWTLGGF) | | | |
| SEQ ID NO: 13 | 3 | -7.5 | 9.45 |
| (YYTLGGF) | | | |
| SEQ ID NO: 13 | 6 | -7.5 | 9.27 |
| (YYTLGGF) | | | |
| *SEQ ID NO: 14 | 2 | -7.1 | 8.34 |
| (YDTLGGF) | | | |
| SEQ ID NO: 14 | 4 | -6.9 | 8.54 |
| (YDTLGGF) | | | |
| SEQ ID NO: 14 | 5 | -6.9 | 6.61 |
| (YDTLGGF) | | | |
| SEQ ID NO: 14 | 8 | -6.8 | 4.59 |
| (YDTLGGF) | | | |

¹RMSD Ctrl: root-mean-square deviation of peptide to ATG4B’s N-terminal tail (SEQ ID: 20 (YDTLRFAEF)) by considering the C of the first four and last residues.
*The selected peptides subject to further 100 ns MD simulations for binding stability assessment to LC3B.

### Synthesis of Peptides

All the peptides used in this study were synthesized and purchased from LifeTein LLC and Kelowna International Scientific Inc.

System preparation and defining the interface residues of 3CL^{pro} Protein targets were prepared by performing a MD simulation of 200 ns starting from the homodimer 3CL^{pro} (PDB id: 6Y2E). Consequently, the chain A was extracted from the last frame, and additional 100 ns simulation was performed. Finally, the last frame of the simulation (End of Monomer Simulation) was termed EMS. The EMS monomer served as the target conformation for screening the interface drug (by global docking using Autodock Vina).

To find drugs blocking the combination of two monomers, the interface residue of the homodimer was first defined by the contact number where a contact was the atom-atom distance small than or equal to 4 Å. The interface residues of the 3CL^{pro} homodimer were defined by the contact number of a residue larger than six. Nine interfacial residues in the domain II SER121, PRO122, SER123, GLY124, VAL125, SER139, PHE140, LEU141, and GLU166, and six residues in the N-terminus SERI, GLY2, ARG4, METE, ALAI, and PROS were found.

### The Global Docking and Drug Cluster Analysis

AutoDock Vina was used for drug screening, where each of the 2016 FDA-approved drugs was docked in the protein and sampled for 20 poses. The docking box was set to cover the whole structure of the protein target with an additional 5 Å margin patched on each side of the docking box. Exhaustiveness of 20 was set to give comprehensive screening results. The procedure was termed as the global docking. Consequently, the global docking was used on the EMS structure, and the output docking poses were submitted to further drug clustering analysis.

Considering that the contribution of conformational entropy could facilitate the binding free energy, the poses of global docking were clustered for each drug by the hierarchical clustering method. The number of poses in each cluster for each drug was calculated, and the number of poses in the largest cluster was termed as the largest cluster size (LCS). The mean LCS of all the FDA drugs was termed MLCS. The drugs whose LCS are smaller or equal to the rounded MLCS were removed. The remaining drugs were served as candidate drugs for further drug contact analysis.

### The Drug Contact Analysis

Next, interface drugs having high contact with domain II and N-terminus were chosen. A contact was formed if a heavy atom in the drug is <4 Å from a heavy atom of interface residues defined in the above section. The top 50 drugs having the highest contact number were selected for further MD simulations and MM/PB(GB)SA analysis.

### The Normal Mode-Based Time-Dependent Linear Response Theory (NMA-Td-LRT) Predicted Allosteric Sites and Intramolecular Communication Centers (ICCs)

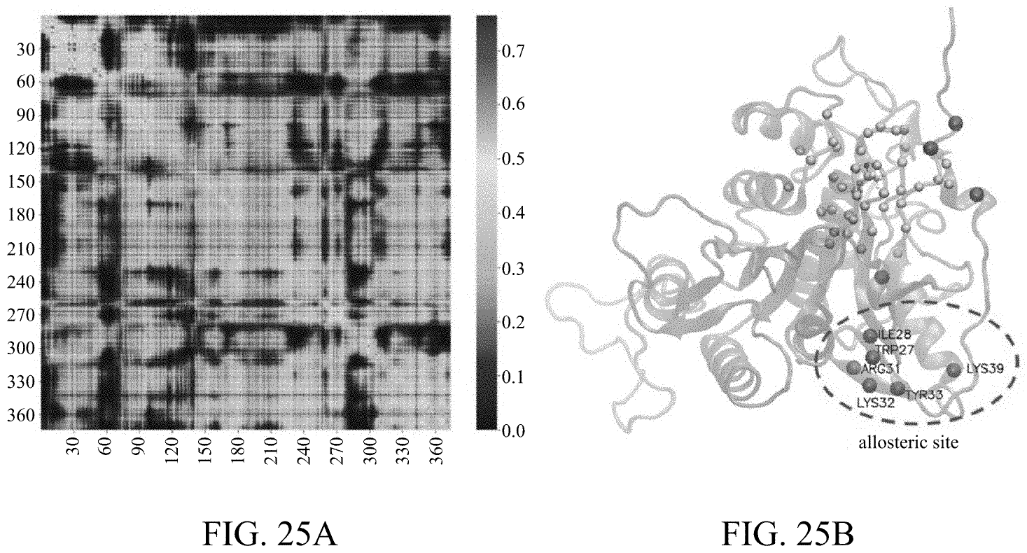

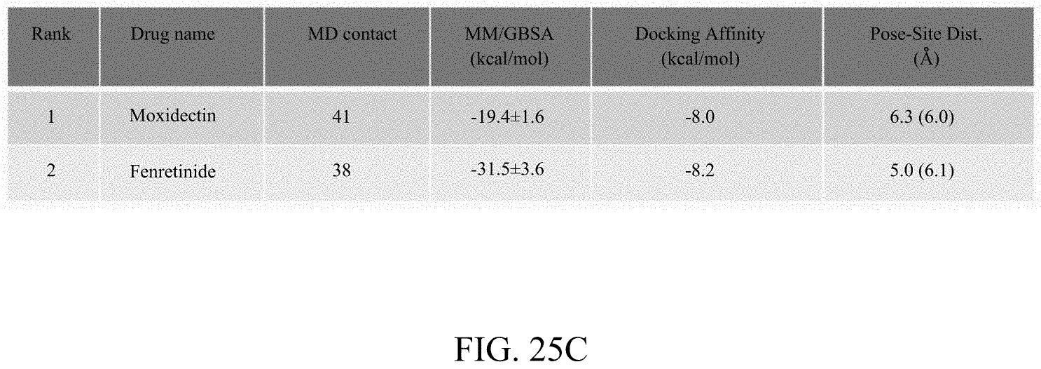

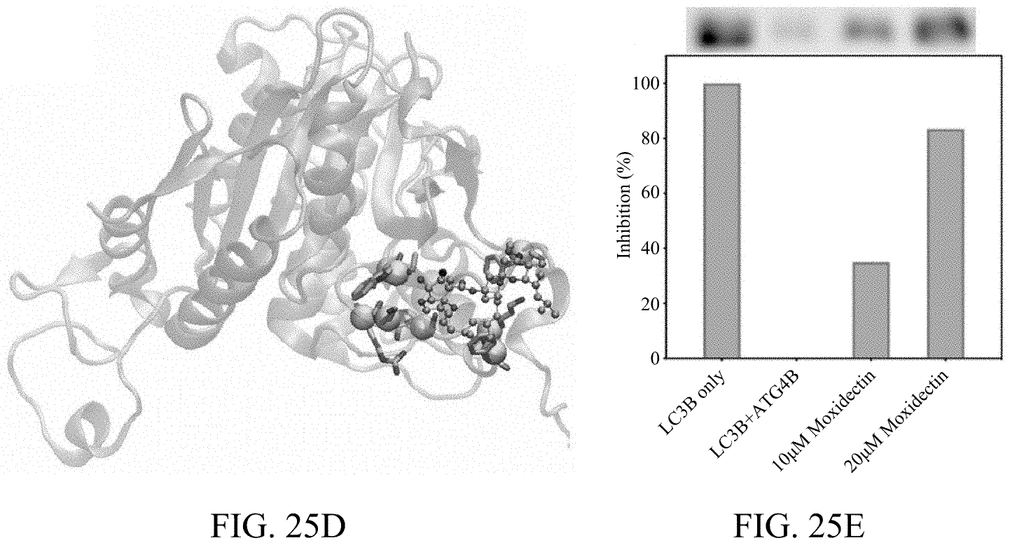

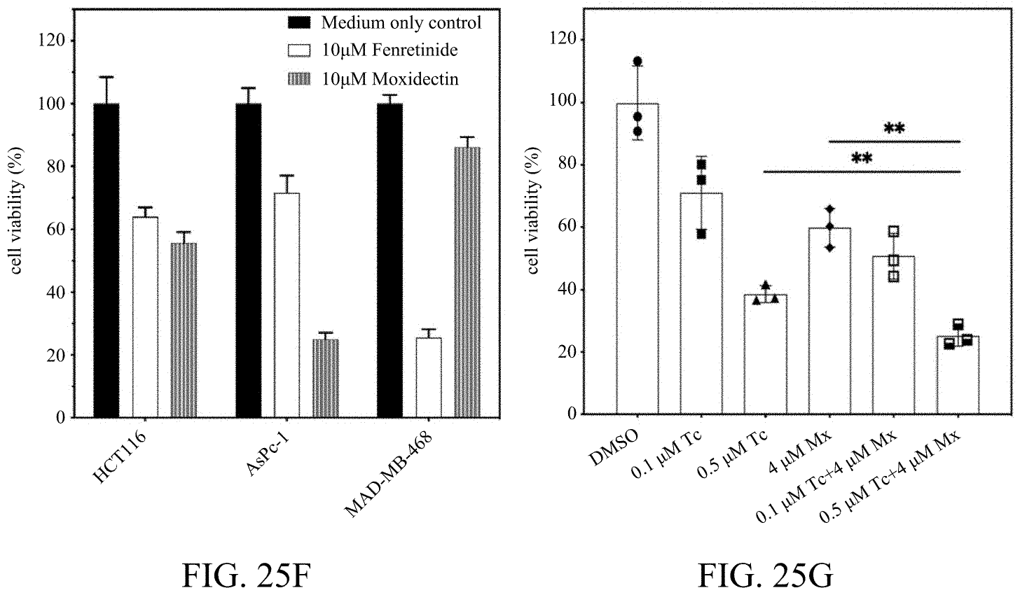

Huang's method (Huang et al., 2019, bioRxiv) is performed by the normal mode-based td-LRT for the closed form of ATG4B (PDB code: 2CY7), where perturbation forces are given to perturb each of 41 CA atoms (within 8 Å of the active site) along 133 uniformly distributed directions. Consequently, the calculated CGCM was shown in FIG. 25A. It can be seen that there was a group of “hot-spot” near the residue 30 in the CGCM referring to the ICCs. Then, the communication score was calculated, and the top-10 ICCs marked by red balls were taken to identify potential allosteric sites.

Drug Docking and MD Simulations to Repurpose FDA Approved Drugs that Target Site 1 of ATG4B

The ATG4B structure adopting an active enzyme conformation (or the “open form”; PDB ID: 2Z0D) was taken for screening of FDA drugs that bind the identified allosteric site. All the missing loops were patched, and the catalytically inert mutation, H280A, was back-mutated with the aids of SWISS-MODEL web service (Waterhouse et al., 2018, Nucleic Acids Research 46, 296-303).

Hydrogen atoms of ionizable residues in the ATG4B structure were added or removed per their protonation states, calculated by PDB2PQR web service (Dolinsky et al., 2007, Nucleic Acids Research 35, 522-525). A set of 2016 FDA-approved drugs compiled from the catalogs of MedChemExpress (MCE) FDA-Approved Drug Library (Cat. No.: HY-L022) and Screen-Well FDA Approved Drug Library (Version 1.5) of Enzo Life Sciences, Inc. were used for the drug screening. The 3D structures of drugs were built by BIOVIA Discovery Studio (Dassault Systèmes BIOVIA, Discovery Studio Modeling Environment, Release 2017, San Diego: Dassault Systèmes, 2016) with appropriate adjustment of the protonation state of all ionizable functional groups under pH=7. The docking software, AutoDock Vina (Trott O. and Olson A. J., 2010, J. Comput. Chem. 31, 455-461), was used to perform virtual drug screening. The PDBQT files required as the input files were generated using AutoDock Tools (Morris et al., 2009, J. Comput. Chem. 30, 2785-91). The search box was adjusted to cover the whole protein structure for global search, with a space of 5 Å thickness padded from each side of the box to the protein. The exhaustiveness was set to 100. For each drug, 20 docking poses were allowed to be generated by AutoDock Vina.

To analyze the docking result and choose the drug candidates for repurposing as ATG4B intramolecular allosteric drugs, in addition to the binding affinity obtained for each docking pose, the distance, as a second feature, from the pose to the target sites was calculated. Within the top 10 ICCs having the highest communication scores (CSs), those >10 Å away from the active sites and spatially clustered were selected to discover a single allosteric site comprising TRP27, TYR33, ARG31, LYS39, LYS32 and ILE28 as shown in the red spheres of FIG. 25B. The pose distance was defined as the shortest distance between the heavy-atom mass center of a drug and the closest heavy atom of an ICC residue in the allosteric site. To identify drugs with relatively high affinity in short distance from the target site, only the docking poses with affinity <−7 kcal/mol and distance <7 Å were retained. This results in 7 poses from 6 drugs. The poses were then rank-ordered in accord with affinity and distance. The poses were first ranked by affinity only (low affinity ranks higher) and distance only (the shorter, the better) before they were ranked based on the smallest sum of the ranks or the rank-converted scores described in the equation as bellow.

$$Score of a docking pose = {\sum }_{f = 1}^{2} [ 1 - \frac{( {R}_{f} - 1 )}{N} ] ,$$

where feature f represents the pose affinity or distance. R_{f} represents the rank of a pose according to the f feature (the highest rank is 1; the lowest is N). N is the total number of the retained poses, which is 7 in this case.

Protein expression and purification for the study of prediction of the allosteric sites in ATG4B Human ATG4B with N-terminal 6xHis-tag and human LC3B with both N-terminal 6xHis-tag and C-terminal S-tag were cloned into pETDuet-1 expression plasmids as previously described (Shu, C W et al., 2010, Autophagy 6, 936-947), subsequently transformed into E. coli BL21 (ECOS 21, Yeastern Biotech Co., FYE207-40VL). A single colony harboring the correctly in-frame inserted gene was inoculated into an overnight culture that was then diluted 100-fold in fresh Luria-Bertani broth with 50 μg/mL ampicillin for growing at 37° C. with 200 rpm agitation for 2 to 3 hours until the optical density of the culture at 600 nm reaches 0.4 (around 3×10⁸ cells/mL). The recombinant proteins were then induced by 0.1 mM isopropyl β-D-1-thiogalactopyranoside (IPTG) and cultivated at 20° C. for another 5 hours.

The expression cell culture was harvested by 12,000×g centrifugation at 4° C. for 20 minutes, and the cell pellets of 1 liter broth were resuspended in a 10 mL lysis buffer (50 mM Tris-HCl, pH 7.4, 300 mM NaCl, 20 mM imidazole). The 10 mL cells were lysed by sonication and centrifuged at 38,500×g for 15 minutes at 4° C., and the supernatant containing the 6xHis-tagged recombinant proteins was filtered by 0.8 μm membrane before subjected to 3 mL Ni-NTA-agarose (Qiagen, 30250) gravity column. Subsequently, the sample buffer comprising 10 mM Tris-HCl, pH 7.4, 150 mM NaCl, 10 mM β-mercaptoethanol, and 0.1% Triton X-100 was applied throughout the purification, including pre-washing by 20 mM imidazole and collection of elution with 100 mM imidazole. The ATG4B and LC3B proteins were concentrated and frozen with 20% glycerol. The protein purity was verified >90% by Coomassie blue staining on SDS-PAGE, and the quantity was measured by bicinchoninic acid assay.

### ATG4B Enzyme Activity Assay by S-Tag-Based Immunoblotting for the Study of Prediction of the Allosteric Site in ATG4B

ATG4B enzyme activity assay was analyzed by immunoblotting. The purified recombinant ATG4B (5 to 10 nM) was incubated with 500 nM C-terminal S-tagged LC3B in 100 μL reaction buffer containing 50 mM Tris-HCl, pH 8.0, 150 mM NaCl, and 1 mM dithiothreitol at 37° C. for 2 hours. Reactions were stopped by addition of 5X SDS-sample buffer and 95° C. heated for 5 minutes before loaded to 12% SDS-PAGE gel for electrophoresis. Afterwards, the samples were then transferred to nitrocellulose membranes (PALL, Biotrace NT 66485) for immunoblotting analyses. The membrane was blocked with 5% skim milk (Sigma-Aldrich, 70166) in TBST buffer (TBS with 0.05% Tween-20) for 1 hour at room temperature with mild shaking, and then incubated with anti-S-tag (Bethyl, A190-135A), anti-c-Myc (Sigma-Aldrich, C3956) or anti-ATG4B (Sigma-Aldrich, A2981) primary antibodies in TBST buffer containing 5% BSA for overnight at 4° C. with mild shaking. The proteins were then probed with peroxidase conjugated mouse anti-rabbit IgG secondary antibody (Santa Cruz, sc-2357-CM) with 5% skim milk in TBST buffer for 1 hour at room temperature. The membranes were then treated with enhanced chemiluminescence (ECL) reagent (GE Healthcare, RPN2232) for band intensity detection by the ImageQuant LAS 4000 Imaging System (Cytiva, USA). The intensities of remaining substrate LC3B after enzymatic cleavage were quantified by the software ImageJ.

### Tumor Cell Culture and Cell Viability Assay by WST-1 for the Study of Prediction of the Allosteric Site in ATG4B

Three tumor cells were tested, i.e., HCT116 (colorectal cancer), AsPC-1 (pancreatic cancer) and MDA-MB-468 cell lines (breast cancer). Human colorectal carcinoma cell line HCT116 (catalog number: BCRC 60349, Bioresource Collection and Research Center (BCRC), Hsinchu City, Taiwan) was maintained in the culture medium: McCoy's 5a medium (catalog number: 16-600-082, Gibco, Waltham, Mass.) with 1.5 mM L-glutamine (Gibco) and 10% fetal bovine serum (FBS, Gibco). Human pancreatic adenocarcinoma cell line AsPC-1 (catalog number: BCRC 60494, BCRC) was maintained in the culture medium: RPMI 1640 medium (catalog number: 11875168, Gibco) with 2 mM L-glutamine, 4.5 g/L glucose (Gibco), 10 mM HEPES (Gibco), 1 mM sodium pyruvate (Gibco) and 10% fetal bovine serum. Human breast adenocarcinoma cell line MDA-MB-468 (catalog number: ATCC HTB-132, American Type Culture Collection (ATCC), Manassas, Va.) was maintained in the culture medium: Leibovitz's L-15 medium (catalog number: 11415064, Invitrogen, Waltham, Mass.) with 2 mM L-glutamine and 10% fetal bovine serum. All cells were incubated in a humidified atmosphere without CO₂ in air at 37° C. to serve as the control for the following assays.

For cell viability assay, tumor cells (1×10⁵/mL) were suspended in the medium without testing drugs and then inoculated in 96-well plates (100 μL per well) for attachment first. After a 12-hour incubation to allow for cell attachment, the conditioned medium was withdrawn. Then, fenretinide and moxidectin (10 μM) were added to the culture medium (100 μL per well) to treat cells for two additional days. Cell proliferation was quantified by using a Premix WST-1 Cell Proliferation Assay System (Takara, Japan). According to the manufacturer's protocol, cell proliferation was determined by measuring the optical density (OD) after the reaction for 3 hours by recording the absorbance at 450 nm using a plate reader (Multiskan Go, Thermo Fisher Scientific).

### FRET-Based 3CL^{pro} Enzyme Activity Assay

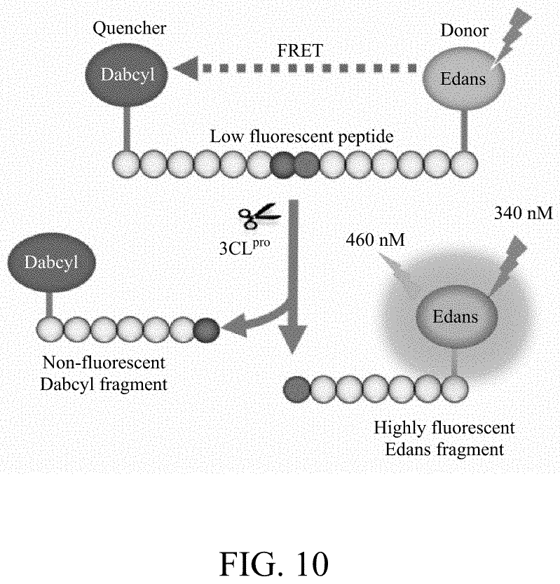

The FRET (fluorescence resonance energy transfer)-based assay was designed for 3CL^{pro} activity assay and explained in FIG. 10. The 3CL^{pro} enzyme assay was developed in a 96-well black microplate with a total volume of 50 μL. 30 μL of 0.5 ng/μL enzyme in a reaction buffer was added into each well, followed by the addition of 10 μL substrate solution to a final concentration of 40 μM. Enzymatic reaction gave fluorescence whose intensity was measured after incubation for 4 hours at 37° C. with slow shaking in a microtiter plate reading fluorimeter (BioTek Synergy HTX multi-mode reader, VT, US) with Ex=340 nm/Em=460 nm.

The following examples provide various non-limiting embodiments and properties of the present disclosure.

### Example 1: Virtual Screening Repurposed FDA-Approved Drugs as ATG4B-LC3B Recognition Interface Blockers

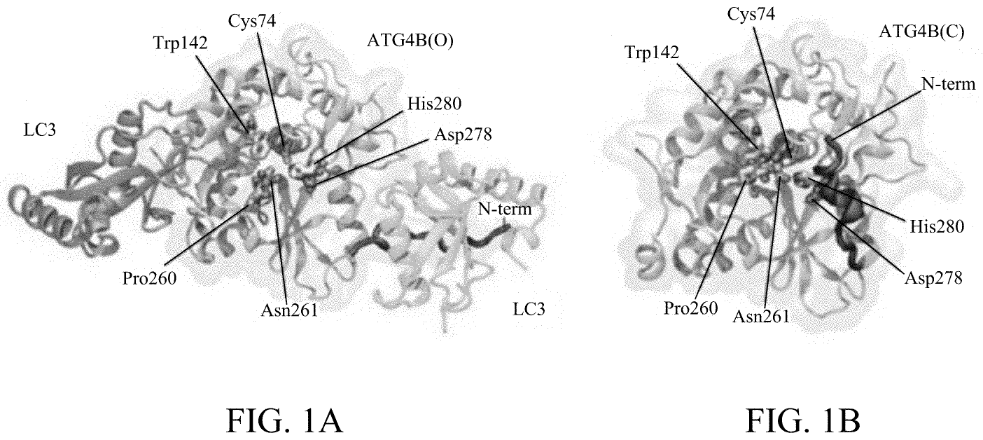

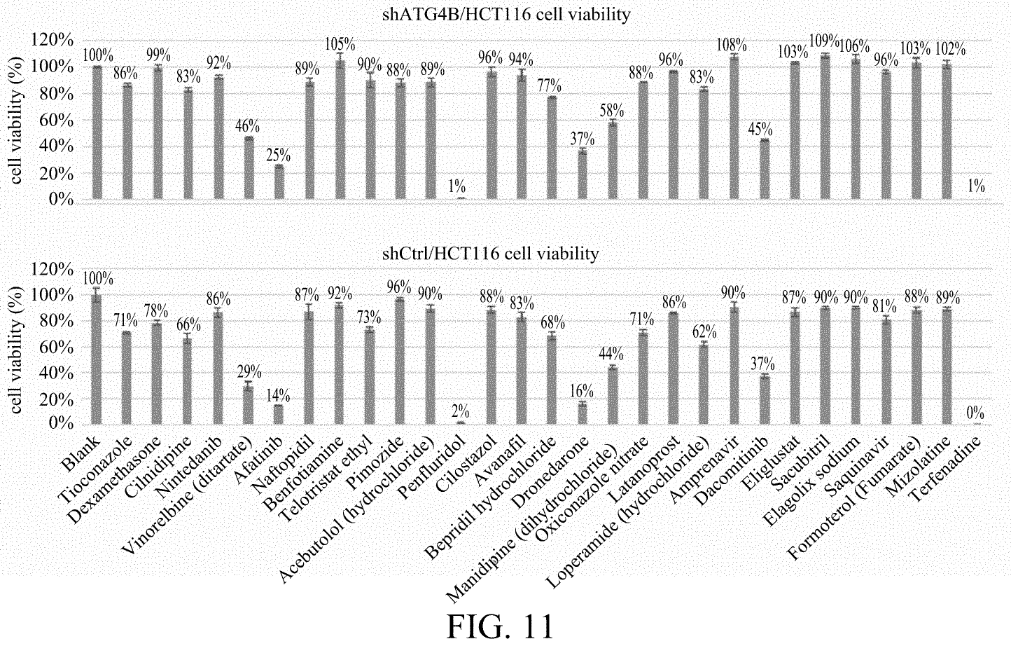

To identify potential drugs that could be repurposed as the ATG4B-LC3B recognition interface blockers, drugs were sought that had a strong association with the recognition interfaces of both ATG4B and LC3B. First, the residues that were in contact (heavy atoms within 4 Å) between ATG4B and LC3B in the crystallized ATG4B-LC3B complex structure (PDB code: 2Z0D) were defined as the recognition interfaces. The resulting interfaces were located at the residues surrounding the entry to the catalytic center (Cys74, Asp278, and His280) for ATG4B and the residues nearby the C-terminal tail for LC3B (FIG. 1A). Then, virtual screening was performed to dock 2016 FDA-approved drugs on both the interfaces of ATG4B and LC3B using AutoDock Vina. Vina output at most 20 docking poses for each drug dock to one protein, which produced 40307 poses on ATG4B surface and 40320 poses on LC3 surface. By estimating configurational entropy, drugs that cannot form large clusters were filtered out in the first stage. Out of 2016, 1183 and 928 drugs remained for ATG4B and LC3, respectively, of which only 667 drugs are in the intersection (FIG. 4A). Subsequently, top-ranked 100 drugs (Table 1) were selected based on their contacts with both proteins, and explicit-solvent MD simulations at body temperature and 1 bar pressure were performed for these drugs. Only 42 drugs remained bound on two proteins' interfaces after 10 ns simulations. The drugs were ranked by the sum of their binding free energy with each of the two proteins. The energetics and contact percentage after 10 ns for the top-ranked 30 drugs were listed (Table 2). One of the drugs, vinorelbine (Vb), ranked 19 by the sum of the binding free energies to ATG4B and LC3B calculated by MM/GBSA, originally as an anti-cancer drug for non-small cell lung cancer (NSCLC) by impairing the chromosomal segregation during mitosis (Gregory et al., 2000, British Journal of Cancer 82, 1907-1913), had ATG4B-dependent enhancement of the suppression on the colorectal cancer cell lines, HCT116 (FIG. 11), and showed 42% reduction of the ATG4B activity on LC3B catalysis in in vitro ATG4B cleavage assays (FIG. 4B). The virtual drug screening and ad hoc drug filtering and ranking methods of the present disclosure identified the anti-cancer drug, vinorelbine, as an ATG4B-LC3B recognition interface blocker, which could interfere with the catalysis of ATG4B on its substrate LC3B.

### Example 2: Vinorelbine Hindered the ATG4B-Mediated Catalysis of LC3B by Competing for the Enzyme-Substrate Binding Interface

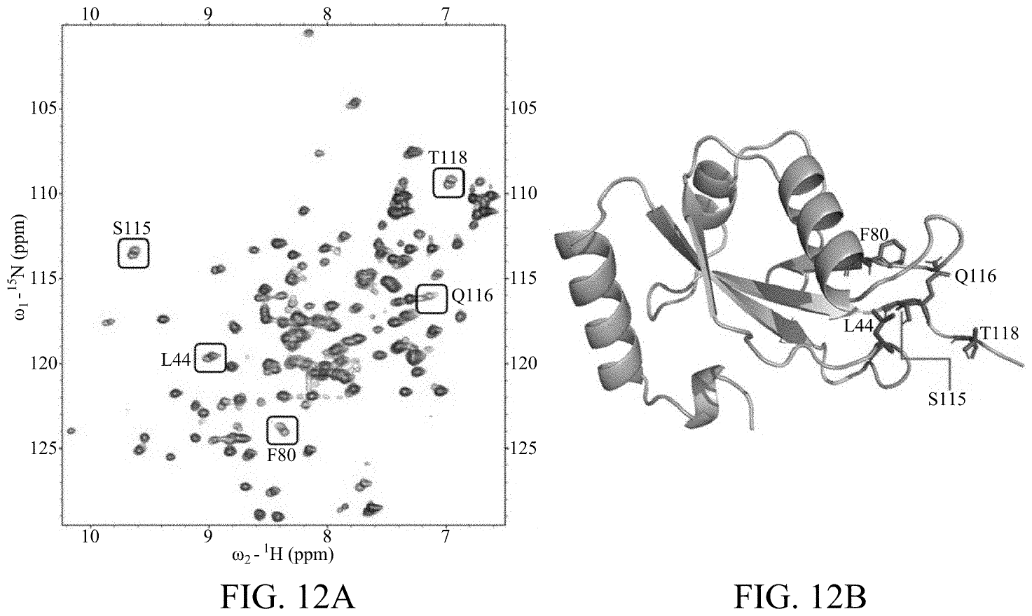

To confirm the molecular mechanism under the vinorelbine-mediated defect of ATG4B on LC3B catalysis, the binding site of vinorelbine on LC3B was examined by comparing the ¹H-¹⁵N HSQC spectra of LC3B with and without the titration of vinorelbine. When titrating the vinorelbine to LC3B in a 1:1 ratio, seven residues (Glu14, Leu44, Leu47, Phe80, Ser115, Gln116, and Thr118) were observed, showing large chemical shift perturbation (FIGS. 12A and 12B). These residues were overlapped with those involved in the binding interface of LC3B with ATG4B (FIGS. 2A and 2B). These results suggested that vinorelbine binds to the ATG4B-binding interface on LC3B as an interface blocker that can hinder the enzyme-substrate recognition and thus the catalysis of LC3B.

### Example 3: Vinorelbine Showed Autophagy-Dependent Toxicity to Cancer Cells Resulting in Reduced Autophagic Flux

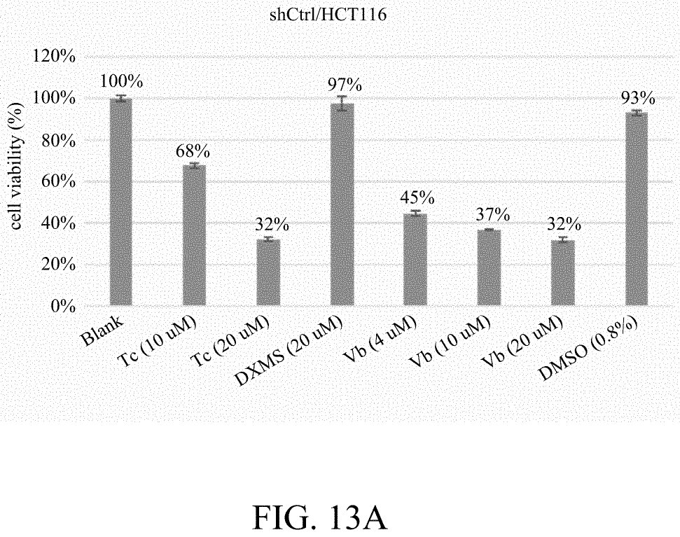

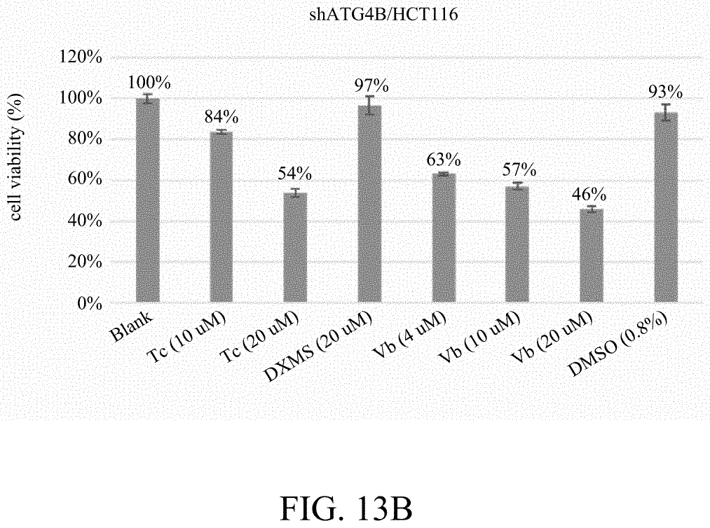

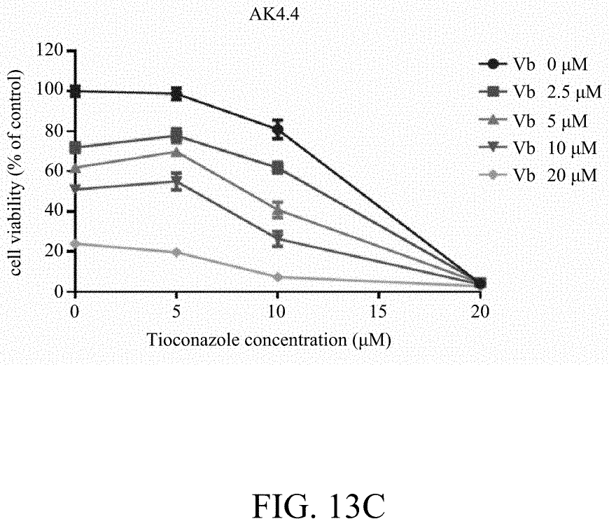

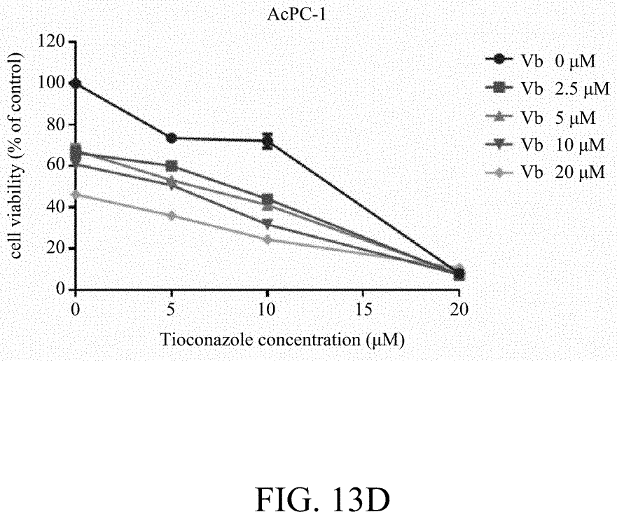

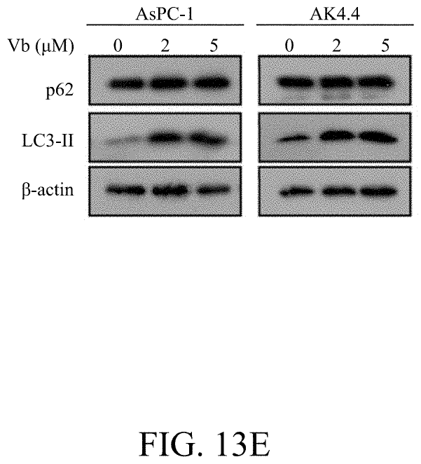

The cell toxicity of vinorelbine was further assayed by the cell viability assay on three cancer cell lines: human colon cancer HCT116, human pancreatic cancer AsPC-1, and mouse pancreatic cancer AK4.4. Vinorelbine showed effective toxicity to HCT116, with reduced 55%, 63%, and 68% of cell viability when treated in 4 μM, 10 μM, and 20 μM, respectively (FIG. 13A). Compared to that, vinorelbine showed 37%, 43%, and 54% of reduced cell viability in 4 μM, 10 μM, and 20 μM when treated in ATG4B knockdown (shATG4B) HCT116 (FIG. 13B). The decreased cell toxicity of vinorelbine in ATG4B knockdown HCT116 suggested the autophagy-dependent toxicity. For AK4.4 and AsPC-1, vinorelbine showed effective to moderate cell toxicity, with reduced 62%, 51%, 24% and 68%, 61%, 46% of cell viability when treated in 5 μM, 10 μM and 20 μM for AK4.4 and AsPC-1, respectively (FIGS. 13C and 13D). The abundance of LC3B-II in cells was quantified using western blotting. The treatment of vinorelbine resulted in the accumulation of LC3B-II, which suggested the attenuation of autophagy flux (FIG. 13E). Combining with the computation and NMR titration perturbation results, it suggested that vinorelbine can gain additional toxicity in autophagy-upregulated cancer cells by blocking the autophagy flux with accumulated LC3B-II due to the binding of vinorelbine as an interface blocker for the recognition and catalysis of ATG4B.

### Example 4: Co-Treatment of Tioconazole Additively and Synergistically Increased the Toxicity of Vinorelbine

Tioconazole, an FDA-approved drug originally for anti-fungi, has been found to inhibit the enzyme activity of ATG4B and to target the active site of ATG4B that blocks the entry of the substrate LC3B, which sensitizes the tumor to chemotherapy. It is interesting to see whether the combination of vinorelbine and tioconazole, with different underlying mechanisms for the impairment of ATG4B catalysis, can provide improved suppression of cancer cells. The cell viability assays were performed for AK4.4 and AsPC-1 treated by different combinations of the concentration of vinorelbine and tioconazole. To quantify the drug combination effect, the combination index (CI₅₀) was calculated as the sum of the factions of individual drugs' concentrations in combination to the IC₅₀ of the same drugs when 50% of the cell viability was reduced due to the drug combo (see Equation (1)). Smaller than or equal to unity refers to therapeutic synergy or additivity, respectively. For each tested concentration of vinorelbine, the increased concentration of tioconazole resulted in an enhanced reduction of the cell viability of AK4.4 (FIG. 13C). The CI₅₀ were 1.1 and 1.08 when the concentration of vinorelbine was controlled at 2.5 μM and 5 μM, respectively, which suggested that vinorelbine and tioconazole had an additive effect for the cell toxicity in a lower concentration of vinorelbine (Table 8). Specifically, Table 8 illustrates the combination index (CI) of the combined treatment of vinorelbine (Vb) and tioconazole (Tc) on mouse pancreatic cancer AK4.4. IC_{50,x}: inhibition concentration of drug x to cause 50% reduction of the cell viability. C_{x,50}: the drug concentration in combination for a 50% reduction of the cell viability.

TABLE 8
Combination index for co-treatment of Tc and Vb in AK4.4

| IC_{50, Vb} | IC_{50, Tc} | C_{Vb, 50} | C_{Tc, 50} | Combination index |
|---|---|---|---|---|
| (μM) | (μM) | (μM) | (μM) | (CI₅₀)¹ |
| 10.4 | 14.03 | 2.5 | 12.07 | 1.10 |
| | | 5 | 8.43 | 1.08 |
| | | 10 | 5.88 | 1.38 |

¹CI₅₀ is calculated based on Equation (1) in the Cell Viability section in MATERIALS AND METHODS

While not observing in AK4.4, a synergistic effect of vinorelbine and tioconazole was observed in AsPC-1, where combining 2.5 μM, 5 μM, and 10 μM of vinorelbine with the same corresponding concentration of tioconazole (2.5 μM, 5 μM, and 10 μM), resulting in 63%, 53%, and 32% cell viability that were lower than that of 68%, 61%, and 46% when 5 μM, 10 μM, and 20 μM vinorelbine alone was used, respectively (FIG. 13D). The synergistic effect was also reflected in the combination index, where the CI₅₀ were 0.75, 0.76 and 0.96 when vinorelbine was controlled at 2.5 μM, 5 μM and 10 μM, respectively (Table 9). These results suggested that the co-treatment of vinorelbine and tioconazole attenuated the autophagic flux and provided additive and minor synergistic effects on the toxicity toward cancer cell lines. Table 9 shows the combination index (CI) of the combined treatment of vinorelbine (Vb) and tioconazole (Tc) on human pancreatic cancer AsPC-1. The notations and calculations were the same as in Table 8.

TABLE 9
Combination index for co-treatment of Tc and Vb in AsPC-1

| IC_{50, Vb} | IC_{50, Tc} | C_{Vb, 50} | C_{Tc, 50} | Combination index |
|---|---|---|---|---|
| (μM) | (μM) | (μM) | (μM) | (CI₅₀) |
| 17.4 | 13.45 | 2.5 | 8.14 | 0.75 |
| | | 5 | 6.32 | 0.76 |
| | | 10 | 5.21 | 0.96 |

### Example 5: Treatment of Tioconazole Combined with Vinorelbine, TAT-N-Term-7, or TAT-N-Term-9 Enhanced the Suppression of Tumor Growth in the Mouse Xenograft Model

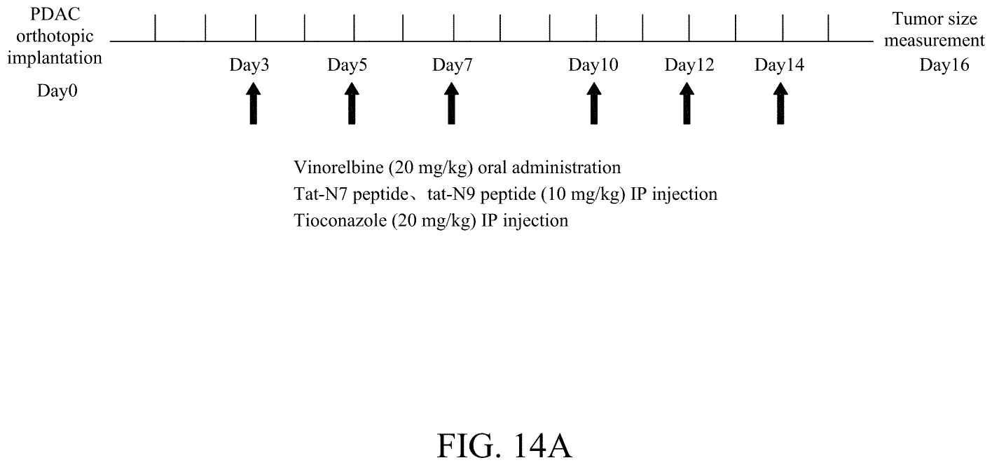

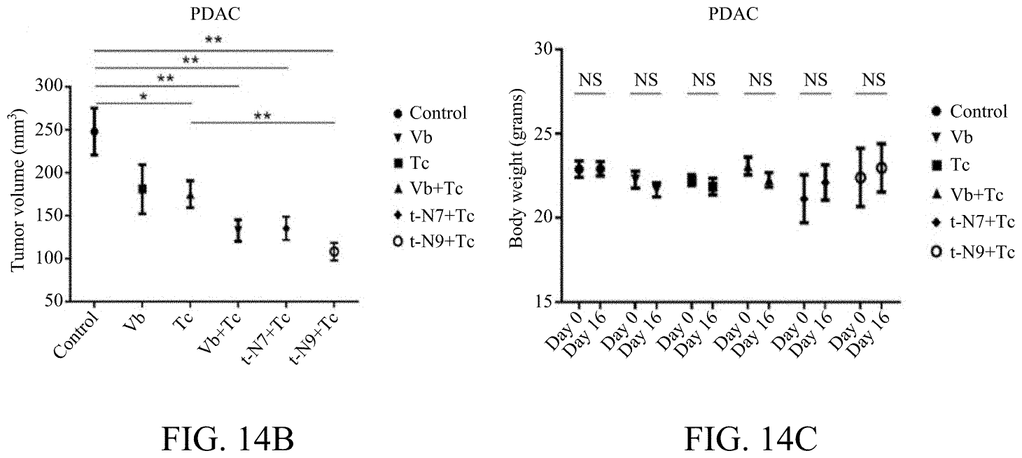

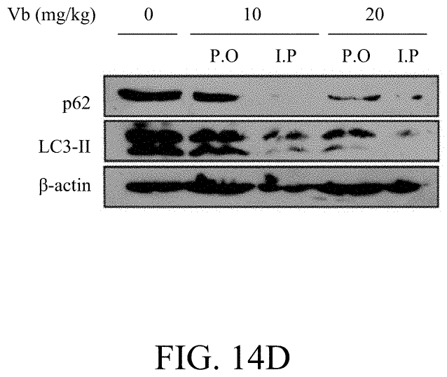

To assess the effect of the treatment of vinorelbine alone, tioconazole alone, or tioconazole combined with vinorelbine or with designed peptides for targeting LIR binding pocket on LC3B, i.e., TAT-N-term-7 (tat-N7, with an amino acid sequence of SEQ ID NO: 16 (YGRKKRRQRRR-GGS-YDTLGIF)) and TAT-N-term-9 (tat-N9, with an amino acid sequence of SEQ ID NO: 17 (YGRKKRRQRRR-GGS-YDTLRFAEF)), on tumors, the mouse tumor xenografts were established and subject to the treatment. Drugs of 5, 10 or 20 mg/kg were treated via pre-oral (P.O.) administration or intraperitoneal (I.P.) injection on days 3, 5, 7, 10, 12, and 14. In addition, 10 mg/kg tat-N7, 10 mg/kg tat-N9 or 20 mg/kg tioconazole were treated to the mouse via intraperitoneal (I.P.) injection. The mouse was sacrificed on day 16, and their body weights and tumor sizes were measured (FIG. 14A). Vinorelbine with 20 mg/kg effectively suppressed the tumor size to 70% of the size compared to the control group, which achieved a similar tumor suppression effect using the same amount of tioconazole. Also, it was found that the tumor size was significantly decreased more than 40% when treated with tioconazole combined with vinorelbine, TAT-N-term-7, or TAT-N-term-9, suggesting a synergistic effect of the combined treatment (FIG. 14B). The body weights of the mouse have no obvious difference in vinorelbine, tioconazole, and tioconazole combined with TAT-N-term-7 or with TAT-N-term-9 treatments, suggesting that all the treatments did not cause apparent side effects (FIG. 14C). The LC3B-II level in the tumor tissues was quantified by western blotting. However, the accumulation of LC3B-II due to the treatment of vinorelbine (FIG. 14D) was not observed. These results showed that vinorelbine, repurposed as an ATG4B-LC3B-II interface blocker, is a promising drug that can suppress tumor progression. Its effect can be further enhanced by co-treating with tioconazole, which leads to an additive and synergistic effect for the anti-ATG4B catalysis of LC3B-II and thus the growth of the tumor.

### Example 6: Treatment of Tioconazole Combined with TAT-N-Term-9 or TAT-N-Term-7 Showed Lower Toxicity as Compared with Tioconazole Alone

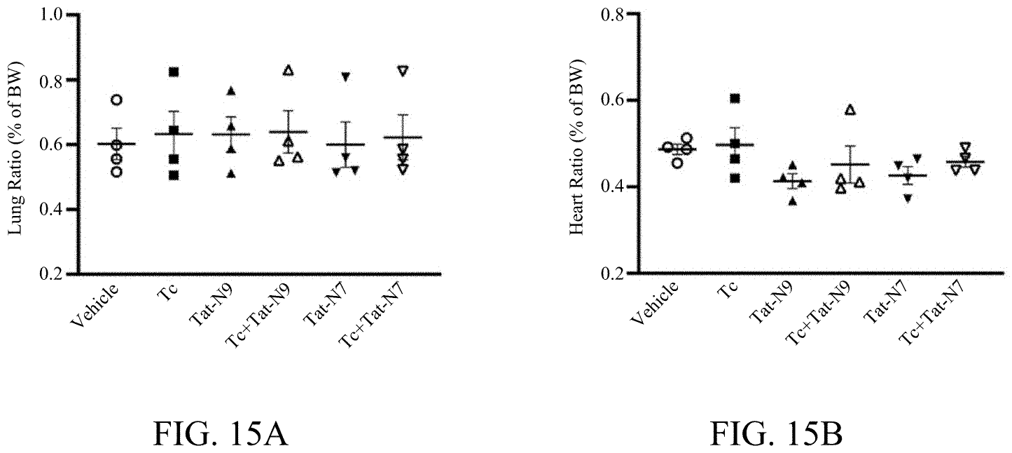

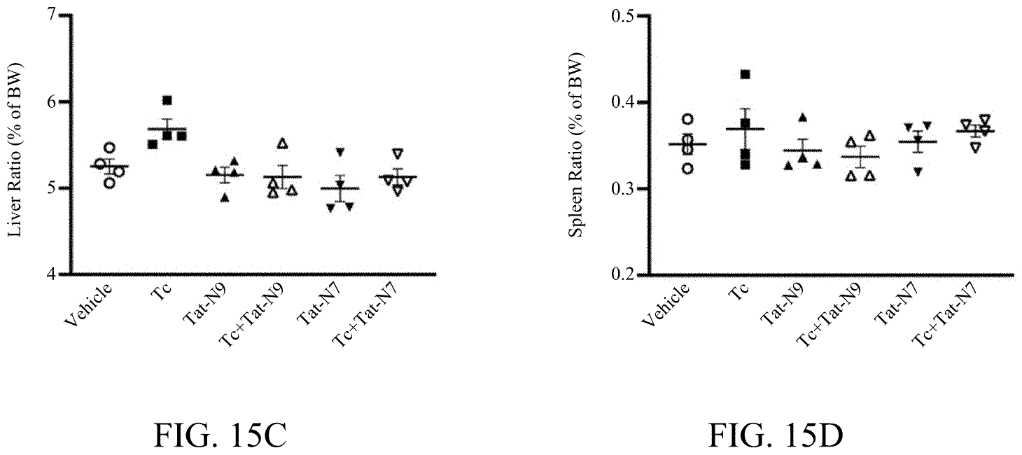

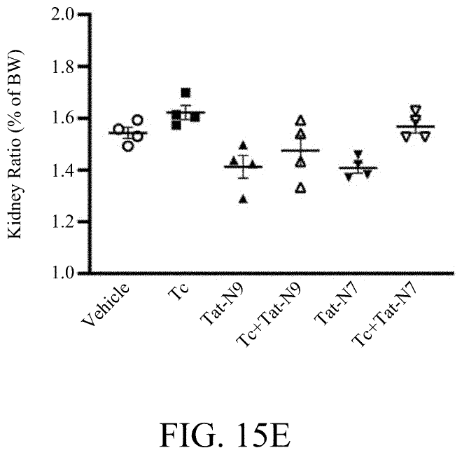

In order to access the possible toxicity of tioconazole (Tc), TAT-N-term-9 (Tat-N9), TAT-N-term-7 (Tat-N7), Tc combined with Tat-N9 (Tc+Tat-N9) or Tc combined with Tat-N7 (Tc+Tat-N7), these drugs are administered to healthy FVB/NJNarl mice via six intra-peritoneal (IP) injections throughout 16 days. Tc is a known active site inhibitor for ATG4B, and Tat-N9 and Tat-N7 are intermolecular allosteric drugs for ATG4B. It can be found that Tc caused an 8% increase (P-value =0.025) in the liver (from 5.25% to 5.69% of mouse body weight), while other treatment groups did not show the same effect (FIG. 15C). Biochemical data in the animal experimentation further revealed a 25% increase in triglyceride as compared to the control (from 159.1 to 198.2 mg/dL), which may imply the formation of fatty liver disease. Moreover, no swollen livers are found when adding allosteric peptide drug, Tat-N9 or Tat-N7, to Tc (FIG. 15C), and the combined treatment groups (Tc+Tat-N9 and Tc+Tat-N7) can reverse such an adverse effect of Tc evidenced by statistically equivalent sizes of organs between the control group and combined treatment groups (FIG. 15C). These data imply that the combinational use of the designed allosteric peptide drugs can be a safer option than a single treatment of active site drug, in addition to the demonstrated superior efficacy in combined treatment groups to the single use of active site drug Tc.

### Example 7: Repurposing FDA-Approved Drugs as the Modulators of LC3B Mediated Allosteric Regulation by Ensemble Docking

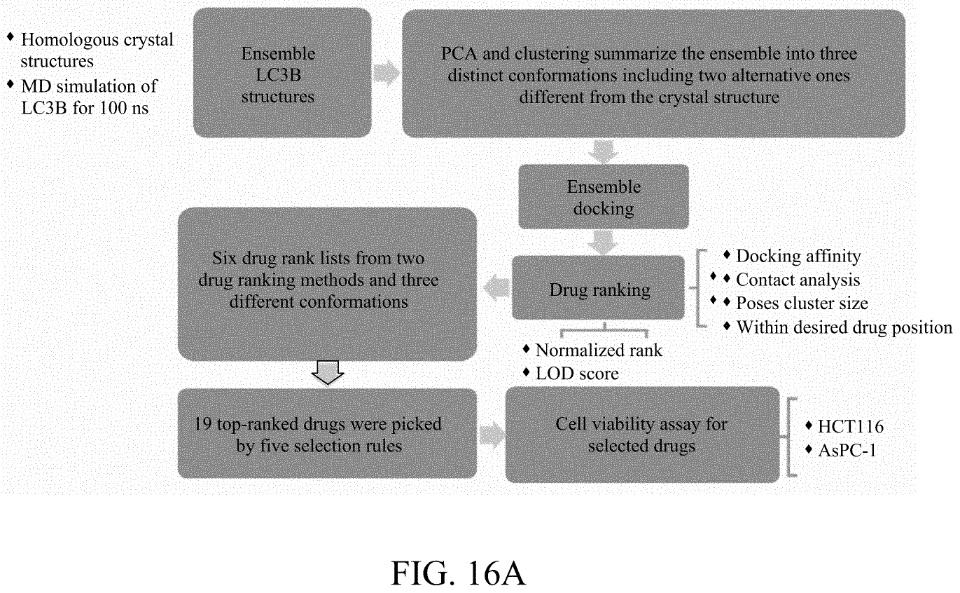

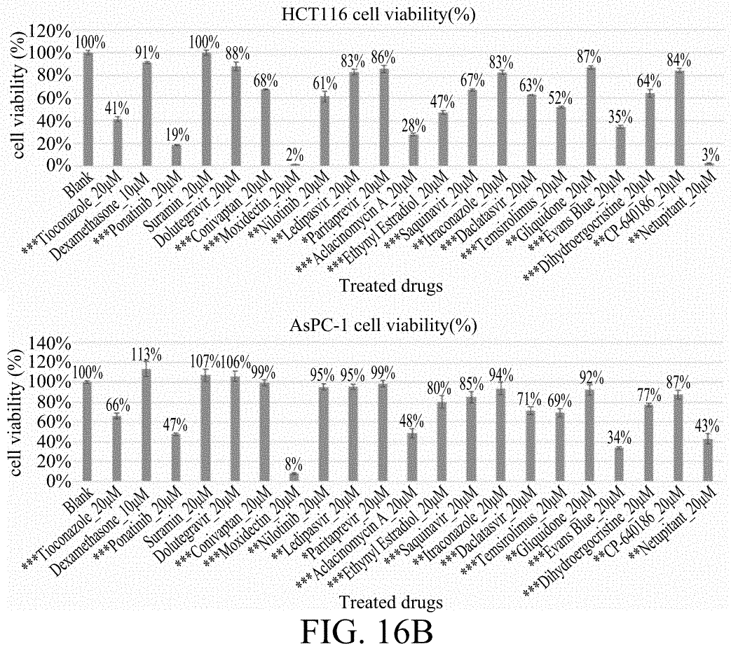

Besides as a substrate of ATG4B, the LC3B had a dual role as an intermolecular allosteric regulator to the activity of ATG4B. The allosteric regulation that could be interfered by the designed peptide inhibitors that competed the ATG4B N-terminal tail binding site on the LC3B required for the regulation. Accordingly, small molecular drugs that could reduce ATG4B activity through the same tricks were designed as the peptide inhibitors functioned. To find such drugs, ensemble docking strategy was applied to screen 2016 FDA-approved drugs. First, three representative alternative conformations of LC3B from crystalized structures of homology proteins were obtained and sampled for conformations in a 100 ns explicit solvent for MD simulation using the crystallized LC3B structure from PDB code, 2Z0DB, as the initial conformation. Principal component analysis and hierarchical clustering on these collected conformations resulted in three representative alternative conformations (FIG. 16A). Then, the 2016 drugs were docked on each of the three alternative conformations, and two different drug ranking methods, normalized ranking and logarithm of odds scoring (LOD scoring), were used to identify a promising drug considering the docking results from all the three alternative conformations. In the normalized rank method, drugs were ranked by the sum of the ranks transformed from the values of three different features, i.e., docking affinity (kcal/mol), number of contacts formed between docked pose and LC3B, and the extent of the pose located in the desired allosteric site. For the LOD scoring, the drugs were ranked based on the sum of the logarithm of odds, indicating the tendency that a sampled pose was resulted from a true binder or a decoy, derived from the three features, i.e., the distance to a specified target site on the allosteric binding site, the docking affinity, and the number of high-affinity poses in the pose cluster. A total of 19 highly ranked drugs (Table 10) were chosen based on the resulting six sets of drug ranking lists, for the two ranking methods and three alternative conformations, according to five selection criteria, and were tested for their suppression on the viability of cancer cell lines. Table 10 illustrates the selected top-ranked drugs, of which the inhibition of the cancer cells viability was tested on HCT116, AsPC-1, ATG4B-silencing strain of HCT116 (shATG4B/HCT116), and a corresponding control strain of HCT116 (shCtrl/HCT116).

TABLE 10

| | | | | HCT116 | AsPC-1 | shATG4B/ | shCtrl/ |
|---|---|---|---|---|---|---|---|
| | | | | Cell | Cell | HCT116 Cell | HCT116 Cell |
| | Conformation | Normal | LOD | Viability | Viability | Viability | Viability |
| Drug Name | Used | Rank | Rank | (%) | (%) | (%) | (%) |
| Ponatinib | X-ray | 1 | 500 | 18.5 ± 0.6 | 47.2 ± 1.3 | 31.0 ± 0.6 | 21.3 ± 0.6 |
| Suramin | X-ray | 25 | 1 | 100.1 ± 2.3 | 106.9 ± 6.5 | 98.3 ± 0.7 | 104.1 ± 4.4 |
| | Alternative | 13 | 11 | | | | |
| | conformation 1 | | | | | | |
| | Alternative | 6 | 29 | | | | |
| | conformation 2 | | | | | | |
| Dolutegravir | Alternative | 232 | 1 | 88.0 ± 4.0 | 105.8 ± 5.3 | 89.4 ± 2.5 | 85.5 ± 2.9 |
| | conformation 1 | | | | | | |
| Conivaptan | Alternative | 1 | 42 | 67.8 ± 0.5 | 99.3 ± 2.8 | 84.0 ± 2.0 | 69.5 ± 2.2 |
| | conformation 2 | | | | | | |
| Moxidectin | Alternative | 206 | 1 | 1.7 ± 0.1 | 7.8 ± 0.8 | 0.9 ± 0.1 | 1.0 ± 0.1 |
| | conformation 2 | | | | | | |
| Nilotinib | X-ray | 2 | 501 | 61.4 ± 4.4 | 95.2 ± 3.4 | 68.2 ± 1.1 | 59.6 ± 1.7 |
| | Alternative | 15 | 13 | | | | |
| | conformation 1 | | | | | | |
| | Alternative | 8 | 64 | | | | |
| | conformation 2 | | | | | | |
| Ledipasvir | X-ray | 10 | 2 | 82.9 ± 2.7 | 95.2 ± 2.9 | 90.2 ± 1.8 | 72.5 ± 0.8 |
| | Alternative | 248 | 14 | | | | |
| | conformation 1 | | | | | | |
| | Alternative | 15 | 50 | | | | |
| | conformation 2 | | | | | | |
| Paritaprevir | Alternative | 2 | 18 | 85.8 ± 3.3 | 98.7 ± 3.0 | 88.0 ± 1.4 | 75.4 ± 1.3 |
| | conformation 1 | | | | | | |
| Aclacinomycin A | X-ray | 21 | 8 | 27.9 ± 1.3 | 48.3 ± 4.6 | 38.4 ± 1.3 | 27.7 ± 1.2 |
| | Alternative | 89 | 2 | | | | |
| | conformation 1 | | | | | | |
| Ethynyl Estradiol | Alternative | 2 | 276 | 47.4 ± 1.8 | 80.1 ± 6.4 | 69.5 ± 3.3 | 48.7 ± 1.4 |
| | conformation 2 | | | | | | |
| Saquinavir | X-ray | 3 | 43 | 67.0 ± 1.2 | 84.6 ± 5.5 | 65.4 ± 1.2 | 63.5 ± 0.8 |
| Itraconazole | Alternative | 3 | 7 | 82.6 ± 2.1 | 93.6 ± 6.3 | 91.3 ± 2.3 | 84.1 ± 1.9 |
| | conformation 1 | | | | | | |
| Daclatasvir | Alternative | 47 | 3 | 62.7 ± 0.3 | 71.1 ± 4.2 | 69.4 ± 3.7 | 58.8 ± 2.6 |
| | conformation 1 | | | | | | |
| Temsirolimus | Alternative | 167 | 3 | 52.1 ± 0.9 | 69.0 ± 4.2 | 60.5 ± 1.9 | 49.5 ± 3.5 |
| | conformation 2 | | | | | | |
| Gliquidone | X-ray | 5 | 4 | 87.0 ± 1.6 | 92.4 ± 5.3 | 89.6 ± 0.6 | 88.4 ± 7.3 |
| Evans Blue | X-ray | 9 | 7 | 34.8 ± 1.3 | 33.6 ± 1.2 | 34.5 ± 1.7 | 34.9 ± 0.3 |
| | Alternative | 161 | 9 | | | | |
| | conformation 1 | | | | | | |
| | Alternative | 56 | 5 | | | | |
| | conformation 2 | | | | | | |
| Dihydroergocristine | X-ray | 17 | 3 | 64.0 ± 3.7 | 77.1 ± 1.6 | — | — |
| CP-640186 | X-ray | 19 | 499 | 84.3 ± 2.0 | 87.4 ± 4.7 | — | — |
| | Alternative | 22 | 12 | | | | |
| | conformation 1 | | | | | | |
| Netupitant | Alternative | 20 | 32 | 2.8 ± 0.4 | 42.9 ± 5.1 | — | — |
| | conformation 2 | | | | | | |

The suppression of drugs for the cancer cell viability was tested on the colorectal cancer, HCT116, and pancreatic cancer, AsPC-1, cell lines. For HCT116, the remaining cell viability upon treatment of 20 μM ponatinib (19%), moxidectin (2%), aclacinomycin A (28%), ethynyl estradiol (47%), Evans blue (35%), and netupitant (3%) showed more than 50% inhibition of the cell viability (FIG. 16B). Among them, moxidectin, aclacinomycin A, ethynyl estradiol, and netupitant were identified from the drug screening by docking using the sampled alternative conformations of LC3B.

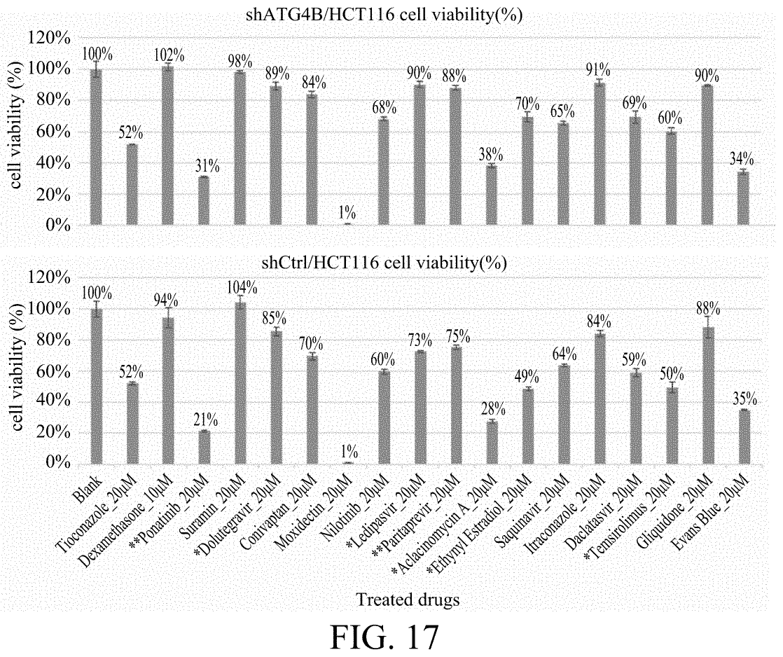

To see whether the observed inhibition of cell viability of the drugs for HCT116 was resulted from the perturbation of ATG4B-LC3B regulatory mechanism and thus the autophagy pathway, an ATG4B silencing strain (shATG4B/HCT116, S strain) and a control strain (shCtrl/HCT116, C strain) were cultured for the cell viability assays of picked drugs. From the resulted remaining cell viability, it showed that 20 μM of ponatinib (S strain: 31%, C strain: 21%), moxidectin (S strain: 1%, C strain: 1%), aclacinomycin A (S strain: 38%, C strain: 28%), ethynyl estradiol (S strain: 70%, C strain: 49%), temsirolimus (S strain: 60%, C strain: 50%), and Evans blue (S strain: 34%, C strain: 35%) could effectively inhibit the cell viability (FIG. 17). Temsirolimus, similar as moxidectin, aclacinomycin A and ethynyl estradiol, was identified using the sampled alternative conformation of LC3B in the docking simulation of drug screening. Among the tested drugs, ponatinib, dolutegravir, ledipasvir, paritaprevir, aclacinomycin A, ethynyl estradiol, and temsirolimus showed a statistically significant (p-value <0.05) reduction of the inhibition effects in the ATG4B silencing strain (S strain) as compared to the control strain (C strain), suggesting that the inhibition effects were partly come from the perturbation of the ATG4B-LC3B regulatory system (FIG. 17). When treating similar concentration of drugs (20 μM) on AsPC-1 pancreatic cell lines, the remaining cell viability of ponatinib, moxidectin, aclacinomycin A, ethynyl estradiol, daclatasvir, temsirolimus, Evans blue, netupitant, and dihydroergocristine showed statistically significant (p-value <0.05) inhibition of cell viability, where ponatinib (47%), moxidectin (8%), aclacinomycin A (48%), Evans blue (34%), and netupitant (43%) showed less than 50% of the remaining cell viability (FIG. 16B). These results showed that several FDA-approved drugs identified by the virtual drug screening from conformational ensemble can effectively inhibit the viability of both the cancer cell lines of HCT116 and AsPC-1 and could be promising anti-cancer drugs functioned as the allosteric drugs of ATG4B.

### Example 8: Ponatinib and Moxidectin can be Inter-Molecular Allosteric Inhibitors of ATG4B which Additively Enhance the Treatment Effect of Tioconazole or Vinorelbine

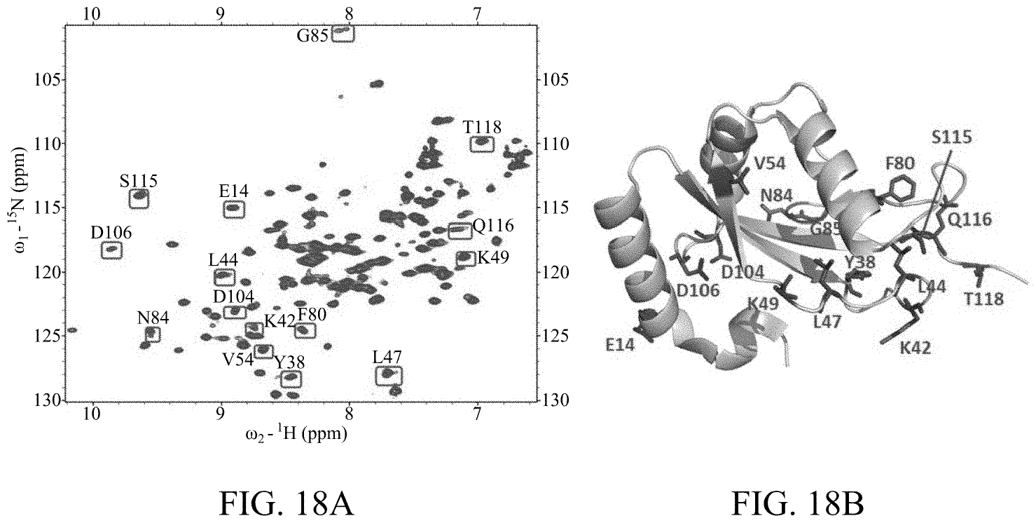

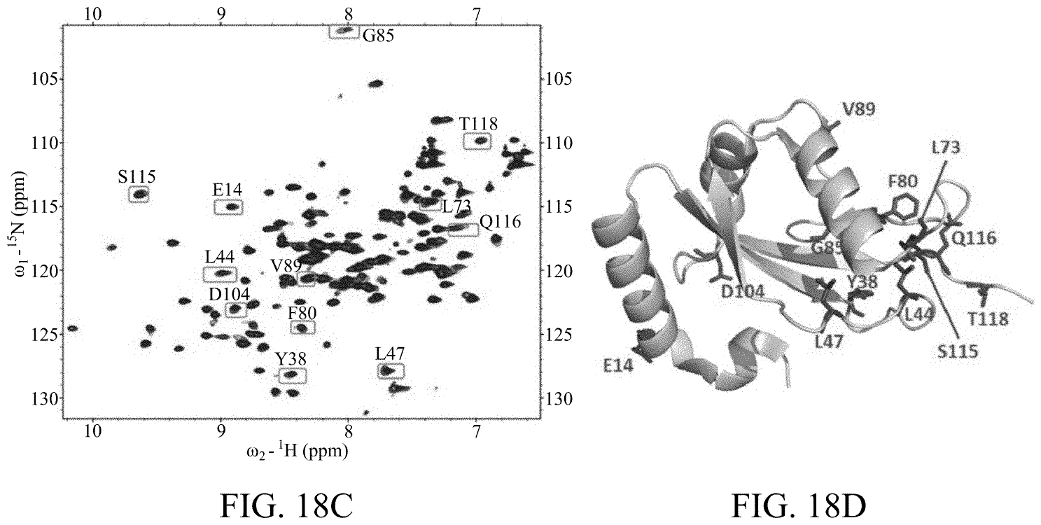

Among the above-identified drugs, ponatinib and moxidectin were chosen for further assays due to their consistent inhibition on the viability of cancer cells. The binding site of ponatinib or moxidectin on LC3B was examined by comparing the ¹H-¹⁵N HSQC spectra of LC3B with and without the titration of ponatinib or moxidectin, and the results were shown in FIGS. 18A to 18D.

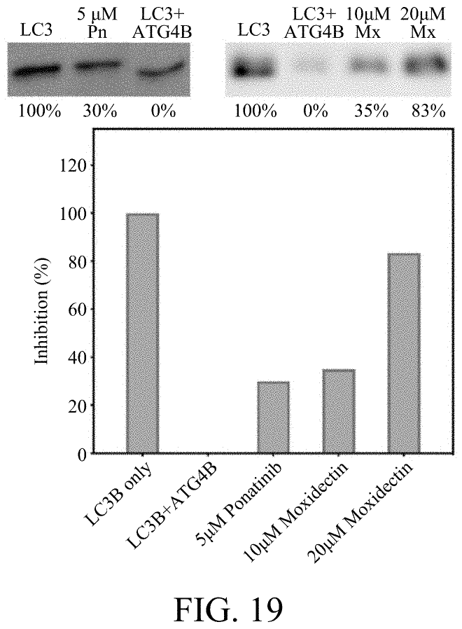

Further, to confirm the molecular origin of their cell toxicity, the in vitro cleavage assays were performed. As expected, both ponatinib (Pn) and moxidectin (Mx) showed moderate inhibition of 30% and 38% on ATG4B's catalytic activity at 5 μM and 10 μM, respectively (FIG. 19), which could result from the modulation of the allosteric regulation by competing the ATG4B N-terminal tail binding site on LC3B.

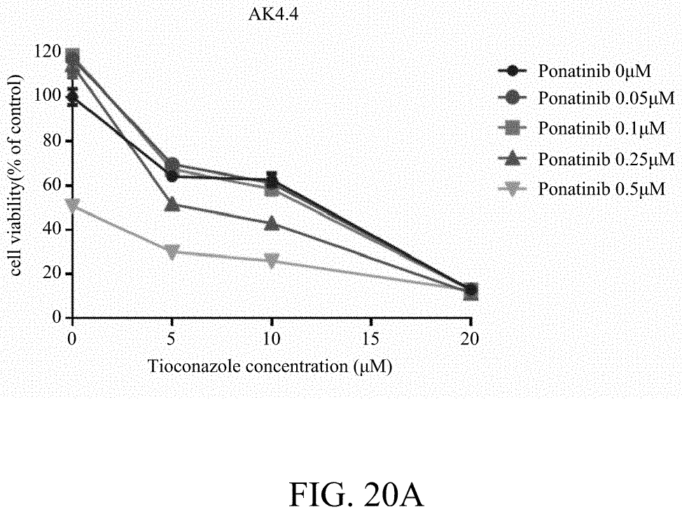

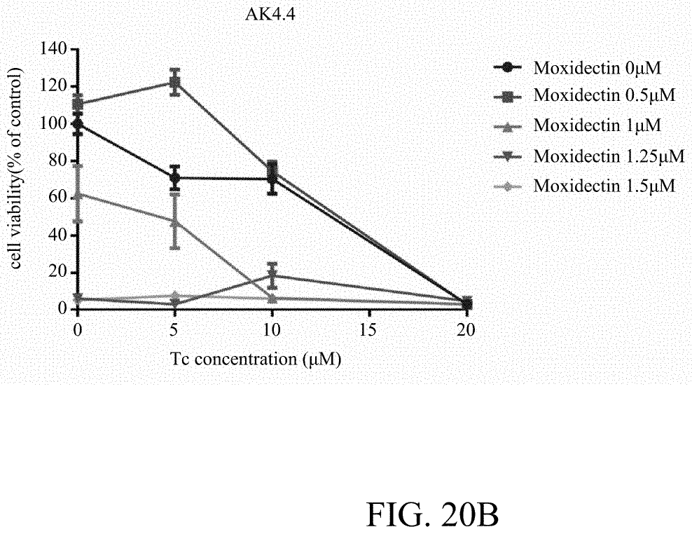

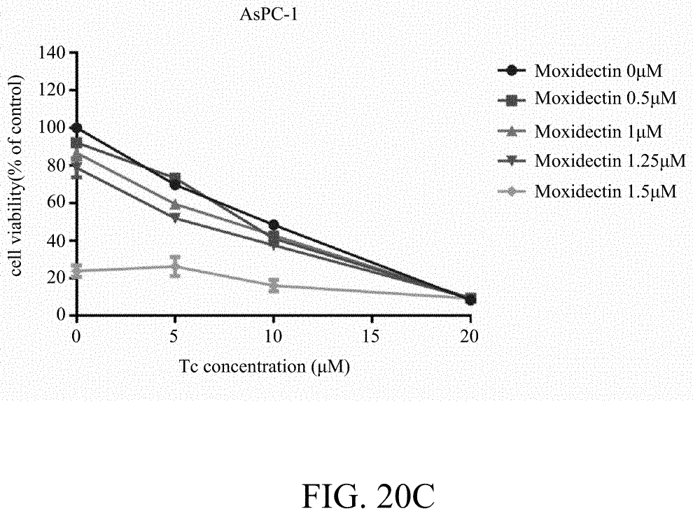

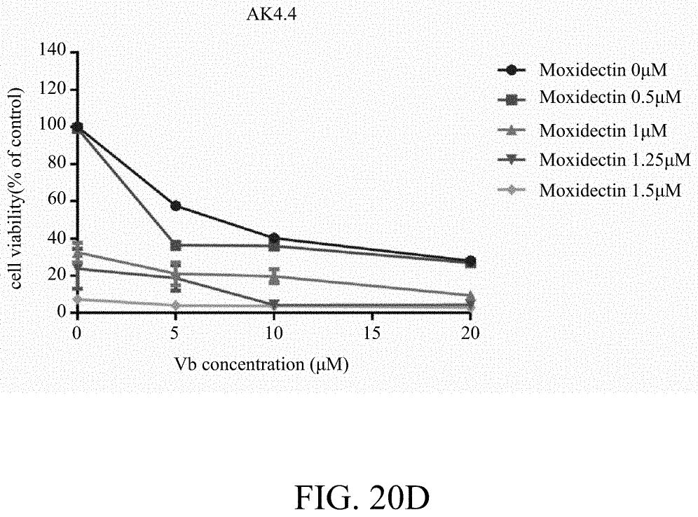

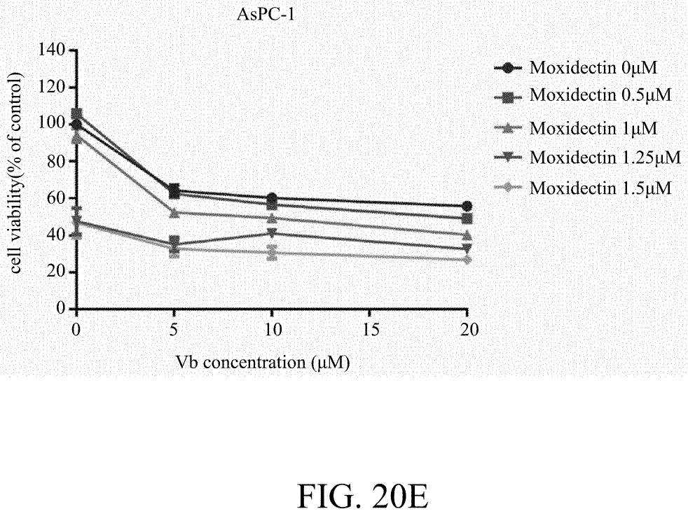

Furthermore, the allosteric inhibition of ponatinib or moxidectin can be additively combined with the known orthosteric inhibitor, tioconazole, to suppress the viability of cancer cells, AK4.4 (FIGS. 20A and 20B), with the CIs for different concentrations of drug combination nearly equal to unity (Table 11 and Table 12). Table 11 shows the combination index (CI) of the combined treatment of the allosteric inhibitor, ponatinib (Pn), and tioconazole (Tc) on mouse pancreatic cancer AK4.4, and Table 12 shows the combination index (CI) of the combined treatment of the allosteric inhibitor, moxidectin (Mx), and tioconazole (Tc) on mouse pancreatic cancer AK4.4. The notations and calculations were the same as those in Table 8. It was found that ponatinib and moxidectin can be repurposed as the inter-molecular allosteric inhibitors of ATG4B, which can be combined with ATG4B's orthosteric drug to give an additive effect on AK4.4. The similar synergistic effect of moxidectin (Mx) combined with tioconazole (Tc) can be found on human pancreatic cancer AsPC-1. It can be found that when combined with tioconazole (0.1 μM or 0.5 μM), the treatment of moxidectin can further suppress the growth of AsPC-1 (FIG. 20C and Table 13).

TABLE 11
Combination index for co-treatment of
ponatinib and tioconazole in AK4.4

| IC_{50, Pn} | IC_{50, Tc} | C_{Pn, 50} | C_{Tc, 50} | Combination Index |
|---|---|---|---|---|
| (μM) | (μM) | (μM) | (μM) | (CI₅₀) |
| 0.51 | 12.54 | 0.05 | 12.31 | 1.08 |
| | | 0.1 | 11.85 | 1.14 |
| | | 0.25 | 5.86 | 0.96 |
| | | 0.5 | 0.17 | 0.99 |

TABLE 12
Combination index for co-treatment of
moxidectin and tioconaozle in AK4.4

| IC_{50, Mx} | IC_{50, Tc} | C_{Mx, 50} | C_{Tc, 50} | Combination Index |
|---|---|---|---|---|
| (μM) | (μM) | (μM) | (μM) | (CI₅₀) |
| 1.07 | 13.03 | 0.50 | 13.42 | 1.50 |
| | | 1.00 | 4.21 | 1.26 |

TABLE 13
Combination index for co-treatment of
moxidectin and tioconaozle in AsPC-1

| IC_{50, Mx} | IC_{50, Tc} | C_{Mx, 50} | C_{Tc, 50} | Combination Index |
|---|---|---|---|---|
| (μM) | (μM) | (μM) | (μM) | (CI₅₀) |
| 1.38 | 9.61 | 0.50 | 8.61 | 1.26 |
| | | 1.00 | 7.83 | 1.54 |
| | | 1.25 | 5.65 | 1.49 |

In addition, the allosteric inhibition of moxidectin (Mx) can be additively combined with the interface blocker, vinorelbine (Vb), to suppress the viability of cancer cells, AK4.4 and AsPC-1 (FIGS. 20D and 20E), with the CIs for different concentrations of drug combination shown in Table 14 and Table 15 below.

TABLE 14
Combination index for co-treatment of
moxidectin and vinorelbine in AK4.4

| IC_{50, Mx} | IC_{50, Vb} | C_{Mx, 50} | C_{Vb, 50} | Combination Index |
|---|---|---|---|---|
| (μM) | (μM) | (μM) | (μM) | (CI₅₀) |
| 0.87 | 7.19 | 0.50 | 3.91 | 1.12 |

TABLE 15
Combination index for co-treatment of
moxidectin and vinorelbine in AsPC-1

| IC_{50, Mx} | IC_{50, Vb} | C_{Mx, 50} | C_{Vb, 50} | Combination Index |
|---|---|---|---|---|
| (μM) | (μM) | (μM) | (μM) | (CI₅₀) |
| 1.24 | 33.10 | 0.50 | 18.94 | 0.98 |
| | | 1.00 | 9.05 | 1.08 |

### Example 9: In Silico Designed Peptides Mimicking ATG4B N-Terminus for LC3B Binding Showed Moderate Inhibition of ATG4B Enzyme Activity

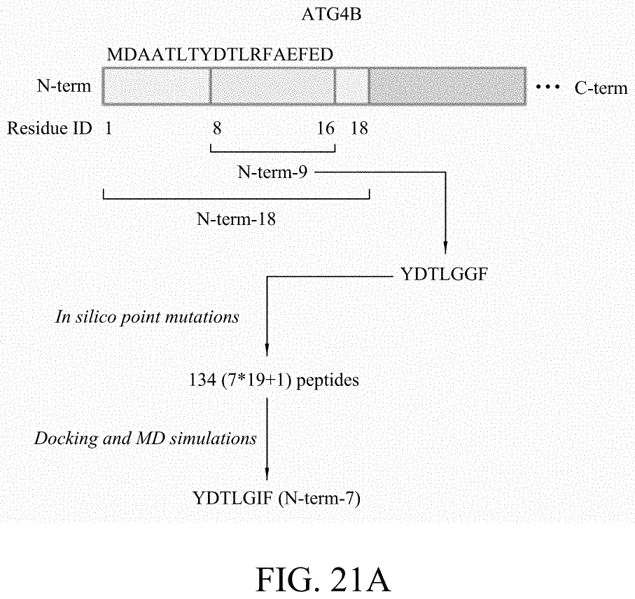

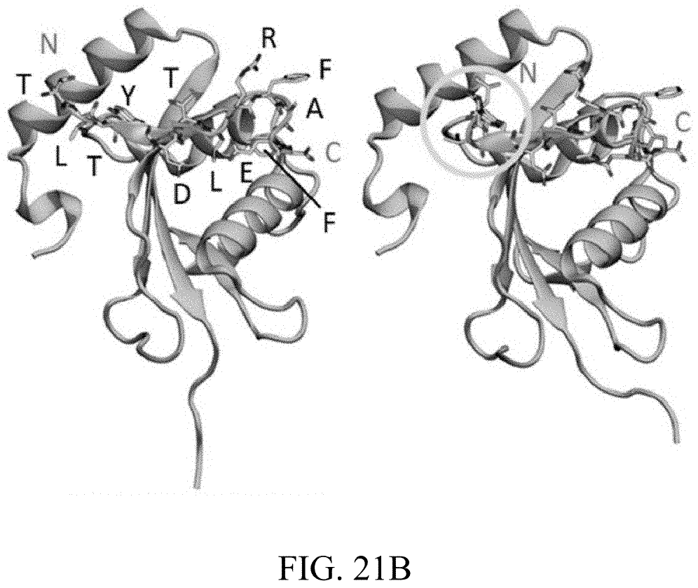

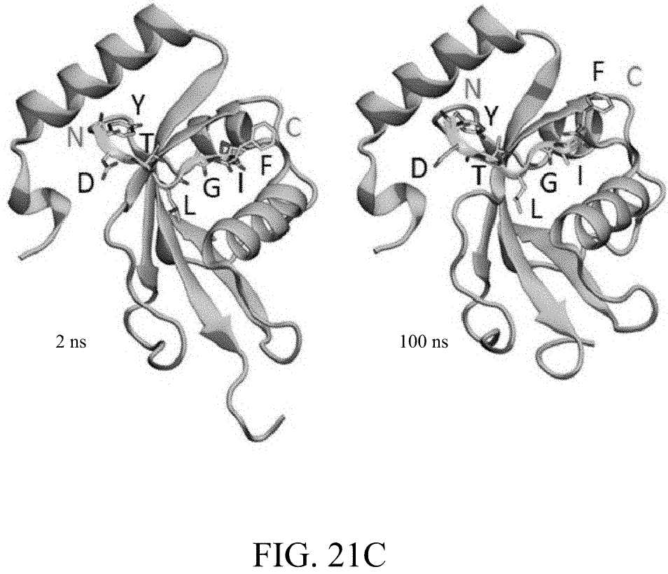

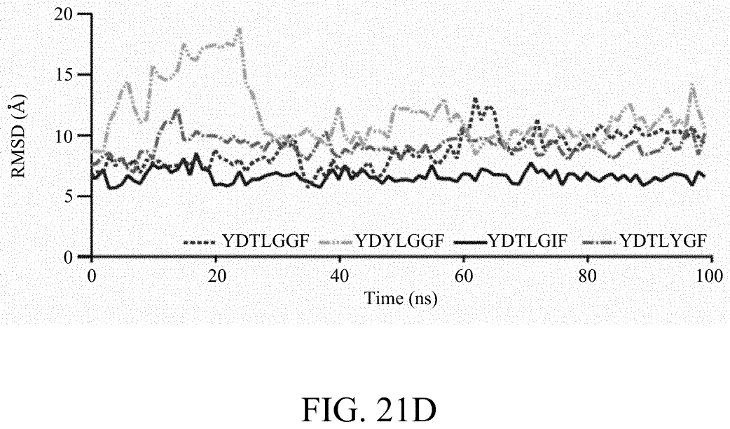

The existence of the LC3B-induced intermolecular allosteric regulation of ATG4B can be evaluated by designing peptides that compete with ATG4B's N-terminus for the binding of LC3B. Any decrease of the LC3B-enhanced ATG4B enzyme activity in the presence of these peptides can be interpreted as the existence of positive cooperativity of LC3B-mediated allosteric regulation. Three synthesized peptides were tested. The first was taken from the first 18 residues of ATG4B N-terminus (N-term-18, SEQ ID NO: 19 (MDAATLTYDTLRFAEFED), or N18), which has been shown to interact with LC3B and caused the chemical shift perturbation on the ATG4B N-terminus binding site (also termed LC3-interacting region (LIR) binding site) in previous NMR titration experiments (FIG. 21A and FIG. 21). The second peptide, as part of N-term-18, was determined by both crystal structure and MD simulation results. In the crystal structure of open-form ATG4B (PDB code: 2Z0D), the interaction between ATG4B N-terminal tail and the LIR binding pocket on LC3B can be found in an adjacent asymmetric unit (FIG. 21A). Within the ATG4B N-terminal tail, i.e., N-term-18, the first 4 residues, MDAA, could not be solved by x-ray, and the last two residues, ED, did not show any contact with N-LC3 in the crystal structure. The truncated N-term-18 with the removal of MDAA and ED in the N- and C-terminus, respectively, was termed as N-term-12, SEQ ID NO: 18 (TLTYDTLRFAEF). In a 30 ns MD simulation for the ATG4B and N-term-12 complex, the first three residues “TLT” were found to have enhanced fluctuations while the “L” largely lose contact with LC3B (FIG. 21B and Table 5). Therefore, the remaining part for binding as the second peptide (N-term-9, SEQ ID NO: 20 (YDTLRFAEF), or N9) (FIG. 21A) that actually contains the LIR (i.e., YDTL).

To further design a peptide with a higher binding affinity to compete with ATG4B N-terminal tail as the third peptide, the four residues (RFAE) in the middle of the N-term-9 peptide (SEQ ID NO: 20 (YDTLRFAEF)) that were found to be more flexible (root-mean-square fluctuation (RMSF)>1 Å) than other N9 residues (Table 5) during the simulation were removed. The last residue (Phe 16) was retained for its increased contact with LC3B over the 30 ns simulation (Table 5). To re-join the topologically disconnected “YDTL” and “F,” a short linker of glycine (G) repeats was inserted to replace the 4 (RFAE) residues in silico. The length of the linker should cover at least the linear distance between Leul 1 and Phe16 that is around 8 Å.

To determine a suitable length of the linker, the number of native contacts was defined as those heavy atoms of LC3B within 4 Å from the fourth to seventh (YDTL) and last (F) residues of N-term-12 (YDTL) and eleventh (F) residue of N-term-12 in the crystal structure. The residues, Asp19, Ile23, Lys30, Lys49, Lys51, Phe52, Leu53, Va154, Pro55, Va158, Glu62, Leu63, Ile66, Arg70, and Phe108, were found to contact “YDTL” and “F” in the N-term-12, among which the residues, Lys51, Phe52, Leu53, Va154, and Va158, were found perturbed in LC3B's ¹H-¹⁵N HSQC spectra in the presence of the synthesized N-term-18 with Δp.p.m. >0.2. Then, the retaining native contact ratio for the “YDTL” and “F” was calculated in each of these docked peptides, i.e., SEQ ID NO: 21 (YDTLGF), SEQ ID NO: 14 (YDTLGGF) and SEQ ID NO: 22 (YDTLGGGF), and it was found that the peptide with a GG linker gave a docking pose that recovered the native contact, with a ratio of 0.84 (Table 6). Also, the Cα distance between Leu11 and Phe16 was 8.14 Å, longer than the double of the average Cα distance (3.8 Å), which also suggested the introduction of a GG linker that allowed extending to at least triple of the average Cα distance.

To further optimize the peptide with a double G linker (SEQ ID NO: 14 (YDTLGGF)), point mutation on each residue was performed to the other 19 residues, which resulted in totally 134 (=7×19+1) peptides, including SEQ ID NO: 14 (YDTLGGF). The generated peptides were then subject to docking screening by AutoDock Vina. All the resulting poses were first compared to N-term-18 in the x-ray structure by calculating the Cα atom RMSD of YDTL and F residues in the observed N-term-12 and those in the docking poses of the aforementioned three peptides. The docking poses that were similar enough to the N-terminal tail (RMSD <10 Å) were retained and ranked based on AutoDock Vina-predicted binding affinities (Table 7). The top-ranked three peptides (SEQ ID NO: 1 (YDYLGGF), SEQ ID NO: 2 (YDTLGIF), SEQ ID NO: 3 (YDTLYGF), Table 7, upper, indicated by asterisk) and the most top-ranked pose of SEQ ID NO: 14 (YDTLGGF) (Table 7, bottom, indicated by asterisk), as a control, were selected for further 100-ns simulations to assess their binding stabilities. The simulation results suggested that SEQ ID NO: 2 (YDTLGIF) was a promising candidate to form stable interactions with LC3B as the binding of this peptide that was very stable through the 100 ns simulation with the docking pose of the last snapshot remained similar from 2 ns to the end of the simulation (FIGS. 21C and 21D), and the docking poses in the trajectory resembled to the N-terminal tail, based on the RMSD values calculated as described above, when compared to the other three (FIG. 21D). For SEQ ID NO: 1 (YDYLGGF), the N-terminus totally left the binding pocket after 100 ns simulation (FIG. 21D). For SEQ ID NO: 3 (YDTLYGF), the peptide was unstable through the simulation and slightly left the binding pocket at the end of the simulation (FIG. 21D). For the template SEQ ID NO: 14 (YDTLGGF), it was not as stable as SEQ ID NO: 2 (YDTLGIF) in the 100 ns simulation (FIG. 21D). SEQ ID NO: 2 (YDTLGIF), coined as N-term-7 thereafter, was taken as the third peptide that can potentially compete with N-terminus of ATG4B for LC3B binding.

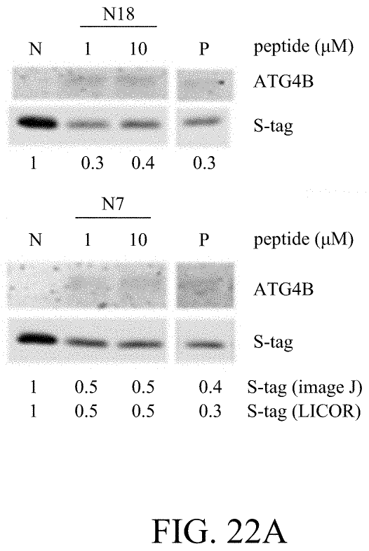

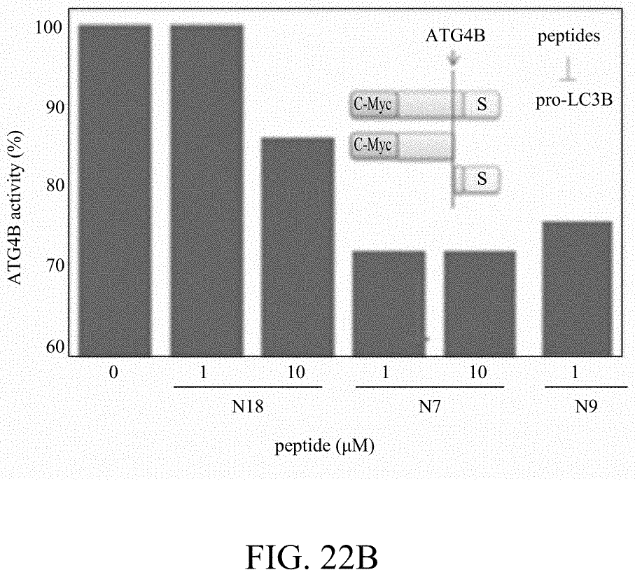

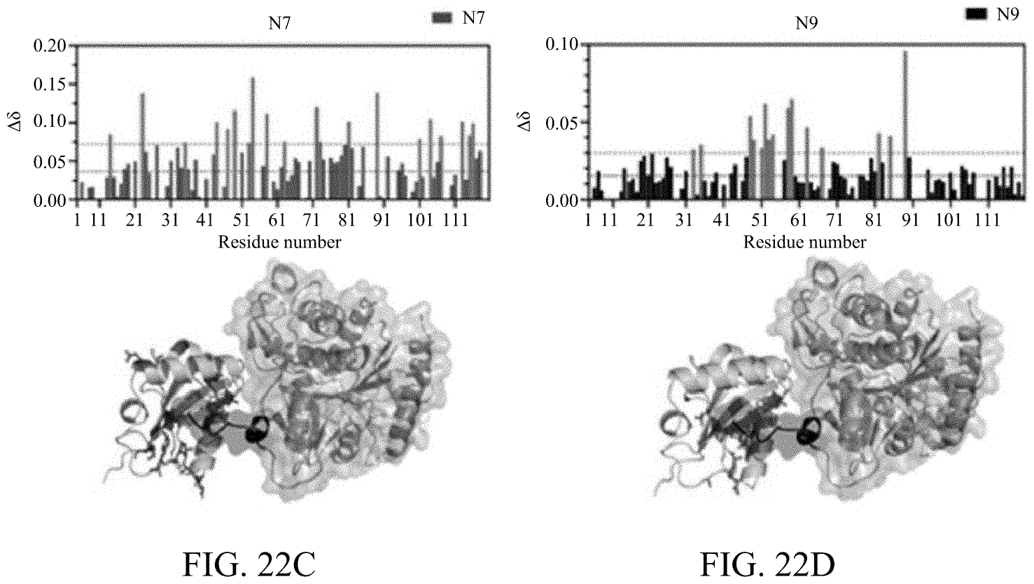

The inhibitory effect of the three designed peptides on ATG4B activity was tested using ATG4B cleavage assays and ATG4B activity reporter assays. In the ATG4B cleavage assays, a peptide that can inhibit ATG4B activity will result in darker bands of the full-length fusion protein composed of C-Myc, pro-LC3B, and S-tag, which can be quantified by immunoblotting for S-tag and C-Myc. The full-length LC3B fusion protein was used as the negative control and as the positive control when also incubated with ATG4B (FIG. 22A). The ATG4B activity reporter assays were performed as described in the previous study (Shu et al., 2010, Autophagy 6, 936-947). A peptide that can inhibit ATG4B activity will weaken the fluorescence signal produced by the cleaved PLA2. The results showed that both N-term-18 assayed by ATG4B cleavage assays and N-term-9 assayed by ATG4B activity reporter assays had moderate inhibition to ATG4B activity when compared to the controls (about 15% for N-term-18 and 25% for N-term-9 at 10 μM) while N-term-7 assayed by ATG4B cleavage assays showed improved inhibition for about 30% at 10 μM (FIGS. 22A and 22B). It was found that the inhibition of N-term-7 and N-term-9 were maintained even at a lower peptide concentration (1 μM) (FIG. 22B), which indicates the successful improvement of the designed peptide. These results suggested that the intermolecular allosteric regulation resulted from the binding of LC3B on the ATG4B N-terminal tail can exist and contribute to the enzyme activity of ATG4B, and the contribution can be precluded when the binding between LC3B and the ATG4B N-terminal tail was interfered by the peptides that bound to the LIR motif binding pocket on LC3B, which was confirmed by the NMR chemical shift perturbation assays described in the next section. FIGS. 22C and 22D show the three-dimensional structure information when N-term 7 and N-term 9 targeting LIR binding pocket on LC3B, respectively.

### Example 10: The Inhibitory Effect of the Designed Peptides to ATG4B Activity Resulted from their Binding to the LIR Binding Pocket in LC3B

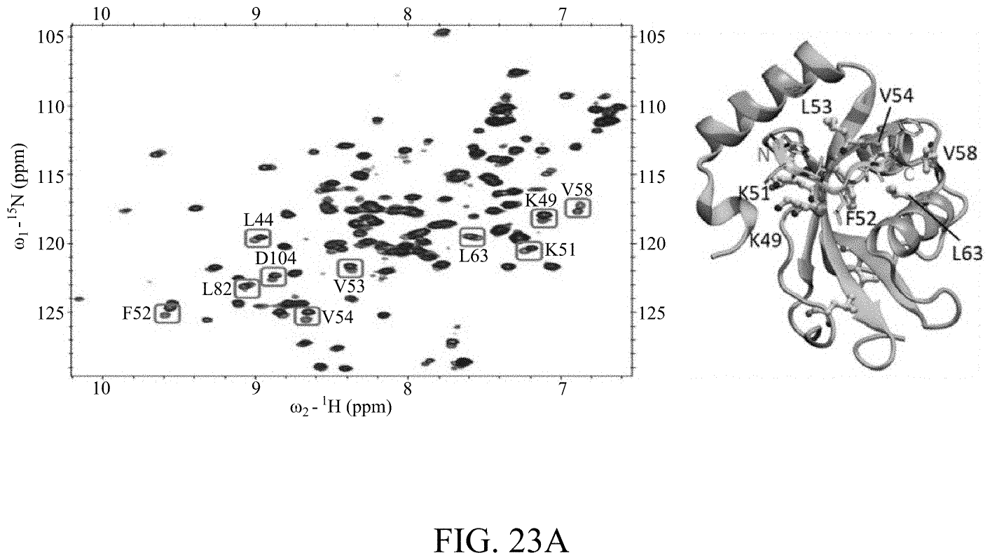

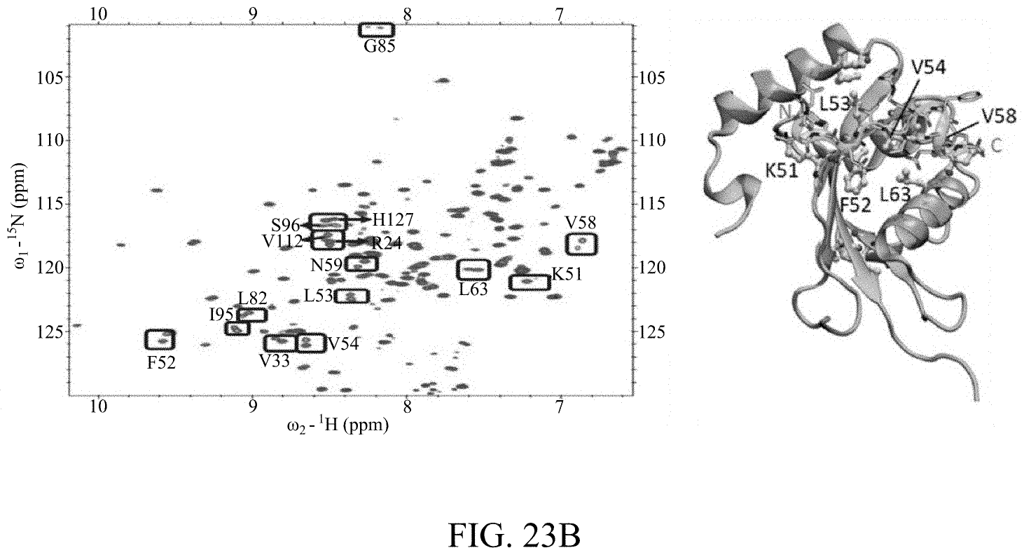

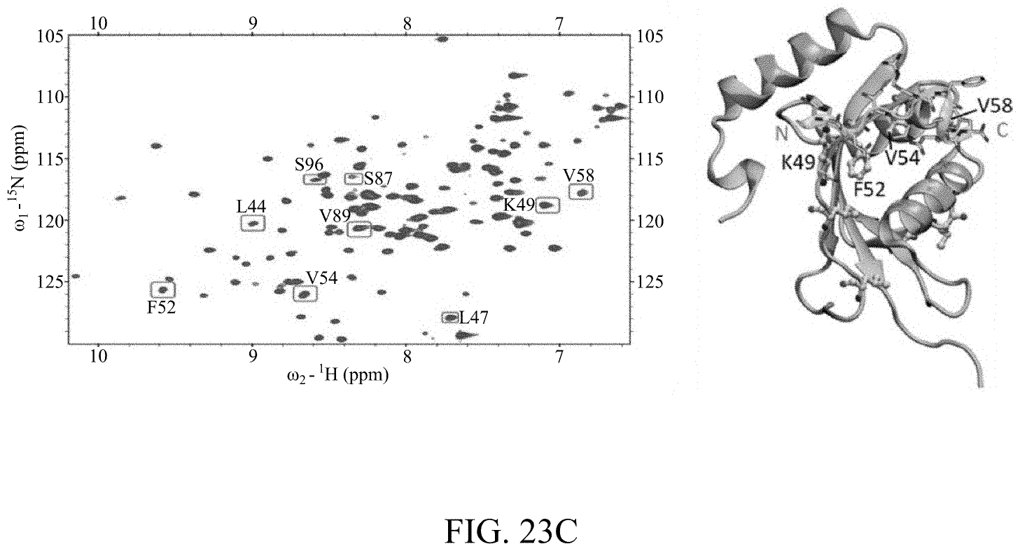

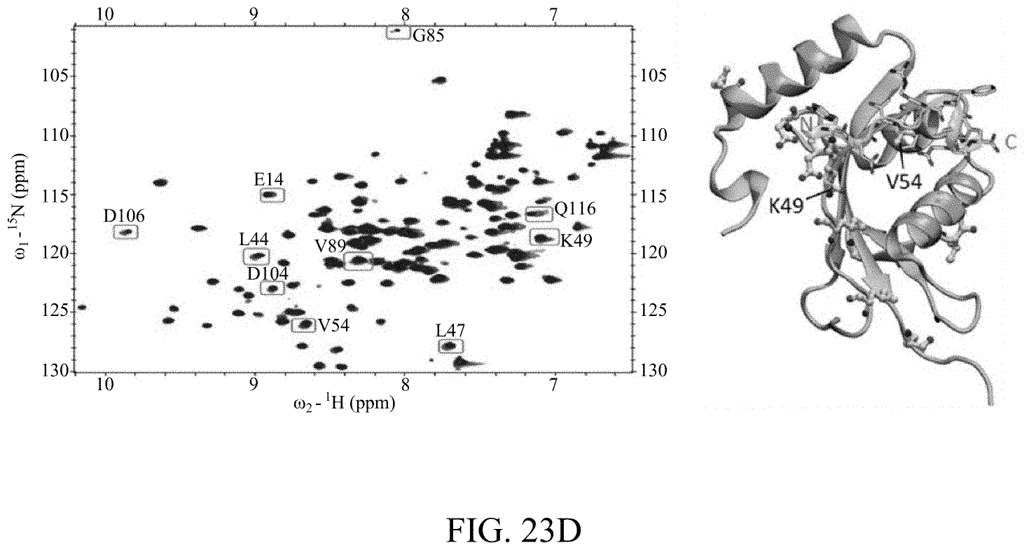

Since the peptides were designed to target the LIR motif binding pocket on LC3B, their ATG4B inhibition ability was presumed to come from the interference of the interaction between the ATG4B N-terminal tail and LC3B, which shifted the ATG4B back to its closed/inactive conformation through the intermolecular allosteric regulation accompanied by the folded-back N-terminal tail. To confirm the molecular mechanism behind the observed reduction of ATG4B activity by the peptides, NMR spectra were analyzed when ¹⁵N-labelled LC3B was titrated with the peptides to identify their binding sites on LC3B. It was observed that N-term-18 introduced a similar pattern of chemical shift perturbation at the LIR motif binding pocket (FIG. 23B). When titrated with N-term-7, the residues with large chemical shift perturbations were located at the same binding pocket and consistent with the computational prediction of the preferred binding site for N-term-7 (FIG. 23A). By mapping the perturbed residues on the LC3B from the last snapshot of 100 ns LC3B-N-term-7 complex simulation, the perturbed sites were located on the region that formed stable interaction with N-term-7 (FIG. 23A). Similar perturbations were shared in TAT-introduced (to increase the cell penetration, see next section) N-term-9 (TAT-N-term-9) (FIG. 23C) but less overlapped with the perturbed residues by TAT-only peptide (TAT-Ctrl) (FIG. 23D). These results suggested that the designed peptides can bind and compete with the ATG4B N-terminal tail for the LIR motif binding pocket on LC3B, which contributed to the observed reduction of ATG4B activity upon treatment of peptides.

### Example 11: The Designed Peptides Inhibited the Viability of Cancer Cell Line

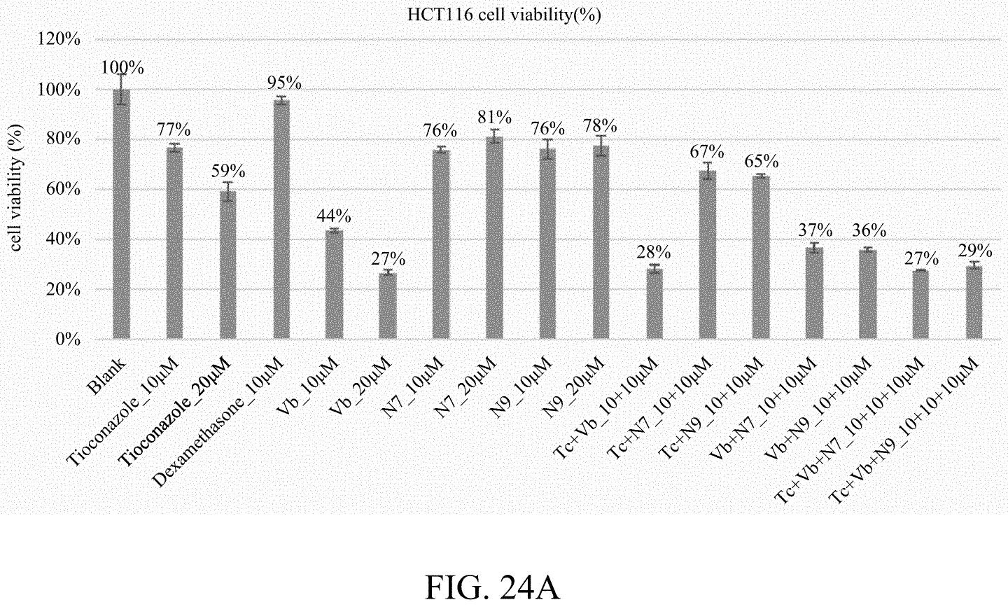

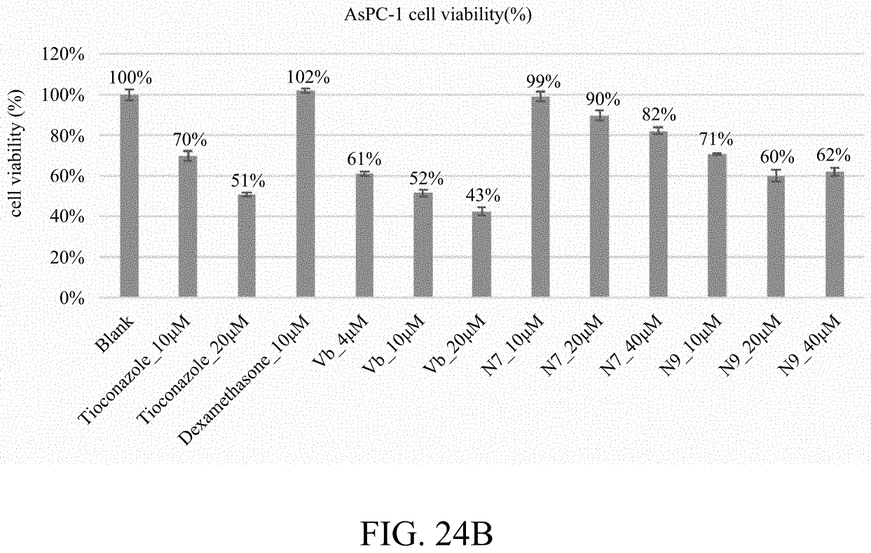

The existence of the intermolecular allosteric mechanism of ATG4B induced by LC3B supported by the results revealed a new strategy to design ATG4B inhibitors for cancer therapy. An allosteric drug that is designed for interfering the binding of the ATG4B N-terminal tail by LC3B might affect the tumor growth due to the impairment of the ATG4B enzyme activity. As a proof of the concept, the effect of the designed peptides on the suppression of the cancer cells was tested by the cell viability assays. Different concentrations of N-term-7 and N-term-9 were treated to the colorectal cancer, HCT116, and the pancreatic cancer, AsPC-1, cell lines (FIGS. 24A and 24B). In an embodiment of the present disclosure, increasing the cell penetration of the peptides was considered. To achieve this, a “TAT” sequence (SEQ ID NO: 23 (YGRKKRRQRRR)) followed by a “GGS” linker was introduced in front of the peptide sequences (e.g., TAT-N-term-7, with an amino acid sequence of SEQ ID NO: 16 (YGRKKRRQRRR-GGS-YDTLGIF) and TAT-N-term-9, with an amino acid sequence of SEQ ID NO: 17 (YGRKKRRQRRR-GGS-YDTLRFAEF)). The introduced TAT sequence did not impair the binding of the peptide on LC3B, which was confirmed by the NMR chemical shift perturbation (FIG. 23C). With TAT, both peptides showed moderate inhibition on the cell viability of HCT116 by 24% at the concentration of 10 μM. The inhibition was not contributed by the introduced “TAT” cell penetrating peptide and the “GGS” linker (TAT-Ctrl) (FIGS. 24A and 24B). When co-treated with tioconazole (Tc), the drug that served as the orthosteric inhibitor of ATG4B, the peptides showed an additive effect on the cell viability of HCT116 by a decrease of 35% for TAT-N-term-9 and 33% for TAT-N-term-7 when using 10 μM peptides with 10 μM Tc, compared to that of 33% decrease for 10 μM Tc only (FIG. 24A). The suppression of the cell viability upon treatments of peptides was also observed in AsPC-1 cancer cells, where TAT-N-term-7 required a higher concentration to show apparent inhibition (by 18% at 40 μM) while TAT-N-term-9 gave a better inhibition than N-term-7 even at a lower concentration (by around 30% at 10 μM) (FIG. 24B). These results suggested that the peptides designed for interfering the LC3B-mediated allosteric regulation of ATG4B could suppress the viability of the cancer cells, which implied that a drug-like inhibitor for ATG4B functioning through a similar mechanism as an allosteric modulator might be applied to the cancer therapy.

### Example 12: Prediction of the Allosteric Site(s) in ATG4B by Time-Dependent Linear Response Theory (Td-LRT)

An intramolecular allosteric site of a disease target protein can be predicted and validated via the time-dependent linear response theory (td-LRT)-based method. Details of the method are described by Huang et al. (Huang et al., 2019, bioRxiv). In this Example, the clustered intramolecular communication centers (ICCs) as potential allosteric sites were predicted in a notable distance away from the catalytic center, so as to find the drugs that can bind the sites. A library of 2016 FDA-approved drugs by small molecule docking was screened for this purpose. These drugs were then tested for their in vitro inhibition of ATG4B's function by enzymatic assay and suppression of tumor cell growth.

By perturbing 41 residues within 8 Å from Cys74, the catalytic cysteine of ATG4B, the td-LRT-based method was used to derive the coarse-grained communication map (CGCM) (FIG. 25A) where a group of “hot-spots” near the residue 30 in the off-diagonals of CGCM can be found. Among the top 10 ICCs, 6 were spatially close enough to be clustered into a single site as shown in FIGS. 25A and 25B. The docking study (see MATERIALS AND METHODS) found, from 2016 FDA drugs, only 6 FDA drugs having relatively high affinity (<-7 kcal/mol) and site proximity (<7 Å) that could bind the site (Table 16). In the 6 FDA drugs, fenretinide, moxidectin and conivaptan were among the strongest three to bind the allosteric site in ATG4B's active (open) form (PDB ID: 2Z0D), considering both the binding affinity and their pose-site distances (Table 16; also see MATERIALS AND METHODS for definition). The closed form of ATG4B (PDB ID: 2CY7) was also used to conduct the same screening procedure. The results showed that fenretinide, moxidectin and conivaptan can still be found that have relatively high affinity (<−7 kcal/mol) and site proximity (<7 Å) against the closed form, but not the other three drugs (Table 16).

TABLE 16
Top 6 FDA-approved drugs that bind ATG4B's
open form via the allosteric site

| | | Binding | Drug-Site | |
|---|---|---|---|---|
| | Drug | Affinity | Distance^{#} | Ranking |
| Rank | Name | (kcal/mol) | (Å) | Score |
| 1 | Fenretinide | −8.2 | 5.0 (6.1) | 1.86 |
| 2 | Moxidectin | −8.0 | 6.3 (6.0) | 1.43 |
| 3 | Conivaptan | −9.0 | 6.7 (7.6) | 1.14 |
| 4 | Lusutrombopag | −7.4 | 6.4 | 0.86 |
| 5 | Halcinonide | −7.3 | 6.3 | 0.86 |
| 6 | Butaclamol | −7.3 | 6.5 | 0.57 |

^{#}The distance between the mass center of a drug docking pose to its closest heavy atom of allosteric site residues. The distances in the parentheses are the same after a 10-ns MD simulation.

Furthermore, MD simulations for these three drugs were conducted to examine their binding stability and site proximity. The top two results based on the heavy-atom contact and proximity to the allosteric site (FIG. 25C) were taken for further enzymatic assays and tumor suppression tests. In these two, moxidectin has a marginally higher contact (41 vs. 38) and closer site proximity (6.0 Å vs. 6.1 Å) than fenretinide. It is also found to inhibit ATG4B catalysis by 36% and >80% at 10 and 20 μM, respectively (FIG. 25E), while suppress tumor growth better in the pancreatic and colorectal cancer cell lines than fenretinide that seems to be a better breast cancer inhibitor (FIG. 25F). When 4 μM Mx is co-used with 500 nM tioconazole, it shows an improved tumor suppression efficacy as compared with individual treatment alone (FIG. 25G).

### Example 13: The Interface Blocker of 3CL^{pro}

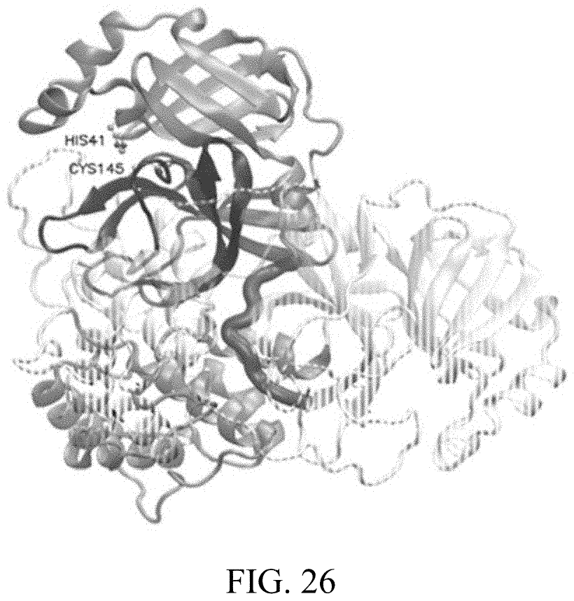

FIG. 26 illustrated a homodimer structure of 3CL^{pro}, where the three domains are labeled by different colors. The dimer interface was located between the domain II of one monomer and N-terminus in another monomer. The MD simulations provided a more physiologically realistic environment than the environment (in vacuum), wherein small-molecule docking was conducted. The mean drug-interface contacts derived from MD simulations could provide a guideline for drug screening. Based on the global docking and drug contact analysis, drug-interface contacts for the domain II and for the N-terminus were calculated, where a drug having a higher drug-interface contact could suggest higher interaction-interference caused by the drug. Table 17 showed examples of top-10 drugs ranked by drug-interface contact for both interfaces.

TABLE 17
The top-10 drugs ranked by drug-interface contact for both interfaces

| | Drug | Drug | Domain II | | Drug | Drug | N-terminus |
|---|---|---|---|---|---|---|---|
| | ID | Name | Contact | | ID | Name | Contact |
| 1 | 02006 | Icatibant | 37 | 1 | 02009 | Norvancomycin | 34 |
| 2 | 02009 | Norvancomycin | 36 | 2 | 02011 | Fondaparinux | 31 |
| 3 | 01684 | Kanamycin | 35 | 3 | 02006 | Icatibant | 31 |
| 4 | 00849 | Warfarin | 35 | 4 | 01973 | Ombitasvir | 29 |
| 5 | 00452 | Pidotimod | 35 | 5 | 01982 | Carbetocin | 28 |
| 6 | 00622 | Tolcapone | 35 | 6 | 01067 | Raltitrexed | 28 |
| 7 | 00042 | Pyrazinamide | 35 | 7 | 01916 | Ritonavir | 28 |
| 8 | 02012 | Daptomycin | 33 | 8 | 01693 | Dasatinib | 27 |
| 9 | 00060 | Allopurinol | 33 | 9 | 01574 | Bictegravir | 27 |
| 10 | 00212 | Dacarbazine | 32 | 10 | 01951 | Glycyrrhizic | 26 |
| | | | | | | acid | |

Further, not only top-10 drugs but also top-50 drugs ranked by drug-interface contacts were selected to perform the MD simulation and MM/PB(GB)SA analysis using the standard protocol. From the simulation trajectory, the mean drug-interface contacts were calculated by the average drug-interface contacts over the snapshots of the last 2 ns of MD between the 3CL^{pro} interface and drugs. Top-10 drugs ranked by mean drug-interface contacts were selected for each interface as shown in Tables 18 and 19 below.

Tables 18 and 19 below showed the mean drug-interface contacts between drug and domain-II/N-terminus, and corresponding MD-based energy. The “Contact” referred to the mean drug-interface contacts calculated over the snapshots of the last 2 ns of MD trajectories. The column MMG(P)BSA was the MMG(P)BSA-derived energy in the unit of kcal/mol. It was observed that the top-10 drugs ranked by drug-interface contacts shown in Table 17 may not be top-10 when the ranking was based on the mean drug-interface contacts, and generally, drugs with high mean drug-interface contacts had low MD/MMP(G)BSA derived energy, namely good drug-protein affinity.

TABLE 18
The mean drug-interface contacts between drug and
domain-II, and corresponding MD based energy

| | Drug | Drug | | | |
|---|---|---|---|---|---|
| | ID | Name | Contact | MMGBSA | MMPBSA |
| 1 | 01995 | Anidulafungin | 27.80 | −29.71 ± 2.79 | −17.47 ± 5.50 |
| 2 | 00297 | Miglitol | 22.25 | −21.99 ± 3.24 | −19.50 ± 3.05 |
| 3 | 01959 | Spiramycin I | 18.80 | −36.45 ± 5.02 | −19.87 ± 5.54 |
| 4 | 00755 | Alosetron | 18.25 | −33.60 ± 3.52 | −26.32 ± 3.48 |
| 5 | 01987 | Felypressin | 17.55 | −30.34 ± 5.20 | −22.14 ± 4.98 |
| 6 | 00152 | 6-thioguanine | 14.95 | −20.41 ± 3.31 | −14.70 ± 3.45 |
| 7 | 01719 | Taurohyodeoxycholic | 14.30 | −44.99 ± 3.80 | −28.29 ± 4.75 |
| | | acid | | | |
| 8 | 01988 | Ornipressin | 13.40 | −37.88 ± 4.28 | −31.47 ± 4.48 |
| 9 | 01858 | Gamma-oryzanol | 13.00 | −25.25 ± 2.95 | −22.65 ± 2.31 |
| 10 | 00489 | Lacosamide | 12.70 | −22.72 ± 2.26 | −16.44 ± 2.68 |

TABLE 19
The mean drug-interface contacts between drug and
N-terminus, and corresponding MD based energy

| | Drug | Drug | | | |
|---|---|---|---|---|---|
| | ID | Name | Contact | MMGBSA | MMPBSA |
| 1 | 01973 | Ombitasvir | 31.90 | −30.02 ± 2.62 | −22.03 ± 2.66 |
| 2 | 02009 | Norvancomycin | 29.65 | −26.07 ± 3.56 | −18.28 ± 2.54 |
| 3 | 01324 | Lubiprostone | 24.35 | −24.89 ± 2.76 | −18.46 ± 2.80 |
| 4 | 01988 | Ornipressin | 23.80 | −55.73 ± 4.06 | −33.04 ± 5.57 |
| 5 | 02006 | Icatibant | 20.45 | −46.10 ± 5.60 | −29.86 ± 5.47 |
| 6 | 00755 | Alosetron | 20.30 | −29.40 ± 1.57 | −23.98 ± 2.00 |
| 7 | 01991 | Caspofungin | 18.35 | −54.00 ± 3.82 | −37.49 ± 4.48 |
| 8 | 01990 | Plicamycin | 18.20 | −41.53 ± 3.66 | −21.20 ± 3.58 |
| 9 | 01604 | Minocycline | 17.65 | −28.90 ± 3.55 | −16.70 ± 4.87 |
| 10 | 00438 | Telbivudine | 17.40 | −29.28 ± 2.20 | −22.58 ± 2.62 |

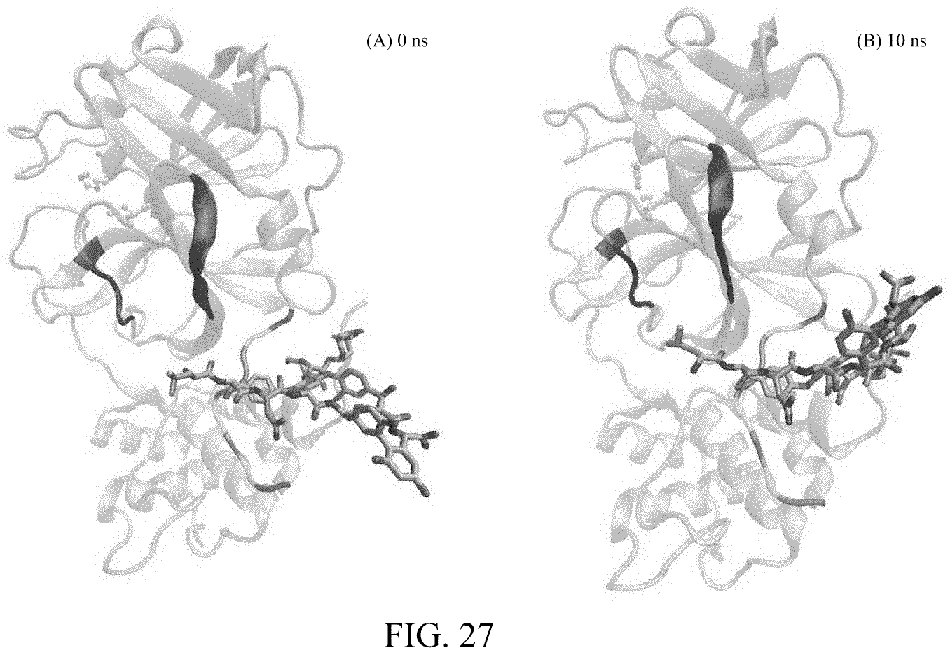

From the above results, the drug norvancomycin (02009) revealed high contacts with the N-terminus (orange color in FIGS. 27(A) and 27(B)) in both global docking (rank 1 of Table 17) and MD simulations (rank 2 of Table 19). Further, the MD snapshots shown in FIGS. 27(A) and 27(B) demonstrated that norvancomycin tightly bind with the N-terminus interface residues in physiologically realistic environment.

### Example 14: Verification of the Interface Blocker Using In Vitro 3C-Like Protease Activity Assay

From the top-2 that had the most contact with 3CL^{pro}'s domain-II and N-terminus, four interface drugs, namely anidulafungin (01995), miglitol (00297), ombitasvir (01973) and norvancomycin (02009) were obtained, where norvancomycin was the first drug chosen for the following enzyme activity assay.

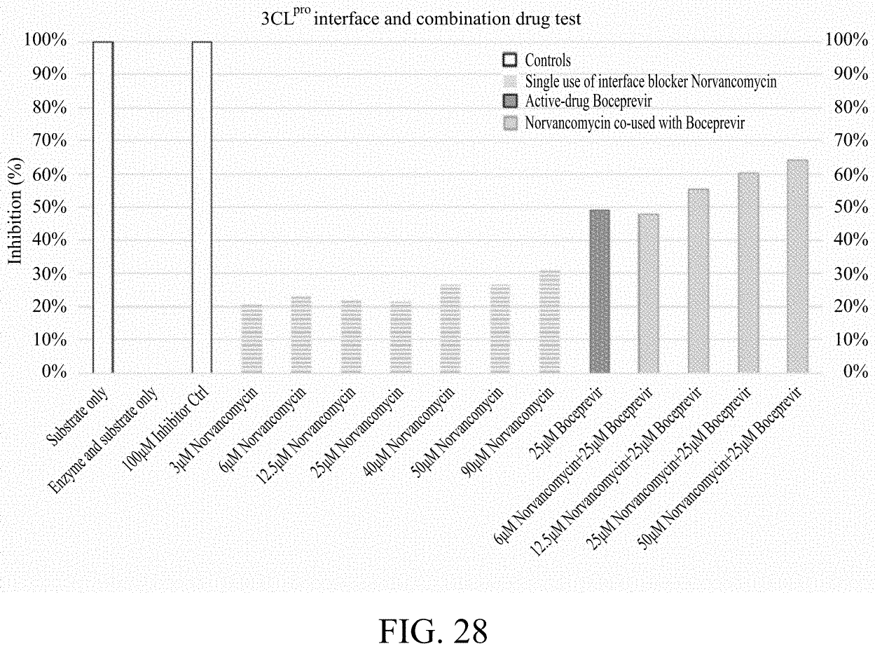

A 3CL^{pro} (Cat. #78042-1) assay kit (BPS Bioscience, CA, USA) was used for the single use experiment of the norvancomycin, and it showed 20 to 30% inhibition at 3 to 90 μM (see the yellow bars in the middle of FIG. 28). For validating the combination effect on an interface blocker, 25 μM boceprevir, a published drug targeted to 3CL^{pro} active site, was combined with norvancomycin at different concentrations from 6 μM to 50 μM. The result showed that the predicted interface blocker co-used with the active-site inhibitor at the molar ratio of 1:1 elevated the inhibition by around 10%, while 50 μM norvancomycin gave another 17% inhibition in combination with 25 μM boceprevir (FIG. 28).

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as” and “for example”) provided herein, is intended merely to better illuminate the disclosure and does not pose a limitation on the scope of the disclosure unless otherwise indicated.

What is claimed is:

1. A method for determining a drug combination targeting different sites of a protein or a protein complex, comprising:
identifying a first binding site and a second binding site of the protein or the protein complex based on a three-dimensional structure thereof, wherein the first binding site and the second binding site are the different sites in the three-dimensional structure of the protein or the protein complex;
identifying a first drug interacting with the first binding site;
identifying a second drug interacting with the second binding site; and
combining the first drug and the second drug to provide at least one of a synergistic effect and an additive effect in suppressing an activity of the protein or the protein complex.

2. The method according to claim 1, wherein the first binding site and the second binding site are independently a main functional site, an orthosteric site, an active site, a main substrate-binding site, an allosteric site, a recognition site or a site at a protein-protein interface.

3. The method according to claim 1, wherein the first drug and the second drug are independently selected from the group consisting of a newly synthesized compound, an FDA-approved drug, an FDA-approved biologic, a drug metabolite, a prodrug, an experimental small molecule, an experimental biologic, an experimental polypeptide, and any combination thereof.

4. The method according to claim 1, wherein at least one of the identifying of the first drug and the identifying of the second drug comprises selecting at least one of the first drug and the second drug from at least one dataset.

5. The method according to claim 4, wherein the at least one dataset is a drug library, a genomic dataset, a proteomic dataset, a biochemical dataset, or a population dataset.

6. The method according to claim 4, wherein the selecting comprises interacting molecular entities of the dataset with at least one of the first binding site and the second binding site, and ranking the affinity of the molecular entities to at least one of the first binding site and the second binding site by an experimental and/or theoretical method.

7. The method according to claim 6, wherein the experimental and/or theoretical method is one selected from the group consisting of nuclear magnetic resonance (NMR) spectroscopy, isothermal titration calorimetry, docking energy, distances between poses and the binding site, entropy calculations, molecular dynamics (MD) simulations, normal mode analysis (NMA), and any combination thereof.

8. The method according to claim 7, wherein the allosteric site is determined by analysis of atomic displacement and/or correlated atomic motion derived from the molecular dynamics (MD) simulations, normal mode analysis, linear response theory (LRT), or any combination thereof.

9. The method according to claim 6, wherein the poses are reported for the molecular entities docked by the AutoDock Vina and/or AutoDock.

10. The method according to claim 6, wherein the ranking is performed by normalized ranking, logarithm of odds (LOD) scoring, or a combination thereof.

11. The method according to claim 10, wherein the normalized ranking is performed based on at least one of docking affinity, number of contacts, and an extent of poses concentrated in at least one of the first binding site and the second binding site.

12. The method according to claim 10, wherein the logarithm of odds scoring is performed based on at least one of docking affinity, a distance of the molecular entity to the first binding site or the second binding site, and a size of poses cluster.

13. A method for treating an autophagy related 4B cysteine peptidase (ATG4B)-related disease or a 3CL protease (3CL^{pro})-related disease in a subject in need thereof, comprising administering an effective amount of the drug combination obtained from the method of claim 1.

14. The method according to claim 13, wherein the drug combination comprises at least two selected from the group consisting of aclacinomycin A, boceprevir, daclatasvir, dihydroergocristine, ethynyl estradiol, Evans blue, moxidectin, netupitant, norvancomycin, ponatinib, temsirolimus, tioconazole, vinorelbine, tat-N7 peptide, and tat-N9 peptide.

15. The method according to claim 13, wherein the ATG4B-related disease is breast cancer, colorectal cancer, neural glioma cancer, gastric cancer, pancreatic cancer, or melanoma.


SEQUENCE LISTING (23 sequences)

SEQ ID NO: 1 - protein, 7 aa, synthetic construct
YDYLGGF

SEQ ID NO: 2 - protein, 7 aa, synthetic construct
YDTLGIF

SEQ ID NO: 3 - protein, 7 aa, synthetic construct
YDTLYGF

SEQ ID NO: 4 - protein, 7 aa, synthetic construct
YDTLSGF

SEQ ID NO: 5 - protein, 7 aa, synthetic construct
YPTLGGF

SEQ ID NO: 6 - protein, 7 aa, synthetic construct
PDTLGGF

SEQ ID NO: 7 - protein, 7 aa, synthetic construct
YDTLGGP

SEQ ID NO: 8 - protein, 7 aa, synthetic construct
YDTLTGF

SEQ ID NO: 9 - protein, 7 aa, synthetic construct
YDTPGGF

SEQ ID NO: 10 - protein, 7 aa, synthetic construct
YDTSGGF

SEQ ID NO: 11 - protein, 7 aa, synthetic construct
YDWLGGF

SEQ ID NO: 12 - protein, 7 aa, synthetic construct
YWTLGGF

SEQ ID NO: 13 - protein, 7 aa, synthetic construct
YYTLGGF

SEQ ID NO: 14 - protein, 7 aa, synthetic construct
YDTLGGF

SEQ ID NO: 15 - protein, 14 aa, Betacoronavirus
KTSAVLQSGFRKME

SEQ ID NO: 16 - protein, 21 aa, synthetic construct
YGRKKRRQRRRGGSYDTLGIF

SEQ ID NO: 17 - protein, 23 aa, synthetic construct
YGRKKRRQRRRGGSYDTLRFAEF

SEQ ID NO: 18 - protein, 12 aa, synthetic construct
TLTYDTLRFAEF

SEQ ID NO: 19 - protein, 18 aa, synthetic construct
MDAATLTYDTLRFAEFED

SEQ ID NO: 20 - protein, 9 aa, synthetic construct
YDTLRFAEF

SEQ ID NO: 21 - protein, 6 aa, synthetic construct
YDTLGF

SEQ ID NO: 22 - protein, 8 aa, synthetic construct
YDTLGGGF

SEQ ID NO: 23 - protein, 11 aa, synthetic construct
YGRKKRRQRRR